SEQUENCE LISTING (79 sequences)

SEQ ID NO: 1 - protein, 394 aa, Homo sapiens
MFCPLKLILLPVLLDYSLGLNDLNVSPPELTVHVGDSALMGCVFQSTEDKCIFKIDWTLSPGEHAKDEYVLYYYSNLSVPIGRFQNRVHLMGDILCNDGSLLLQDVQEADQGTYICEIRLKGESQVFKKAVVLHVLPEEPKELMVHVGGLIQMGCVFQSTEVKHVTKVEWIFSGRRAKEEIVFRYYHKLRMSVEYSQSWGHFQNRVNLVGDIFRNDGSIMLQGVRESDGGNYTCSIHLGNLVFKKTIVLHVSPEEPRTLVTPAALRPLVLGGNQLVIIVGIVCATILLLPVLILIVKKTCGNKSSVNSTVLVKNTKKTNPEIKEKPCHFERCEGEKHIYSPIIVREVIEEEEPSEKSEATYMTMHPVWPSLRSDRNNSLEKKSGGGMPKTQQAF

SEQ ID NO: 2 - protein, 344 aa, Homo sapiens
MALLLCFVLLCGVVDFARSLSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAEMTSSVISVKNASSEYSGTYSCTVRNRVGSDQCLLRLNVVPPSNKAGLIAGAIIGTLLALALIGLIIFCCRKKRREEKYEKEVHHDIREDVPPPKSRTSTARSYIGSNHSSLGSMSPSNMEGYSKTQYNQVPSEDFERTPQSPTLPPAKFKYAY

SEQ ID NO: 3 - protein, 373 aa, Homo sapiens
MSLLLLLLLVSYYVGTLGTHTEIKRVAEEKVTLPCHHQLGLPEKDTLDIEWLLTDNEGNQKVVITYSSRHVYNNLTEEQKGRVAFASNFLAGDASLQIEPLKPSDEGRYTCKVKNSGRYVWSHVILKVLVRPSKPKCELEGELTEGSDLTLQCESSSGTEPIVYYWQRIREKEGEDERLPPKSRIDYNHPGRVLLQNLTMSYSGLYQCTAGNEAGKESCVVRVTVQYVQSIGMVAGAVTGIVAGALLIFLLVWLLIRRKDKERYEEEERPNEIREDAEAPKARLVKPSSSSSGSRSSRSGSSSTRSTANSASRSQRTLSTDAAPQPGLATQAYSLVGPEVRGSEPKKVHHANLTKAETTPSMIPSQSRAFQTV

SEQ ID NO: 4 - protein, 390 aa, Homo sapiens
MISLPGPLVTNLLRFLFLGLSALAPPSRAQLQLHLPANRLQAVEGGEVVLPAWYTLHGEVSSSQPWEVPFVMWFFKQKEKEDQVLSYINGVTTSKPGVSLVYSMPSRNLSLRLEGLQEKDSGPYSCSVNVQDKQGKSRGHSIKTLELNVLVPPAPPSCRLQGVPHVGANVTLSCQSPRSKPAVQYQWDRQLPSFQTFFAPALDVIRGSLSLTNLSSSMAGVYVCKAHNEVGTAQCNVTLEVSTGPGAAVVAGAVVGTLVGLGLLAGLVLLYHRRGKALEEPANDIKEDAIAPRTLPWPKSSDTISKNGTLSSVTSARALRPPHGPPRPGALTPTPSLSSQALPSPRLPTTDGAHPQPISPIPGGVSSSGLSRMGAVPVMVPAQSQAGSLV

SEQ ID NO: 5 - protein, 319 aa, Homo sapiens
MVGKMWPVLWTLCAVRVTVDAISVETPQDVLRASQGKSVTLPCTYHTSTSSREGLIQWDKLLLTHTERVVIWPFSNKNYIHGELYKNRVSISNNAEQSDASITIDQLTMADNGTYECSVSLMSDLEGNTKSRVRLLVLVPPSKPECGIEGETIIGNNIQLTCQSKEGSPTPQYSWKRYNILNQEQPLAQPASGQPVSLKNISTDTSGYYICTSSNEEGTQFCNITVAVRSPSMNVALYVGIAVGVVAALIIIGIIIYCCCCRGKDDNTEDKEDARPNREAYEEPPEQLRELSREREEEDDYRQEEQRSTGRESPDHLDQ

SEQ ID NO: 6 - protein, 299 aa, Homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGVIVAAVLVTLILLGILVFGIWFAYSRGHFDRTKKGTSSKKVIYSQPSARSEGEFKQTSSFLV

SEQ ID NO: 7 - protein, 298 aa, Homo sapiens
MARRSRHRLLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLNISGIIAAVVVVALVISVCGLGVCYAQRKGYFSKETSFQKSNSSSKATTMSENDFKHTKSFII

SEQ ID NO: 8 - protein, 310 aa, Homo sapiens
MALRRPPRLRLCARLPDFFLLLLFRGCLIGAVNLKSSNRTPVVQEFESVELSCIITDSQTSDPRIEWKKIQDEQTTYVFFDNKIQGDLAGRAEILGKTSLKIWNVTRRDSALYRCEVVARNDRKEIDEIVIELTVQVKPVTPVCRVPKAVPVGKMATLHCQESEGHPRPHYSWYRNDVPLPTDSRANPRFRNSSFHLNSETGTLVFTAVHKDDSGQYYCIASNDAGSARCEEQEMEVYDLNIGGIIGGVLVVLAVLALITLGICCAYRRGYFINNKQDGESYKNPGKPDGVNYIRTDEEGDFRHKSSFVI

SEQ ID NO: 9 - protein, 423 aa, Homo sapiens
MVFAFWKVFLILSCLAGQVSVVQVTIPDGFVNVTVGSNVTLICIYTTTVASREQLSIQWSFFHKKEMEPISHSSCLSTEGMEEKAVSQCLKMTHARDARGRCSWTSEIYFSQGGQAVAIGQFKDRITGSNDPGNASITISHMQPADSGIYICDVNNPPDFLGQNQGILNVSVLVKPSKPLCSVQGRPETGHTISLSCLSALGTPSPVYYWHKLEGRDIVPVKENFNPTTGILVIGNLTNFEQGYYQCTAINRLGNSSCEIDLTSSHPEVGIIVGALIGSLVGAAIIISVVCFARNKAKAKAKERNSKTIAELEPMTKINPRGESEAMPREDATQLEVTLPSSIHETGPDTIQEPDYEPKPTQEPAPEPAPGSEPMAVPDLDIELELEPETQSELEPEPEPEPESEPGVVVEPLSEDEKGVVKA

SEQ ID NO: 10 - protein, 327 aa, Homo sapiens
MAELPGPFLCGALLGFLCLSGLAVEVKVPTEPLSTPLGKTAELTCTYSTSVGDSFALEWSFVQPGKPISESHPILYFTNGHLYPTGSKSKRVSLLQNPPTVGVATLKLTDVHPSDTGTYLCQVNNPPDFYTNGLGLINLTVLVPPSNPLCSQSGQTSVGGSTALRCSSSEGAPKPVYNWVRLGTFPTPSPGSMVQDEVSGQLILTNLSLTSSGTYRCVATNQMGSASCELTLSVTEPSQGRVAGALIGVLLGVLLLSVAAFCLVRFQKERGKKPKETYGGSDLREDAIAPGISEHTCMRADSSKGFLERPSSASTVTTTKSKLPMVV

SEQ ID NO: 11 - protein, 430 aa, Homo sapiens
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 12 - protein, 399 aa, Homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 13 - protein, 414 aa, Homo sapiens
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 14 - protein, 442 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDHAVIGGVVAVVVFAMLCLLIILGRYFARHKGTYFTHEAKGADDAADADTAIINAEGGQNNSEEKKEYFI

SEQ ID NO: 15 - protein, 435 aa, Homo sapiens
MIWKRSAVLRFYSVCGLLLQGSQGQFPLTQNVTVVEGGTAILTCRVDQNDNTSLQWSNPAQQTLYFDDKKALRDNRIELVRASWHELSISVSDVSLSDEGQYTCSLFTMPVKTSKAYLTVLGVPEKPQISGFSSPVMEGDLMQLTCKTSGSKPAADIRWFKNDKEIKDVKYLKEEDANRKTFTVSSTLDFRVDRSDDGVAVICRVDHESLNATPQVAMQVLEIHYTPSVKIIPSTPFPQEGQPLILTCESKGKPLPEPVLWTKDGGELPDPDRMVVSGRELNILFLNKTDNGTYRCEATNTIGQSSAEYVLIVHDVPNTLLPTTIIPSLTTATVTTTVAITTSPTTSATTSSIRDPNALAGQNGPDHALIGGIVAVVVFVTLCSIFLLGRYLARHKGTYLTNEAKGAEDAPDADTAIINAEGSQVNAEEKKEYFI

SEQ ID NO: 16 - protein, 432 aa, Homo sapiens
MGAPAASLLLLLLLFACCWAPGGANLSQDGYWQEQDLELGTLAPLDEAISSTVWSSPDMLASQDSQPWTSDETVVAGGTVVLKCQVKDHEDSSLQWSNPAQQTLYFGEKRALRDNRIQLVTSTPHELSISISNVALADEGEYTCSIFTMPVRTAKSLVTVLGIPQKPIITGYKSSLREKDTATLNCQSSGSKPAARLTWRKGDQELHGEPTRIQEDPNGKTFTVSSSVTFQVTREDDGASIVCSVNHESLKGADRSTSQRIEVLYTPTAMIRPDPPHPREGQKLLLHCEGRGNPVPQQYLWEKEGSVPPLKMTQESALIFPFLNKSDSGTYGCTATSNMGSYKAYYTLNVNDPSPVPSSSSTYHAIIGGIVAFIVFLLLIMLIFLGHYLIRHKGTYLTHEAKGSDDAPDADTAIINAEGGQSGGDDKKEYFI

SEQ ID NO: 17 - protein, 388 aa, Homo sapiens
MGRARRFQWPLLLLWAAAAGPGAGQEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAATLRWYRDRKELKGVSSSQENGKVWSVASTVRFRVDRKDDGGIIICEAQNQALPSGHSKQTQYVLDVQYSPTARIHASQAVVREGDTLVLTCAVTGNPRPNQIRWNRGNESLPERAEAVGETLTLPGLVSADNGTYTCEASNKHGHARALYVLVVYDPGAVVEAQTSVPYAIVGGILALLVFLIICVLVGMVWCSVRQKGSYLTHEASGLDEQGEAREAFLNGSDGHKRKEEFFI

SEQ ID NO: 18 - protein, 393 aa, Homo sapiens
MWWRVLSLLAWFPLQEASLTNHTETITVEEGQTLTLKCVTSLRKNSSLQWLTPSGFTIFLNEYPALKNSKYQLLHHSANQLSITVPNVTLQDEGVYKCLHYSDSVSTKEVKVIVLATPFKPILEASVIRKQNGEEHVVLMCSTMRSKPPPQITWLLGNSMEVSGGTLHEFETDGKKCNTTSTLIIHTYGKNSTVDCIIRHRGLQGRKLVAPFRFEDLVTDEETASDALERNSLSSQDPQQPTSTVSVTEDSSTSEIDKEEKEQTTQDPDLTTEANPQYLGLARKKSGILLLTLVSFLIFILFIIVQLFIMKLRKAHVIWKKENEVSEHTLESYRSRSNNEETSSEEKNGQSSHPMRCMNYITKLYSEAKTKRKENVQHSKLEEKHIQVPESIV

SEQ ID NO: 19 - protein, 262 aa, Homo sapiens
MAWKSSVIMQMGRFLLLVILFLPREMTSSVLTVNGKTENYILDTTPGSQASLICAVQNHTREEELLWYREEGRVDLKSGNKINSSSVCVSSISENDNGISFTCRLGRDQSVSVSVVLNVTFPPLLSGNDFQTVEEGSNVKLVCNVKANPQAQMMWYKNSSLLDLEKSRHQIQQTSESFQLSITKVEKPDNGTYSCIAKSSLKTESLDFHLIVKDKTVGVPIEPIIAACVVIFLTLCFGLIARRKKIMKLCMKDKDPHSETAL

SEQ ID NO: 20 - protein, 585 aa, Homo sapiens
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQYQKEIMERPPPFKPPPPPIKYTCIQEPNESDLPYHEMETL

SEQ ID NO: 21 - protein, 269 aa, Homo sapiens
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDREP

SEQ ID NO: 22 - protein, 348 aa, Homo sapiens
MLCPWRTANLGLLLILTIFLVAEAEGAAQPNNSLMLQTSKENHALASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELLPVPGAKKSAKLYIPYIILTIIILTIVGFIWLLKVNGCRKYKLNKTESTPVVEEDEMQPYASYTEKNNPLYDTTNKVKASEALQSEVDTDLHTL

SEQ ID NO: 23 - protein, 271 aa, Homo sapiens
MSAPRLLISIIIMVSASSSSCMGGKQMTQNYSTIFAEGNISQPVLMDINAVLCCPPIALRNLIIITWEIILRGQPSCTKAYKKETNETKETNCTVERITWVSRPDQNSDLQIRPVDTTHDGYYRGIVVTPDGNFHRGYHLQVLVTPEVNLFQSRNITAVCKAVTGKPAAQISWIPEGSILATKQEYWGNGTVTVKSTCPWEGHKSTVTCHVSHLTGNKSLSVKLNSGLRTSGSPALSLLIILYVKLSLFVVILVTTGFVFFQRINHVRKVL

SEQ ID NO: 24 - protein, 336 aa, Homo sapiens
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 25 - protein, 326 aa, Homo sapiens
MRTEAQVPALQPPEPGLEGAMGHRTLVLPWVLLTLCVTAGTPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLAGILGVSGVLLFGCVYLLHLLRRHKHRPAPRLQPSRTSPQAPRARAWAPSQASQAALHVPYATINTSCRPATLDTAHPHGGPSWWASLPTHAAHRPQGPAAWASTPIPARGSFVSVENGLYAQAGERPPHTGPGLTLFPDPRGPRAMEGPLGVR

SEQ ID NO: 26 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGMSRNAIIFLVLGILVFLILLGIGIYFYWSKCSREVLWHCHLCPSSTEHASASANGHVSYSAVSRENSSSQDPQTEGTR

SEQ ID NO: 27 - protein, 517 aa, Homo sapiens
MARMGLAGAAGRWWGLALGLTAFFLPGVHSQVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGTYICEATNPIGTRSGQVEVNITEFPYTPSPPEHGRRAGPVPTAIIGGVAGSILLVLIVVGGIVVALRRRRHTFKGDYSTKKHVYGNGYSKAGIPQHHPPMAQNLQYPDDSDDEKKAGPLGGSSYEEEEEEEEGGGGGERKVGGPHPKYDEDAKRPYFTVDEAEARQDGYGDRTLGYQYDPEQLDLAENMVSQNDGSFISKKEWYV

SEQ ID NO: 28 - protein, 538 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 29 - protein, 549 aa, Homo sapiens
MARTLRPSPLCPGGGKAQLSSASLLGAGLLLQPPTPPPLLLLLFPLLLFSRLCGALAGPIIVEPHVTAVWGKNVSLKCLIEVNETITQISWEKIHGKSSQTVAVHHPQYGFSVQGEYQGRVLFKNYSLNDATITLHNIGFSDSGKYICKAVTFPLGNAQSSTTVTVLVEPTVSLIKGPDSLIDGGNETVAAICIAATGKPVAHIDWEGDLGEMESTTTSFPNETATIISQYKLFPTRFARGRRITCVVKHPALEKDIRYSFILDIQYAPEVSVTGYDGNWFVGRKGVNLKCNADANPPPFKSVWSRLDGQWPDGLLASDNTLHFVHPLTFNYSGVYICKVTNSLGQRSDQKVIYISDPPTTTTLQPTIQWHPSTADIEDLATEPKKLPFPLSTLATIKDDTIATIIASVVGGALFIVLVSVLAGIFCYRRRRTFRGDYFAKNYIPPSDMQKESQIDVLQQDELDSYPDSVKKENKNPVNNLIRKDYLEEPEKTQWNNVENLNRFERPMDYYEDLKMGMKFVSDEHYDENEDDLVSHVDGSVISRREWYV

SEQ ID NO: 30 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 31 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 32 - protein, 366 aa, Homo sapiens
MALPPGPAALRHTLLLLPALLSSGWGELEPQIDGQTWAERALRENERHAFTCRVAGGPGTPRLAWYLDGQLQEASTSRLLSVGGEAFSGGTSTFTVTAHRAQHELNCSLQDPRSGRSANASVILNVQFKPEIAQVGAKYQEAQGPGLLVVLFALVRANPPANVTWIDQDGPVTVNTSDFLVLDAQNYPWLTNHTVQLQLRSLAHNLSVVATNDVGVTSASLPAPGLLATRVEVPLLGIVVAAGLALGTLVGFSTLVACLVCRKEKKTKGPSRHPSLISSDSNNLKLNNVRLPRENMSLPSNLQLNDLTPDSRAVKPADRQMAQNNSRPELLDPEPGGLLTSQGFIRLPVLGYIYRVSSVSSDEIWL

SEQ ID NO: 33 - protein, 257 aa, Homo sapiens
LNDLNVSPPELTVHVGDSALMGCVFQSTEDKCIFKIDWTLSPGEHAKDEYVLYYYSNLSVPIGRFQNRVHLMGDILCNDGSLLLQDVQEADQGTYICEIRLKGESQVFKKAVVLHVLPEEPKELMVHVGGLIQMGCVFQSTEVKHVTKVEWIFSGRRAKEEIVFRYYHKLRMSVEYSQSWGHFQNRVNLVGDIFRNDGSIMLQGVRESDGGNYTCSIHLGNLVFKKTIVLHVSPEEPRTLVTPAALRPLVLGGNQLV

SEQ ID NO: 34 - protein, 218 aa, Homo sapiens [sequence omitted]

SEQ ID NO: 35 - protein, 217 aa, Homo sapiens
THTEIKRVAEEKVTLPCHHQLGLPEKDTLDIEWLLTDNEGNQKVVITYSSRHVYNNLTEEQKGRVAFASNFLAGDASLQIEPLKPSDEGRYTCKVKNSGRYVWSHVILKVLVRPSKPKCELEGELTEGSDLTLQCESSSGTEPIVYYWQRIREKEGEDERLPPKSRIDYNHPGRVLLQNLTMSYSGLYQCTAGNEAGKESCVVRVTVQYVQSIGMVA

SEQ ID NO: 36 - protein, 219 aa, Homo sapiens
QLQLHLPANRLQAVEGGEVVLPAWYTLHGEVSSSQPWEVPFVMWFFKQKEKEDQVLSYINGVTTSKPGVSLVYSMPSRNLSLRLEGLQEKDSGPYSCSVNVQDKQGKSRGHSIKTLELNVLVPPAPPSCRLQGVPHVGANVTLSCQSPRSKPAVQYQWDRQLPSFQTFFAPALDVIRGSLSLTNLSSSMAGVYVCKAHNEVGTAQCNVTLEVSTGPGAA

SEQ ID NO: 37 - protein, 214 aa, Homo sapiens
ISVETPQDVLRASQGKSVTLPCTYHTSTSSREGLIQWDKLLLTHTERVVIWPFSNKNYIHGELYKNRVSISNNAEQSDASITIDQLTMADNGTYECSVSLMSDLEGNTKSRVRLLVLVPPSKPECGIEGETIIGNNIQLTCQSKEGSPTPQYSWKRYNILNQEQPLAQPASGQPVSLKNISTDTSGYYICTSSNEEGTQFCNITVAVRSPSMNV

SEQ ID NO: 38 - protein, 211 aa, Homo sapiens
SVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGV

SEQ ID NO: 39 - protein, 210 aa, Homo sapiens
FSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLNIS

SEQ ID NO: 40 - protein, 210 aa, Homo sapiens
VNLKSSNRTPVVQEFESVELSCIITDSQTSDPRIEWKKIQDEQTTYVFFDNKIQGDLAGRAEILGKTSLKIWNVTRRDSALYRCEVVARNDRKEIDEIVIELTVQVKPVTPVCRVPKAVPVGKMATLHCQESEGHPRPHYSWYRNDVPLPTDSRANPRFRNSSFHLNSETGTLVFTAVHKDDSGQYYCIASNDAGSARCEEQEMEVYDLN

SEQ ID NO: 41 - protein, 215 aa, Homo sapiens
QVSVVQVTIPDGFVNVTVGSNVTLICIYTTTVASREQLSIQWSFFHKKEMEPISHSSCLSTEGMEEKAVSQCLKMTHARDARGRCSWTSEIYFSQGGQAVAIGQFKDRITGSNDPGNASITISHMQPADSGIYICDVNNPPDFLGQNQGILNVSVLVKPSKPLCSVQGRPETGHTISLSCLSALGTPSPVYYWHKLEGRDIVPVKENFNPTTGIL

SEQ ID NO: 42 - protein, 220 aa, Homo sapiens
VEVKVPTEPLSTPLGKTAELTCTYSTSVGDSFALEWSFVQPGKPISESHPILYFTNGHLYPTGSKSKRVSLLQNPPTVGVATLKLTDVHPSDTGTYLCQVNNPPDFYTNGLGLINLTVLVPPSNPLCSQSGQTSVGGSTALRCSSSEGAPKPVYNWVRLGTFPTPSPGSMVQDEVSGQLILTNLSLTSSGTYRCVATNQMGSASCELTLSVTEPSQGRVA

SEQ ID NO: 43 - protein, 222 aa, Homo sapiens
ASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGL

SEQ ID NO: 44 - protein, 264 aa, Homo sapiens
RPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLP

SEQ ID NO: 45 - protein, 242 aa, Homo sapiens
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIG

SEQ ID NO: 46 - protein, 330 aa, Homo sapiens
QNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDH

SEQ ID NO: 47 - protein, 343 aa, Homo sapiens
QFPLTQNVTVVEGGTAILTCRVDQNDNTSLQWSNPAQQTLYFDDKKALRDNRIELVRASWHELSISVSDVSLSDEGQYTCSLFTMPVKTSKAYLTVLGVPEKPQISGFSSPVMEGDLMQLTCKTSGSKPAADIRWFKNDKEIKDVKYLKEEDANRKTFTVSSTLDFRVDRSDDGVAVICRVDHESLNATPQVAMQVLEIHYTPSVKIIPSTPFPQEGQPLILTCESKGKPLPEPVLWTKDGGELPDPDRMVVSGRELNILFLNKTDNGTYRCEATNTIGQSSAEYVLIVHDVPNTLLPTTIIPSLTTATVTTTVAITTSPTTSATTSSIRDPNALAGQNGPDH

SEQ ID NO: 48 - protein, 306 aa, Homo sapiens
NLSQDGYWQEQDLELGTLAPLDEAISSTVWSSPDMLASQDSQPWTSDETVVAGGTVVLKCQVKDHEDSSLQWSNPAQQTLYFGEKRALRDNRIQLVTSTPHELSISISNVALADEGEYTCSIFTMPVRTAKSLVTVLGIPQKPIITGYKSSLREKDTATLNCQSSGSKPAARLTWRKGDQELHGEPTRIQEDPNGKTFTVSSSVTFQVTREDDGASIVCSVNHESLKGADRSTSQRIEVLYTPTAMIRPDPPHPREGQKLLLHCEGRGNPVPQQYLWEKEGSVPPLKMTQESALIFPFLNKSDSGT

SEQ ID NO: 49 - protein, 300 aa, Homo sapiens
QEVQTENVTVAEGGVAEITCRLHQYDGSIVVIQNPARQTLFFNGTRALKDERFQLEEFSPRRVRIRLSDARLEDEGGYFCQLYTEDTHHQIATLTVLVAPENPVVEVREQAVEGGEVELSCLVPRSRPAATLRWYRDRKELKGVSSSQENGKVWSVASTVRFRVDRKDDGGIIICEAQNQALPSGHSKQTQYVLDVQYSPTARIHASQAVVREGDTLVLTCAVTGNPRPNQIRWNRGNESLPERAEAVGETLTLPGLVSADNGTYTCEASNKHGHARALYVLVVYDPGAVVEAQTSVPYA

SEQ ID NO: 50 - protein, 270 aa, Homo sapiens
SLTNHTETITVEEGQTLTLKCVTSLRKNSSLQWLTPSGFTIFLNEYPALKNSKYQLLHHSANQLSITVPNVTLQDEGVYKCLHYSDSVSTKEVKVIVLATPFKPILEASVIRKQNGEEHVVLMCSTMRSKPPPQITWLLGNSMEVSGGTLHEFETDGKKCNTTSTLIIHTYGKNSTVDCIIRHRGLQGRKLVAPFRFEDLVTDEETASDALERNSLSSQDPQQPTSTVSVTEDSSTSEIDKEEKEQTTQDPDLTTEANPQYLGLARKKSG

SEQ ID NO: 51 - protein, 191 aa, Homo sapiens
VLTVNGKTENYILDTTPGSQASLICAVQNHTREEELLWYREEGRVDLKSGNKINSSSVCVSSISENDNGISFTCRLGRDQSVSVSVVLNVTFPPLLSGNDFQTVEEGSNVKLVCNVKANPQAQMMWYKNSSLLDLEKSRHQIQQTSESFQLSITKVEKPDNGTYSCIAKSSLKTESLDFHLIVKDKTVGVP

SEQ ID NO: 52 - protein, 496 aa, Homo sapiens
KTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMS

SEQ ID NO: 53 - protein, 202 aa, Homo sapiens
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKG

SEQ ID NO: 54 - protein, 219 aa, Homo sapiens
EGAAQPNNSLMLQTSKENHALASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSH

SEQ ID NO: 55 - protein, 220 aa, Homo sapiens
SCMGGKQMTQNYSTIFAEGNISQPVLMDINAVLCCPPIALRNLIIITWEIILRGQPSCTKAYKKETNETKETNCTVERITWVSRPDQNSDLQIRPVDTTHDGYYRGIVVTPDGNFHRGYHLQVLVTPEVNLFQSRNITAVCKAVTGKPAAQISWIPEGSILATKQEYWGNGTVTVKSTCPWEGHKSTVTCHVSHLTGNKSLSVKLNSGLRTSGSPALSLL

SEQ ID NO: 56 - protein, 236 aa, Homo sapiens [sequence omitted]

SEQ ID NO: 57 - protein, 132 aa, Homo sapiens
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADL

SEQ ID NO: 58 - protein, 316 aa, Homo sapiens
DVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGMSRN

SEQ ID NO: 59 - protein, 325 aa, Homo sapiens
QVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGTYICEATNPIGTRSGQVEVNITEFPYTPSPPEHGRRAGPVPTA

SEQ ID NO: 60 - protein, 329 aa, Homo sapiens
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 61 - protein, 347 aa, Homo sapiens
GPIIVEPHVTAVWGKNVSLKCLIEVNETITQISWEKIHGKSSQTVAVHHPQYGFSVQGEYQGRVLFKNYSLNDATITLHNIGFSDSGKYICKAVTFPLGNAQSSTTVTVLVEPTVSLIKGPDSLIDGGNETVAAICIAATGKPVAHIDWEGDLGEMESTTTSFPNETATIISQYKLFPTRFARGRRITCVVKHPALEKDIRYSFILDIQYAPEVSVTGYDGNWFVGRKGVNLKCNADANPPPFKSVWSRLDGQWPDGLLASDNTLHFVHPLTFNYSGVYICKVTNSLGQRSDQKVIYISDPPTTTTLQPTIQWHPSTADIEDLATEPKKLPFPLSTLATIKDDTIAT

SEQ ID NO: 62 - protein, 318 aa, Homo sapiens
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSAS

SEQ ID NO: 63 - protein, 141 aa, Homo sapiens
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVA

SEQ ID NO: 64 - protein, 206 aa, Homo sapiens
ELEPQIDGQTWAERALRENERHAFTCRVAGGPGTPRLAWYLDGQLQEASTSRLLSVGGEAFSGGTSTFTVTAHRAQHELNCSLQDPRSGRSANASVILNVQFKPEIAQVGAKYQEAQGPGLLVVLFALVRANPPANVTWIDQDGPVTVNTSDFLVLDAQNYPWLTNHTVQLQLRSLAHNLSVVATNDVGVTSASLPAPGLLATRVE

SEQ ID NO: 65 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 230 aa, Homo sapiens
KSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 321 aa, Artificial Sequence
  dnYAP
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHASPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNLNDMESVLAATKLDKESFLTWL

SEQ ID NO: 69 - protein, 239 aa, Artificial Sequence
  YFP
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 70 - protein, 447 aa, Homo sapiens
MGEPRAGAALDDGSGWTGSEEGSEEGTGGSEGAGGDGGPDAEGVWSPDIEQSFQEALAIYPPCGRRKIILSDEGKMYGRNELIARYIKLRTGKTRTRKQVSSHIQVLARRKSREIQSKLKDQVSKDKAFQTMATMSSAQLISAPSLQAKLGPTGPQASELFQFWSGGSGPPWNVPDVKPFSQTPFTLSLTPPSTDLPGYEPPQALSPLPPPTPSPPAWQARGLGTARLQLVEFSAFVEPPDAVDSYQRHLFVHISQHCPSPGAPPLESVDVRQIYDKFPEKKGGLRELYDRGPPHAFFLVKFWADLNWGPSGEEAGAGGSISSGGFYGVSSQYESLEHMTLTCSSKVCSFGKQVVEKVETERAQLEDGRFVYRLLRSPMCEYLVNFLHKLRQLPERYMMNSVLENFTILQVVTNRDTQELLLCTAYVFEVSTSERGAQHHIYRLVRD

SEQ ID NO: 71 - protein, 435 aa, Homo sapiens
MASNSWNASSSPGEAREDGPEGLDKGLDNDAEGVWSPDIEQSFQEALAIYPPCGRRKIILSDEGKMYGRNELIARYIKLRTGKTRTRKQVSSHIQVLARKKVREYQVGIKAMNLDQVSKDKALQSMASMSSAQIVSASVLQNKFSPPSPLPQAVFSTSSRFWSSPPLLGQQPGPSQDIKPFAQPAYPIQPPLPPTLSSYEPLAPLPSAAASVPVWQDRTIASSRLRLLEYSAFMEVQRDPDTYSKHLFVHIGQTNPAFSDPPLEAVDVRQIYDKFPEKKGGLKELYEKGPPNAFFLVKFWADLNSTIQEGPGAFYGVSSQYSSADSMTISVSTKVCSFGKQVVEKVETEYARLENGRFVYRIHRSPMCEYMINFIHKLKHLPEKYMMNSVLENFTILQVVTSRDSQETLLVIAFVFEVSTSEHGAQHHVYKLVKD

SEQ ID NO: 72 - protein, 17 aa, Artificial Sequence
  Linker
RPACKIPNDLKQKVMNH

SEQ ID NO: 73 - protein, 8 aa, Artificial Sequence
  FLAG Tag
DYKDDDDK

SEQ ID NO: 74 - protein, 8 aa, Artificial Sequence
  Linker
ESGGGGVT

SEQ ID NO: 75 - protein, 9 aa, Artificial Sequence
  Linker
LESGGGGVT

SEQ ID NO: 76 - protein, 6 aa, Artificial Sequence
  Linker
GRAQVT

SEQ ID NO: 77 - protein, 6 aa, Artificial Sequence
  Linker
WRAQVT

SEQ ID NO: 78 - protein, 8 aa, Artificial Sequence
  Linker
ARGRAQVT

SEQ ID NO: 79 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2014/0056890 A1
(43) Pub. Date: Feb. 27, 2014

(54) Binding Agents That Modulate the Hippo Pathway and Uses Thereof

(71) Applicant: OncoMed Pharmaceuticals, Inc., Redwood City, CA (US)

(72) Inventors: Austin L. GURNEY, San Francisco, CA (US); Cecile Chartier-Courtaud, Palo Alto, CA (US)

(21) Appl. No.: 13/911,756

(22) Filed: Jun. 6, 2013

Related U.S. Application Data
(60) Provisional application No. 61/656,249, filed on Jun. 6, 2012.
(60) Provisional application No. 61/737,390, filed on Dec. 14, 2012.
(60) Provisional application No. 61/783,190, filed on Mar. 14, 2013.

(51) Int. Cl. C07K 16/28; A61K 45/06; A61K 39/395

(52) U.S. Cl. CPC C07K 16/2803; A61K 39/3955; A61K 45/06
USPC 424/134.1; 530/387.3; 435/328; 536/23.53; 435/375

(57) ABSTRACT

The present invention relates to agents that modulate the Hippo pathway and Hippo pathway signaling, such as antibodies and soluble receptors, as well as to methods of using the agents for the treatment of diseases such as cancer.

### CROSS-REFERENCE TO RELATED APPLICATIONS

This application claims priority benefit of U.S. Provisional Application No. 61/656,249, filed Jun. 6, 2012, U.S. Provisional Application No. 61/737,390, filed Dec. 14, 2012, and U.S. Provisional Application No. 61/783,190, filed Mar. 14, 2013, each of which is hereby incorporated by reference herein in its entirety.

### FIELD OF THE INVENTION

This invention generally relates to agents that modulate the Hippo pathway and Hippo pathway signaling, such as antibodies and soluble receptors, as well as to methods of using the agents for the treatment of diseases such as cancer.

### BACKGROUND OF THE INVENTION

The Hippo pathway is a signaling pathway that regulates cell proliferation and cell death and determines organ size. The pathway is believed to play a role as a tumor suppressor in mammals and disorders of the pathway are often detected in human cancers. The pathway is involved in and/or may regulate the self-renewal and differentiation of stem cells and progenitor cells. In addition, the Hippo pathway may be involved in wound healing and tissue regeneration. Furthermore, it is believed that as the Hippo pathway cross-talks with other signaling pathways such as Wnt, Notch, and Hedgehog, it may influence a wide variety of biological events and that its dysfunction could be involved in many human diseases in addition to cancer. For reviews, see, for example, Halder et al., 2011, Development 138:9-22; Zhao et al., 2011, Nature Cell Biology 13:877-883; Bao et al., 2011, J. Biochem. 149:361-379; Zhao at al., 2010, J. Cell Sci. 123:4001-4006.

The Hippo pathway is composed of a kinase cascade that is highly conserved across Drosophila and mammalian systems. In Drosophilia, the atypical cadherins, Dachsous (Ds) and Fat (Ft) have been reported to function as receptors for the Hippo pathway (see, for example, Bennett at al., 2006, Curr. Biol., 16:2101-2110; Matakatsu at al., 2006, Development, 133:2315-2324). Recently, it was reported that the Drosophila cell adhesion molecule Echinoid (Ed) interacts with Salvador (Say) and may be involved in Hippo signaling (Yue at al, 2012, Dev. Cell, 22:255-267). However, a cell surface receptor has not been identified for the mammalian Hippo pathway.

Two of the core components of the mammalian Hippo pathway are Lats1 and Lats2, which are nuclear Dbf2-related (NDR) family protein kinases homologous to Drosophila Warts (Wts). The Lats1/2 proteins are activated by association with the scaffold proteins Mob1A/B (Mps one binder kinase activator-like 1A and 1B), which are homologous to Drosophila Mats. Lats1/2 proteins are also activated by phosphorylation by the STE20 family protein kinases Mst1 and Mst2, which are homologous to Drosophila Hippo. Lats1/2 kinases phosphorylate the downstream effectors YAP (Yes-associated protein) and TAZ (transcriptional coactivator with PDZ-binding motif; WWTR1), which are homologous to Drosophila Yorkie. The phosphorylation of YAP and TAZ by Lats1/2 are crucial events within the Hippo signaling pathway. Lats1/2 phosphorylates YAP at multiple sites, but phosphorylation of Ser127 is critical for YAP inhibition. Phosphorylation of YAP generates a protein-binding motif for the 14-3-3 family of proteins, which upon binding of a 14-3-3 protein, leads to retention and/or sequestration of YAP in the cell cytoplasm. Likewise, Lats1/2 phosphorylates TAZ at multiple sites, but phosphorylation of Ser89 is critical for TAZ inhibition. Phosphorylation of TAZ leads to retention and/or sequestration of TAZ in the cell cytoplasm. In addition, phosphorylation of YAP and TAZ is believed to destabilize these proteins by activating phosphorylation-dependent degradation catalyzed by YAP or TAZ ubiquitination. Thus, when the Hippo pathway is “on”, YAP and/or TAZ is phosphorylated, inactive, and generally sequestered in the cytoplasm; in contrast, when the Hippo pathway is “off”, YAP and/or TAZ is non-phosphorylated, active, and generally found in the nucleus.

Non-phosphorylated, activated YAP is translocated into the cell nucleus where its major target transcription factors are the four proteins of the TEA-domain-containing family (TEAD1-TEAD4, collectively “TEAD”). YAP together with TEAD (or other transcription factors such as Smad1, RUNX, ErbB4 and p73) has been shown to induce the expression of a variety of genes, including connective tissue growth factor (CTGF), Gli2, Birc5, Birc2, fibroblast growth factor 1 (FGF1), and amphiregulin (AREG). Like YAP, non-phosphorylated TAZ is translocated into the cell nucleus where it interacts with multiple DNA-binding transcription factors, such as peroxisome proliferator-activated receptor γ (PPARγ), thyroid transcription factor-1 (TTF-1), Pax3, TBX5, RUNX, TEAD1 and Smad2/3/4. Many of the genes activated by YAP/TAZ-transcription factor complexes mediate cell survival and proliferation. Therefore, under some conditions YAP and/or TAZ acts as an oncogene and the Hippo pathway acts as a tumor suppressor. Thus, targeting the Hippo pathway may be another area for therapeutic intervention for cancer and other diseases.

### BRIEF SUMMARY OF THE INVENTION

The present invention provides binding agents, such as antibodies, soluble receptors, and small molecules that modulate the Hippo pathway. The invention also provides compositions, such as pharmaceutical compositions, comprising the binding agents. In certain embodiments, the binding agents that modulate the Hippo pathway are novel polypeptides, such as antibodies and fusion proteins. In certain embodiments, the binding agents that modulate the Hippo pathway are novel small peptides or small molecules. In certain embodiments, the binding agents are antibodies or fragments thereof that specifically bind human cell adhesion molecules of the immunoglobulin superfamily (IgCAM). The invention further provides methods of inhibiting the growth of a tumor by administering the binding agents that modulate the Hippo pathway to a subject with a tumor. The invention further provides methods of treating cancer by administering the binding agents that modulate the Hippo pathway to a subject in need thereof. In some embodiments, the methods of treating cancer or inhibiting tumor growth comprise activating or stimulating the Hippo pathway. In some embodiments, the methods of treating cancer or inhibiting tumor growth comprise inhibiting YAP activity. In some embodiments, the methods of treating cancer or inhibiting tumor growth comprise inhibiting TAZ activity. In certain embodiments, the methods comprise reducing the tumorigenicity of a tumor.

In one aspect, the invention provides a binding agent, such as an antibody or a soluble receptor, that specifically binds one or more human IgCAMs. These binding agents are referred to herein as “IgCAM-binding agents”. In some embodiments, the binding agent specifically binds the extracellular domain of at least one human IgCAM. In some embodiments, the binding agent specifically binds the extracellular domain of at least one human IgCAM selected from the JAM (junctional adhesion molecule) family, the PVR (poliovirus receptor) family, and/or the CADM (cell adhesion molecule) family. In some embodiments, the binding agent specifically binds the extracellular domain of one or more IgCAMs selected from the group consisting of: AMICA, CAR, CLMP, ESAM, GPA33, VSIG1, VSIG2, VSIG3. VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In some embodiments, the binding agent specifically binds the extracellular domain of CLMP, VSIG4, VSIG8, JAM2, JAM3, CADM1, CADM3, CADM4, PVRL1, PVRL3, PVRL4, TIGIT, TMIGD1, and/or TMEM25. In some embodiments, the binding agent specifically binds the extracellular domain of CADM1, CADM3, PVRL1 and/or PVRL3. In some embodiments, the binding agent specifically binds the extracellular domain of CADM3, PVRL1 and/or PVRL3. In some embodiments, the binding agent specifically binds the extracellular domain of CADM3.

In some embodiments, the IgCAM-binding agent modulates Hippo pathway activity. In some embodiments, the IgCAM-binding agent increases Hippo pathway signaling. In some embodiments, the IgCAM-binding agent is an agonist of Hippo pathway activity and/or enhances Hippo pathway signaling. In some embodiments, the IgCAM-binding agent inhibits YAP activity, enhances phosphorylation of YAP, enhances degradation of YAP, and/or inhibits activation of YAP. In some embodiments, the IgCAM-binding agent inhibits TAZ activity, enhances phosphorylation of TAZ, enhances degradation of TAZ, and/or inhibits activation of TAZ.

In some embodiments, the IgCAM-binding agent inhibits Hippo pathway signaling. In some embodiments, the IgCAM-binding agent is an antagonist of Hippo pathway activity and/or decreases Hippo pathway signaling. In some embodiments, the IgCAM-binding agent increases YAP activity, decreases phosphorylation of YAP, decreases degradation of YAP, and/or increases activation of YAP. In some embodiments, the IgCAM-binding agent increases TAZ activity, decreases phosphorylation of TAZ, decreases degradation of TAZ, and/or increases activation of TAZ.

In certain embodiments, the IgCAM-binding agent is a polypeptide. In certain embodiments, the IgCAM-binding agent is isolated. In certain embodiments, the IgCAM-binding agent is substantially pure.

In some embodiments of each of the aforementioned aspects and embodiments, as well as other aspects and embodiments described herein, the IgCAM-binding agent is an antibody. In some embodiments, the antibody is a monoclonal antibody. In some embodiments, the antibody is a monovalent antibody. In some embodiments, the antibody is a bispecific antibody. In some embodiments, the antibody is an antibody fragment. In some embodiments, the antibody binds at least one human IgCAM. In some embodiments, the antibody binds at least one human IgCAM and at least one mouse IgCAM. In some embodiments, the antibody binds at least one IgCAM with a K_{D} of less than 10 nM.

In certain embodiments of each of the aforementioned aspects and embodiments, as well as other aspects and embodiments described herein, the IgCAM-binding agent is a soluble receptor. In some embodiments, the soluble receptor comprises the extracellular domain of an IgCAM. In some embodiments, the soluble receptor comprises a portion of the extracellular domain of an IgCAM. In some embodiments, the soluble receptor is a fusion protein. In some embodiments, the soluble receptor comprises a human Fc region. In some embodiments, the soluble receptor binds at least one human IgCAM. In some embodiments, the soluble receptor binds at least one human IgCAM and at least one mouse IgCAM. In some embodiments, the soluble receptor binds at least one IgCAM with a K_{D} of less than 10 nM.

In another aspect, the invention provides isolated polynucleotide molecules comprising a polynucleotide that encodes a binding agent of the invention. The invention further provides expression vectors that comprise these polynucleotides, as well as cells that comprise the expression vectors and/or the polynucleotides. In some embodiments, the cell is a hybridoma cell line.

In other aspects, the invention provides methods of inhibiting growth of a tumor, comprising contacting the tumor with an effective amount of an IgCAM-binding agent that modulates the Hippo pathway, including each of the binding agents described herein. In some embodiments, the IgCAM-binding agent is an agonist of Hippo pathway activity, enhances Hippo pathway signaling, inhibits YAP activity, enhances phosphorylation of YAP, enhances degradation of YAP, inhibits activation of YAP, inhibits TAZ activity, enhances phosphorylation of TAZ, enhances degradation of TAZ, and/or inhibits activation of TAZ.

In another aspect, the invention provides a method of inhibiting the growth of a tumor in a subject, comprising administering to the subject a therapeutically effective amount of an IgCAM-binding agent that modulates the Hippo pathway, including each of the binding agents described herein. In some embodiments, the IgCAM-binding agent is an agonist of Hippo pathway activity, enhances Hippo pathway signaling, inhibits YAP activity, enhances phosphorylation of YAP, enhances degradation of YAP, inhibits activation of YAP, inhibits TAZ activity, enhances phosphorylation of TAZ, enhances degradation of TAZ, and/or inhibits activation of TAZ.

In another aspect, the invention provides a method of modulating Hippo pathway signaling in a cell, comprising contacting the cell with an effective amount of an IgCAM-binding agent, including any of those described herein. In some embodiments, the cell is a tumor cell. In some embodiments, the tumor is a colorectal tumor. In some embodiments, the tumor is a breast tumor. In some embodiments, the tumor is an ovarian tumor. In some embodiments, the tumor is a pancreatic tumor. In some embodiments, the tumor is a lung tumor. In some embodiments, the tumor is a melanoma tumor. In some embodiments, the tumor expresses elevated levels of YAP. In some embodiments, the tumor expresses elevated levels of non-phosphorylated YAP. In some embodiments, the tumor contains elevated levels of YAP in the cell nucleus. In some embodiments, the tumor has elevated expression levels of YAP-dependent genes. In some embodiments, the tumor expresses elevated levels of TAZ. In some embodiments, the tumor expresses elevated levels of non-phosphorylated TAZ. In some embodiments, the tumor contains elevated levels of TAZ in the cell nucleus. In some embodiments, the tumor has elevated expression levels of TAZ-dependent genes. In certain embodiments, the IgCAM-binding agent inhibits growth of the tumor.

In another aspect, the invention provides methods of treating cancer in a subject. In some embodiments, the method comprises administering to a subject a therapeutically effective amount of any of the binding agents that modulate Hippo pathway signaling described herein. In some embodiments, the binding agent is an agonist of Hippo pathway activity, enhances Hippo pathway signaling, inhibits YAP activity, enhances phosphorylation of YAP, enhances degradation of YAP, and/or inhibits activation of YAP. In some embodiments, the binding agent inhibits TAZ activity, enhances phosphorylation of TAZ, enhances degradation of TAZ, and/or inhibits activation of TAZ. In some embodiments, the cancer is pancreatic cancer. In some embodiments, the cancer is colorectal cancer. In some embodiments, the cancer is breast cancer. In some embodiments, the cancer is lung cancer. In some embodiments, the cancer is melanoma. In some embodiments, the cancer is ovarian cancer. In some embodiments, the cancer expresses elevated levels of YAP. In some embodiments, the cancer expresses elevated levels of non-phosphorylated YAP. In some embodiments, the cancer has elevated expression levels of YAP-dependent genes. In some embodiments, the cancer expresses elevated levels of TAZ. In some embodiments, the cancer expresses elevated levels of non-phosphorylated TAZ. In some embodiments, the cancer has elevated expression levels of TAZ-dependent genes.

In another aspect, the invention provides methods of treating a disease in a subject wherein the disease is associated with activation of YAP and/or aberrant YAP activity, wherein the methods comprise administering a therapeutically effective amount of a binding agent that modulates the Hippo pathway, including any of the binding agents described herein. In some embodiments, the modulation of the Hippo pathway comprises increasing Hippo pathway signaling. In some embodiments, the modulation of the Hippo pathway comprises increasing YAP phosphorylation. In some embodiments, the modulation of the Hippo pathway comprises increasing YAP degradation. In some embodiments, the modulation of the Hippo pathway comprises increasing retention of YAP in the cytoplasm. In some embodiments, the modulation of the Hippo pathway comprises reducing YAP translocation to the nucleus. In some embodiments, the modulation of the Hippo pathway comprises reducing the expression of YAP-dependent genes.

In another aspect, the invention provides methods of treating a disease in a subject wherein the disease is associated with activation of TAZ and/or aberrant TAZ activity, wherein the methods comprise administering a therapeutically effective amount of a binding agent that modulates the Hippo pathway, including each of the binding agents described herein. In some embodiments, the modulation of the Hippo pathway comprises increasing Hippo pathway signaling. In some embodiments, the modulation of the Hippo pathway comprises increasing TAZ phosphorylation. In some embodiments, the modulation of the Hippo pathway comprises increasing TAZ degradation. In some embodiments, the modulation of the Hippo pathway comprises increasing retention of TAZ in the cytoplasm. In some embodiments, the modulation of the Hippo pathway comprises reducing TAZ translocation to the nucleus. In some embodiments, the modulation of the Hippo pathway comprises reducing the expression of TAZ-dependent genes.

In some embodiments, invention provides a method of modulating Hippo pathway signaling in a cell, comprising contacting the cell with an effective amount of an IgCAM-binding agent that inhibits Hippo pathway signaling. In some embodiments, the modulation of the Hippo pathway comprises decreasing Hippo pathway signaling. In some embodiments, the modulation of the Hippo pathway comprises decreasing YAP phosphorylation. In some embodiments, the modulation of the Hippo pathway comprises decreasing YAP degradation. In some embodiments, the modulation of the Hippo pathway comprises decreasing retention of YAP in the cytoplasm. In some embodiments, the modulation of the Hippo pathway comprises increasing YAP translocation to the nucleus. In some embodiments, the modulation of the Hippo pathway comprises increasing the expression of YAP-dependent genes. In some embodiments, the modulation of the Hippo pathway comprises decreasing TAZ phosphorylation. In some embodiments, the modulation of the Hippo pathway comprises decreasing TAZ degradation. In some embodiments, the modulation of the Hippo pathway comprises decreasing retention of TAZ in the cytoplasm. In some embodiments, the modulation of the Hippo pathway comprises increasing TAZ translocation to the nucleus. In some embodiments, the modulation of the Hippo pathway comprises increasing the expression of TAZ-dependent genes.

In another aspect, the invention provides methods of treating a wound. In some embodiments the methods promote and/or enhance wound healing in a subject. In some embodiments, the methods comprise administering to a subject a therapeutically effective amount of a binding agent that modulates Hippo pathway signaling, such as a binding agent described herein. In some embodiments, the administered binding agent is an antagonist of Hippo pathway activity, suppresses Hippo pathway signaling, increases YAP activity, suppresses phosphorylation of YAP, suppresses degradation of YAP, and/or promotes activation of YAP. In some embodiments, the administered binding agent increases TAZ activity, suppresses phosphorylation of TAZ, suppresses degradation of TAZ, and/or promotes activation of TAZ. In some embodiments, the wound is an acute cutaneous wound. In some embodiments, the wound is a chronic cutaneous wound. In some embodiments, the wound is a surgical wound.

In another aspect, the invention provides methods of enhancing tissue regeneration. In some embodiments, the methods comprise contacting cells with an effective amount of a binding agent that modulates Hippo pathway signaling, such as a binding agent described herein. In some embodiments, the methods comprise administering to a subject a therapeutically effective amount of a binding agent that modulates Hippo pathway signaling, such as a binding agent described herein. In some embodiments, the modulation of Hippo pathway signaling comprises antagonizing the Hippo pathway. In some embodiments, the modulation of Hippo pathway signaling comprises reducing signaling of the Hippo pathway. In some embodiments, the method involves the treatment of tissue damage caused by immune related disorders (such as autoimmune disorders); inflammation (including both acute and chronic inflammatory disorders); ischemia (such as myocardial infarction); traumatic injury (such as burns, lacerations, and abrasions); infection (such as bacterial, viral, and fungal infections); and chronic damage (such as cirrhosis of the liver).

In certain embodiments of each of the aforementioned aspects, as well as other aspects and/or embodiments described elsewhere herein, the methods comprise a step of determining the location of YAP and/or TAZ (e.g., nuclear or cytoplasmic) in the cells of the tumor or cancer. In certain embodiments, of each of the aforementioned aspects, as well as other aspects and/or embodiments described elsewhere herein, the methods comprise a step of determining the expression level of YAP-dependent genes or proteins encoded by these genes. In certain embodiments, the methods comprise a step of determining the expression level of TAZ-dependent genes or proteins encoded by these genes.

In another aspect, the invention provides a method of identifying a human subject or selecting a human subject for treatment with a binding agent that modulates Hippo pathway signaling, including but not limited to, each of the binding agents described herein. In some embodiments, the method comprises determining if the subject has a tumor that has an elevated expression level of YAP as compared to the expression of YAP in a reference sample. In some embodiments, the method comprises determining if the subject has a tumor that has an elevated expression level of YAP as compared to a pre-determined level of YAP. In some embodiments, the method comprises determining if the subject has a tumor that has an elevated expression level of YAP as compared to the expression of YAP in normal tissue. In some embodiments, the method comprises determining if the subject has a tumor that has an elevated level of YAP in the nucleus of tumor cells as compared to the level of YAP in the nucleus of cells from normal tissue. In some embodiments, the method comprises determining if the subject has a tumor that has an elevated level of non-phosphorylated YAP in tumor cells as compared to the level of non-phosphorylated YAP in cells from normal tissue. In some embodiments, the method comprises determining if the subject has a tumor that has an elevated expression level of TAZ as compared to the expression of TAZ in normal tissue. In some embodiments, the method comprises determining if the subject has a tumor that has an elevated level of TAZ in the nucleus of tumor cells as compared to the level of TAZ in the nucleus of cells from normal tissue. In some embodiments, the method comprises determining if the subject has a tumor that has an elevated level of non-phosphorylated TAZ in tumor cells as compared to the level of non-phosphorylated TAZ in cells from normal tissue. In some embodiments, the “normal tissue” is a tissue of the same type as the tumor, e.g., a lung tumor and normal lung tissue.

In another aspect, the invention provides methods of targeting tumor cells with an IgCAM-binding agent, such as the agents described herein. In some embodiments, the methods comprise administering to a subject an effective amount of an IgCAM-binding agent. In some embodiments, the binding agent is an antibody. In some embodiments, the IgCAM-binding agent comprises a soluble receptor. In some embodiments, the IgCAM-binding agent is a soluble receptor. In some embodiments, the IgCAM-binding agent is conjugated to or complexed with a cytotoxic agent.

Compositions comprising an IgCAM-binding agent as described herein and cell lines that produce the binding agents are provided. Moreover, pharmaceutical compositions comprising an IgCAM-binding agent described herein and a pharmaceutically acceptable carrier are further provided. Methods of treating cancer and/or inhibiting tumor growth in a subject comprising administering to the subject an effective amount of a composition comprising a binding agent such as the IgCAM-binding agents described herein are also provided. Methods of enhancing wound healing and/or tissue regeneration in a subject comprising administering to the subject an effective amount of a composition comprising a binding agent such as the IgCAM-binding agents described herein are also provided.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE FIGURES

FIG. 1. Microarray gene expression analysis of YAP in patient-derived tumors from the OncoMed tumor bank.

FIG. 2. Inhibition of tumor growth by the expression of dnYAP. Tumor cells were transduced with dnYAP-lentivirus (-∘-) or GFP-lentivirus (-•-) and incubated 72 hours. Transduced cells, identified as GFP positive and DAPI negative (GFP⁺/DAPI⁻), were sorted by flow cytometry and subcutaneously injected into NOD/SCID mice. Tumor growth was monitored for 3 months and data is shown as tumor volume (mm³). (A) OMP-M6 melanoma tumors; (B) OMP-C18 colon tumors; (C) OMP-C37 colon tumors; (D) OMP-PN7 pancreatic tumors; (E) OMP-C11 colon tumors; (F) OMP-L2 lung tumors; (G) OMP-OV22 ovarian tumors; (H) OMP-LU52 lung tumors. (I) YAP expression in tumors as determined by microarray analysis.

FIG. 3. Effect of expression of dnYAP on YAP activity.

FIG. 4. Effect of cell detachment on Hippo pathway components as determined by Western blot analysis.

FIG. 5. Family tree of candidate Hippo pathway receptors.

FIG. 6. Diagram of candidate Hippo pathway receptor, membrane-bound decoy receptor, and soluble receptor.

FIG. 7. Expression of membrane-anchored decoy receptor. (A) Cell surface expression of GFP-tagged IgCAM decoy receptor as viewed by fluorescent microscopy. (B) Flow cytometry histogram of GFP-tagged IgCAM decoy receptor.

FIG. 8. Effect of IgCAM decoy receptors on Hippo pathway components as determined by Western blot analysis.

FIG. 9. Bimolecular fluorescence complementation assay for determination of YAP nuclear translocation. (A) Diagrams of YFP constructs with YAP, TEAD2, and TEAD3. (B) Nuclear YFP fluorescence of low density cells as viewed by fluorescent microscopy and phase contrast.

FIG. 10. Tetracycline-inducible HeLa cell line NC12 expressing GFP-YAP. (A) Expression of GFP-YAP in response to Tet activation. (B) Western blot analysis of GFP-YAP expression in response to Tet activation.

FIG. 11. Diagrams of Cre-dependent EmGFP expression construct and YAP-Cre constructs for use in determination of YAP nuclear translocation.

FIG. 12. Effect on tumor growth by over-expression of membrane-anchored IgCAM decoy receptors in colon tumor OMP-C18. (A) CADM2, CADM4, CD226, PVR; (B) CADM3, TMEM25, JAM3, TIGIT; (C) CADM3, TMEM25, JAM3, TIGIT; (D) JAM2, PVRL1, ESAM; (E) VSIG1, VSIG4, PVRL4, CD200; (F) PVRL3, JAM1, CADM1, VSIG2; and (G) CLMP, VSIG8, TMIGD1, VSIG3.

FIG. 13. Effect on tumor growth by over-expression of membrane-anchored IgCAM decoy receptors in colon tumor OMP-LU2. (A) CADM2; (B) CADM3, PVRL4, VSIG4; (C) CD226, JAM2, JAM3, CADM1; (D) CADM4, TIGIT, PVRL1, PVRL3.

FIG. 14. Effect on tumor growth by over-expression of membrane-anchored IgCAM decoy receptors in colon tumor OMP-LU40. (A) CADM1, CADM2, CADM3, CADM4, CD226, PVR; (B) PVRL4, VSIG4, CD226; (C) PVRL3, ESAM, VSIG2.

FIG. 15. Western blot analysis of IgCAM protein complexes using cells transfected with FLAG tagged full-length CADM1, CADM3, PVR, PVRL1, and PVRL3. CADM3-CD4TM-GFP and LGR5-CD4TM-GFP constructs were used as negative controls. Protein complexes were analyzed for (A) Afadin, DCAF7, DLG1, INADL, LIN7, LIN7C; (B) MPDZ, MPP5?, PARD3, FLAG control; (C) YAP, phospho-YAP.

### DETAILED DESCRIPTION OF THE INVENTION

The present invention provides novel agents, including, but not limited to, polypeptides such as antibodies and soluble receptors that modulate the Hippo pathway. The agents include agonists and antagonists of Hippo pathway signaling. Related polypeptides and polynucleotides, compositions comprising the agents, and methods of making the agents are also provided. Methods of screening for agents that modulate the Hippo pathway are provided. Methods of using the novel agents, such as methods of inhibiting tumor growth, methods of treating cancer, methods of activating and/or enhancing Hippo pathway signaling, methods of inhibiting or reducing Hippo pathway signaling, and/or methods of identifying and/or selecting subjects for treatment, are further provided.

### I. DEFINITIONS

To facilitate an understanding of the present invention, a number of terms and phrases are defined below.

The terms “agonist” and “agonistic” as used herein refer to or describe an agent that is capable of, directly or indirectly, substantially inducing, activating, promoting, increasing, or enhancing the biological activity of a target and/or a signaling pathway (e.g., the Hippo pathway). The term “agonist” is used herein to include any agent that partially or fully induces, activates, promotes, increases, or enhances the activity of a protein. Suitable agonists specifically include, but are not limited to, agonist antibodies or fragments thereof, soluble receptors, and other fusion proteins.

The terms “antagonist” and “antagonistic” as used herein refer to or describe an agent that is capable of, directly or indirectly, partially or fully blocking, inhibiting, reducing, or neutralizing a biological activity of a target and/or signaling pathway (e.g., the Hippo pathway). The term “antagonist” is used herein to include any agent that partially or fully blocks, inhibits, reduces, or neutralizes the activity of a protein. Suitable antagonist agents specifically include, but are not limited to, antagonist antibodies or fragments thereof, soluble receptors, and other fusion proteins.

The terms “modulation” and “modulate” as used herein refer to a change or an alteration in a biological activity. Modulation includes, but is not limited to, stimulating or inhibiting an activity. Modulation may be an increase or a decrease in activity (e.g., a decrease in Hippo pathway signaling; an increase in Hippo pathway signaling), a change in binding characteristics, or any other change in the biological, functional, or immunological properties associated with the activity of a protein, pathway, or other biological point of interest.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies, multispecific antibodies such as bispecific antibodies generated from at least two intact antibodies, bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site.

The term “variable region” of an antibody refers to the variable region of the antibody light chain, or the variable region of the antibody heavy chain, either alone or in combination. The variable regions of the heavy and light chain each consist of four framework regions (FR) connected by three complementarity determining regions (CDRs), also known as “hypervariable regions”. The CDRs in each chain are held together in close proximity by the framework regions and, with the CDRs from the other chain, contribute to the formation of the antigen-binding sites of the antibody. There are at least two techniques for determining CDRs: (I) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen recognition site (antigen-binding site). Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability (Jones at al., 1986, Nature, 321:522-525; Riechmann et al., 1988, Nature, 332:323-327; Verhoeyen et al., 1988, Science, 239:1534-1536). In some instances, the Fv framework region residues of a human immunoglobulin are replaced with the corresponding residues in an antibody from a non-human species that has the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The humanized antibody may comprise substantially all of at least one, and typically two or three, variable domains containing all or substantially all of the CDRs that correspond to the non-human immunoglobulin whereas all or substantially all of the framework regions are those of a human immunoglobulin consensus sequence. The humanized antibody can also comprise at least a portion of an immunoglobulin constant region or domain (Fc), typically that of a human immunoglobulin.

The term “human antibody” as used herein refers to an antibody produced by a human or an antibody having an amino acid sequence corresponding to an antibody produced by a human made using any of the techniques known in the art. This definition of a human antibody specifically excludes a humanized antibody comprising non-human antigen-binding residues.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human) to avoid eliciting an immune response in that species.

The phrase “affinity matured antibody” as used herein refers to an antibody with one or more alterations in one or more CDRs thereof that result in an improvement in the affinity of the antibody for antigen as compared to a parent antibody that does not possess those alterations(s). Preferred affinity matured antibodies will have nanomolar or even picomolar affinities for the target antigen. Affinity matured antibodies are produced by procedures known in the art. For example, Marks et al., 1992, Bio/Technology 10:779-783, describes affinity maturation by VH and VL domain shuffling. Random mutagenesis of CDR and/or framework residues is described by Barbas et al., 1994, PNAS, 91:3809-3813; Schier et al., 1995, Gene, 169:147-155; Yelton et al., 1995, J. Immunol. 155:1994-2004; Jackson et al., 1995, J. Immunol., 154:3310-9; and Hawkins et al., 1992, J. Mol. Biol., 226:889-896.

As used herein the term “soluble receptor” refers to an extracellular fragment of a receptor protein that can be secreted from a cell in soluble form. The term “soluble receptor” encompasses a molecule comprising the entire extracellular domain, or a portion of the extracellular domain.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The terms “selectively binds” or “specifically binds” mean that a binding agent reacts or associates more frequently, more rapidly, with greater duration, with greater affinity, or with some combination of the above to the epitope, protein, or target molecule than with alternative substances, including unrelated proteins. In certain embodiments “specifically binds” means, for instance, that a binding agent binds a protein or target with a K_{D} of about 0.1 mM or less, but more usually less than about 1 μM. In certain embodiments, “specifically binds” means that a binding agent binds a target with a K_{D} of at least about 0.1 μM or less, at least about 0.01 μM or less, or at least about 1 nM or less. Because of the sequence identity between homologous proteins in different species, specific binding can include a binding agent that recognizes a protein or target in more than one species. Likewise, because of homology within certain regions of polypeptide sequences of different proteins, specific binding can include a binding agent that recognizes more than one protein or target. It is understood that, in certain embodiments, a binding agent that specifically binds a first target may or may not specifically bind a second target. As such, “specific binding” does not necessarily require (although it can include) exclusive binding, i.e. binding to a single target. Thus, a binding agent may, in certain embodiments, specifically bind more than one target. In certain embodiments, multiple targets may be bound by the same antigen-binding site on the binding agent. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. By way of non-limiting example, a bispecific antibody may comprise one antigen-binding site that recognizes an epitope on one protein and further comprise a second, different antigen-binding site that recognizes a different epitope on a second protein. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies, in certain embodiments, the polypeptides can occur as single chains or associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

“Conditions of high stringency” may be identified by those that: (1) employ low ionic strength and high temperature for washing, for example 15 mM sodium chloride/1.5 mM sodium citrate (1×SSC) with 0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes in 0.2×SSC containing 50% formamide at 55° C., followed by a high-stringency wash consisting of 0.1×SSC containing EDTA at 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and/or antibodies of the invention do not abrogate the binding of the polypeptide or antibody containing the amino acid sequence, to the antigen(s), i.e., the one or more IgCAM protein(s) to which the polypeptide or antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (noncancerous) or malignant (cancerous) including pre-cancerous lesions.

The term “metastasis” as used herein refers to the process by which a cancer spreads or transfers from the site of origin to other regions of the body with the development of a similar cancerous lesion at a new location. A “metastatic” or “metastasizing” cell is one that loses adhesive contacts with neighboring cells and migrates via the bloodstream or lymph from the primary site of disease to invade neighboring body structures.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into immunocompromised hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to a product or compound approved (or approvable) by a regulatory agency of the Federal or a state government or listed in the U.S. Pharmacopoeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one binding agent (e.g., an antibody) of the present disclosure, and which does not destroy the pharmacological activity thereof and is nontoxic when administered in doses sufficient to deliver a therapeutic effect.

The terms “effective amount” or “therapeutically effective amount” or “therapeutic effect” refer to an amount of a binding agent, an antibody, polypeptide, polynucleotide, small organic molecule, or other drug effective to “treat” a disease or disorder in a subject such as, a mammal. In the case of cancer, the therapeutically effective amount of a drug (e.g., an antibody) has a therapeutic effect and as such can reduce the number of cancer cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer, reduce morbidity and mortality; improve quality of life; or a combination of such effects. To the extent the agent, for example an antibody, prevents growth and/or kills existing cancer cells, it can be referred to as cytostatic and/or cytotoxic.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In some embodiments, a subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. AGENTS THAT MODULATE THE HIPPO PATHWAY

The present invention provides agents that modulate the Hippo pathway. The Hippo pathway is modulated by cell-cell contact, implying that there are cell surface receptors that direct and/or control Hippo pathway signaling events. The identity of these receptors in mammalian cells has not been elucidated. A central characteristic of epithelial cell biology is that epithelial cells exist in single-cell layers. As such, they have three distinct surfaces, an apical surface exposed to the lumen, a basolateral membrane that interacts with the basement membrane, and an “intercellular surface” forming the interaction region between adjacent cells. We hypothesized that putative Hippo pathway receptors would generally be restricted to this third surface, the intercellular surface, as this would be the likely region to enable direct cell-cell communication. Many proteins, including cell adhesion molecules (CAMs) are involved in interactions of cells with each other and with their microenvironment. Some of these proteins are known to reside within the intercellular membrane region, including cadherins which contribute to adherens junctions, connexins which contribute to gap junctions, and claudins and occludin which contribute to tight junctions. In addition to these proteins, other proteins are thought to reside in the apical junctional complex created by the tight junctions and adherens junctions.

Interestingly, many of these intercellular surface proteins have been identified as receptors for a variety of viruses. It is likely that viruses have evolved to exploit the localization of these receptors within the intercellular interface, which may allow for enhanced viral spreading between cells without exposure of the viruses to the lumen where they would be more vulnerable to attack and clearance by the immune system. Among these proteins are CAR (or CXAR) which is a receptor for coxsackie virus and adenovirus, PVR which is the poliovirus receptor, PVRL1 which is a receptor for herpes virus, PVRL4 which is a receptor for measles virus, and junction adhesion molecule (JAM1 or JAM-A) which is a receptor for reovirus. See, for example, Barton et al., 2001, Cell 104:441-451; Gonzalez-Mariscal et al., 2009, Front. Biosci., 14:731-768; Muhlebach et al., 2011, Nature 480:530-533.

Many of the intercellular surface receptors are members of the immunoglobulin superfamily of cell adhesion molecules (IgCAMs) and are generally related in both structure and function. Most of these proteins are type I transmembrane proteins, which typically consist of an extracellular domain (ECD) which contains one or more Ig-like domains, a single transmembrane domain, and a cytoplasmic tail. IgCAM members mediate interactions through their N-terminal Ig-like domains, which commonly bind other Ig-like domains on an opposing cell surface (homophilic adhesion), and also interact with integrins and carbohydrates (heterophilic adhesion) (Wong t al., 2012, Int. J. Cell Biol; epub). Interestingly, many of the IgCAMs molecules are also expressed on immune cells suggesting that immunosurveillance mechanisms might be triggered if their normal localization within the intercellular interface is altered. In seeking to identify receptors that modulate the Hippo pathway, we hypothesized that this particular subset of the immunoglobulin superfamily would be well-positioned to mediate intercellular signaling in the Hippo pathway.

Thus, in some embodiments, agents that modulate the Hippo pathway specifically bind the extracellular domain of a cell adhesion molecule of the immunoglobulin superfamily (IgCAM). These agents are referred to herein as “IgCAM-binding agents”. In some embodiments, the IgCAM-binding agents are antibodies. In some embodiments, the IgCAM-binding agents are soluble receptors. In some embodiments, the IgCAM-binding agents comprise soluble receptors. In some embodiments, the IgCAM-binding agents are polypeptides. In some embodiments, the IgCAM-binding agents are fusion proteins. In some embodiments, the IgCAM-binding agents are small molecules. In some embodiments, the IgCAM-binding agent are small peptides. In certain embodiments, the IgCAM-binding agent binds at least one IgCAM selected from the JAM family, the PVR family, and/or the CADM family. In certain embodiments, the IgCAM-binding agent binds at least one IgCAM selected from the JAM family. In certain embodiments, the IgCAM-binding agent binds at least one IgCAM selected from the PVR family. In certain embodiments, the IgCAM-binding agent binds at least one IgCAM selected from the CADM family. In certain embodiments, the IgCAM-binding agent binds at least one IgCAM selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In certain embodiments, the IgCAM-binding agent binds at least one IgCAM selected from the group consisting of: AMICA, CAR, CLMP, ESAM, GPA33, VSIG1, VSIG2, VSIG3, VSIG4, VSIG8, JAM1, JAM2, JAM3, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In some embodiments, the IgCAM-binding agent binds CLMP, VSIG4, VSIG8, JAM2, JAM3, CADM1, CADM3, CADM4, PVRL1, PVRL3, PVRL4, TIGIT, TMIGD1, and/or TMEM25. In some embodiments, the IgCAM-binding agent binds CADM1, CADM3, PVRL1 and/or PVRL3. In some embodiments, the IgCAM-binding agent binds CADM3, PVRL1 and/or PVRL3. In some embodiments, the IgCAM-binding agent binds CADM3. In some embodiments, the IgCAM-binding agent does not bind CADM1. In certain embodiments, the IgCAM-binding agent specifically binds more than one IgCAM. The full-length amino acid (aa) sequences for many human IgCAMs are known in the art and several are provided herein as SEQ ID NO: 1 (AMICA), SEQ ID NO:2 (CAR), SEQ ID NO:3 (CLMP), SEQ ID NO:4 (ESAM), SEQ ID NO:5 (GPA33), SEQ ID NO:6 (JAM1), SEQ ID NO:7 (JAM2), SEQ ID NO:8 (JAM3), SEQ ID NO:9 (VSIG1), SEQ ID NO:10 (VSIG2), SEQ ID NO:11 (VSIG3), SEQ ID NO:12 (VSIG4), SEQ ID NO:13 (VSIG8), SEQ ID NO:14 (CADM1), SEQ ID NO:15 (CADM2), SEQ ID NO:16 (CADM3), SEQ ID NO: 17 (CADM4), SEQ ID NO:18 (CRTAM), SEQ ID NO: 19 (TMIGD1), SEQ ID NO:20 (CD96), SEQ ID NO:21 (CD200), SEQ ID NO:22 (CD200R1), SEQ ID NO:23 (CD200R1L), SEQ ID NO:24 (CD226), SEQ ID NO:25 (PVRIG), SEQ ID NO:26 (PVR), SEQ ID NO:27 (PVRL1), SEQ ID NO:28 (PVRL2), SEQ ID NO:29 (PVRL3), SEQ ID NO:30 (PVRL4), SEQ ID NO:31 (TIGIT), and SEQ ID NO:32 (TMEM25).

In some embodiments, the IgCAM-binding agent is an agonist of at least one IgCAM. In certain embodiments, the IgCAM-binding agent is an agonist of at least one IgCAM selected from the JAM family, the PVR family, and/or the CADM family. In certain embodiments, the IgCAM-binding agent is an agonist of at least one IgCAM selected from the JAM family. In certain embodiments, the IgCAM-binding agent is an agonist of at least one IgCAM selected from the PVR family. In certain embodiments, the IgCAM-binding agent is an agonist of at least one IgCAM selected from the CADM family. In certain embodiments, the IgCAM-binding agent is an agonist of at least one IgCAM selected from the group consisting of AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In certain embodiments, the IgCAM-binding agent is an agonist of at least one IgCAM selected from the group consisting of: AMICA, CAR, CLMP, ESAM, GPA33, VSIG1, VSIG2, VSIG3, VSIG4, VSIG8, JAM1, JAM2, JAM3, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In some embodiments, the IgCAM-binding agent is an agonist of CLMP, VSIG4, VSIG8, JAM2, JAM3, CADM3, CADM4, PVRL1, PVRL3, PVRL4, TIGIT, TMIGD1, and/or TMEM25. In some embodiments, the IgCAM-binding agent is an agonist of CADM1, CADM3, PVRL1 and/or PVRL3. In some embodiments, the IgCAM-binding agent is an agonist of CADM3, PVRL1 and/or PVRL3. In some embodiments, the IgCAM-binding agent is an agonist of CADM3.

In certain embodiments, the IgCAM-binding agent is an agonist of the Hippo pathway. In some embodiments, the IgCAM-binding agent activates Hippo pathway signaling. In some embodiments, the IgCAM-binding agent induces Hippo pathway signaling. In some embodiments, the IgCAM-binding agent enhances Hippo pathway signaling. In some embodiments, the IgCAM-binding agent inhibits YAP activation. In some embodiments, the IgCAM-binding agent inhibits YAP translocation to the nucleus. In some embodiments, the IgCAM-binding agent enhances or increases YAP phosphorylation. In some embodiments, the IgCAM-binding agent enhances or increases YAP degradation. In some embodiments, the IgCAM-binding agent enhances or increases YAP retention or sequestration in the cell cytoplasm. In some embodiments, the IgCAM-binding agent reduces expression of YAP-dependent genes or proteins encoded by YAP-dependent genes. The term “YAP-dependent gene(s)” refers to genes whose expression is modulated (e.g., increased or decreased) by YAP. This term is used to reflect either the collective group of genes, subsets, or individual genes whose expression is modulated by the combination of YAP and a transcription factor. In some embodiments, the IgCAM-binding agent inhibits TAZ activation. In some embodiments, the IgCAM-binding agent inhibits TAZ translocation to the nucleus. In some embodiments, the IgCAM-binding agent enhances or increases TAZ phosphorylation. In some embodiments, the IgCAM-binding agent enhances or increases TAZ degradation. In some embodiments, the IgCAM-binding agent enhances or increases TAZ retention in the cell cytoplasm. In some embodiments, the IgCAM-binding agent reduces expression of TAZ-dependent genes or proteins encoded by TAZ-dependent genes. The term “TAZ-dependent genes)” refers to genes whose expression is modulated (e.g., increased or decreased) by TAZ. This term is used to reflect either the collective group of genes, subsets, or individual genes whose expression is modulated by the combination of TAZ and a transcription factor. In some embodiments, the IgCAM-binding agent reduces expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, AREG, FN1, ADAMTS1, ADAMTS5, AXL, MET, CYR61, IL8, ZEB1, FOXC2, N-cadherin, and SNAIL.

In certain embodiments, the IgCAM-binding agent enhances contact-dependent growth inhibition. In certain embodiments, the IgCAM-binding agent reduces cell proliferation. In certain embodiments, the IgCAM-binding agent inhibits anchorage-independent growth.

In some embodiments, the IgCAM-binding agent is an antagonist of at least one IgCAM. In certain embodiments, the IgCAM-binding agent is an antagonist of at least one IgCAM selected from the JAM family, the PVR family, and/or the CADM family. In certain embodiments, the IgCAM-binding agent is an antagonist of at least one IgCAM selected from the JAM family. In certain embodiments, the IgCAM-binding agent is an antagonist of at least one IgCAM selected from the PVR family. In certain embodiments, the IgCAM-binding agent is an antagonist of at least one IgCAM selected from the CADM family. In certain embodiments, the IgCAM-binding agent is an antagonist of at least one IgCAM selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In certain embodiments, the IgCAM-binding agent is an antagonist of at least one IgCAM selected from the group consisting of: AMICA, CAR, CLMP, ESAM, GPA33, VSIG1, VSIG2, VSIG3, VSIG4, VSIG8, JAM1, JAM2, JAM3, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In some embodiments, the IgCAM-binding agent is an antagonist of CLMP, VSIG4, VSIG8, JAM2, JAM3, CADM3, CADM4, PVRL1, PVRL3, PVRL4, TIGIT, TMIGD1, and/or TMEM25. In some embodiments, the IgCAM-binding agent is an antagonist of CADM1, CADM3, PVRL1 and/or PVRL3. In some embodiments, the IgCAM-binding agent is an antagonist of CADM3, PVRL1 and/or PVRL3. In some embodiments, the IgCAM-binding agent is an antagonist of CADM3.

In some embodiments, the IgCAM-binding agent is an antagonist of the Hippo pathway. In some embodiments, the IgCAM-binding agent decreases Hippo pathway signaling. In some embodiments, the IgCAM-binding agent inhibits Hippo pathway signaling. In some embodiments, the IgCAM-binding agent suppresses Hippo pathway signaling. In some embodiments, the IgCAM-binding agent increases YAP activation. In some embodiments, the IgCAM-binding agent promotes YAP translocation to the nucleus. In some embodiments, the IgCAM-binding agent suppresses or decreases YAP phosphorylation. In some embodiments, the IgCAM-binding agent suppresses or decreases YAP degradation. In some embodiments, the IgCAM-binding agent suppresses or decreases YAP retention in the cell cytoplasm. In some embodiments, the IgCAM-binding agent increases TAZ activation. In some embodiments, the IgCAM-binding agent promotes TAZ translocation to the nucleus. In some embodiments, the IgCAM-binding agent suppresses or decreases TAZ phosphorylation. In some embodiments, the IgCAM-binding agent suppresses or decreases TAZ degradation. In some embodiments, the IgCAM-binding agent suppresses or decreases TAZ retention in the cell cytoplasm. In some embodiments, the IgCAM-binding agent increases expression of genes suppressed by the Hippo signaling pathway. In some embodiments, the IgCAM-binding agent increases expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, AREG, FN1, ADAMTS1, AXL, ADAMTS5, MET, CYR61, IL8, ZEB1, FOXC2, N-cadherin, and SNAIL.

In some embodiments, the IgCAM-binding agent binds at least one IgCAM and interferes with the interaction of the IgCAM with a second protein. In some embodiments, the IgCAM-binding agent binds at least one IgCAM and interferes with the interaction of the IgCAM with a second IgCAM. In some embodiments, the IgCAM-binding agent is an antibody that interferes with the interaction of at least one IgCAM with at least one second IgCAM. In some embodiments, the IgCAM-binding agent comprises an antibody that interferes with the interaction of at least one IgCAM with at least one second IgCAM. In some embodiments, the IgCAM-binding agent is a soluble receptor that interferes with the interaction of at least one IgCAM with at least one second IgCAM. In some embodiments, the IgCAM-binding agent comprises a soluble receptor that interferes with the interaction of at least one IgCAM with at least one second IgCAM. In some embodiments, the IgCAM-binding agent is a small molecule that interferes with the interaction of at least one IgCAM with at least one second IgCAM. In some embodiments, the IgCAM-binding agent is a small peptide that interferes with the interaction of at least one IgCAM with at least one second IgCAM.

In some embodiments, the IgCAM-binding agent binds at least one IgCAM protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, an IgCAM-binding agent binds an IgCAM with a K_{D} of about 1 nM or less. In some embodiments, an IgCAM-binding agent binds an IgCAM with a K_{D} of about 0.1 nM or less. In certain embodiments, an IgCAM-binding agent described herein binds at least one additional IgCAM. In some embodiments, an IgCAM-binding agent binds a human IgCAM with a K_{D} of about 0.1 nM or less. In some embodiments, the IgCAM-binding agent binds both a human IgCAM and a mouse IgCAM with a K_{D} of about 10 nM or less. In some embodiments, an IgCAM-binding agent binds both a human IgCAM and a mouse IgCAM with a K_{D} of about 1 nM or less. In some embodiments, an IgCAM-binding agent binds both a human IgCAM and a mouse IgCAM with a K_{D} of about 0.11 nM or less. In some embodiments, the dissociation constant of the binding agent to an IgCAM is the dissociation constant determined using an IgCAM fusion protein comprising at least a portion of the IgCAM immobilized on a Biacore chip.

In some embodiments, the IgCAM-binding agent binds a human IgCAM with a half maximal effective concentration (EC₅₀) of about 1 μM or less, about 100 nM or less, about 40 nM or less, about 20 nM or less, about 10 nM or less, about 1 nM or less, or about 0.1 nM or less. In certain embodiments, an IgCAM-binding agent also binds at least one additional IgCAM with an EC₅₀ of about 40 nM or less, about 20 nM or less, about 10 nM or less, about 1 nM or less or about 0.1 nM or less.

In some embodiments, the IgCAM-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent. In some embodiments, the antibody is bivalent. In some embodiments, the antibody is monospecific. In some embodiments, the antibody is bispecific or multispecific. In some embodiments, the antibody is conjugated to a cytotoxic moiety. In some embodiments, the antibody is isolated. In some embodiments, the antibody is substantially pure.

In some embodiments, the IgCAM-binding agents are polyclonal antibodies. Polyclonal antibodies can be prepared by any known method. In some embodiments, polyclonal antibodies are raised by immunizing an animal (e.g., a rabbit, rat, mouse, goat, or donkey) by multiple subcutaneous or intraperitoneal injections of the relevant antigen (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, ascites, and the like, of the immunized animal. The polyclonal antibodies can be purified from serum or ascites according to standard methods in the art including, but not limited to, affinity chromatography, ion-exchange chromatography, gel electrophoresis, and dialysis.

In some embodiments, the IgCAM-binding agents are monoclonal antibodies. Monoclonal antibodies can be prepared using hybridoma methods known to one of skill in the art (see e.g., Kohler and Milstein, 1975, Nature, 256:495-497). In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods (J. W. Goding, 1996, Monoclonal Antibodies: Principles and Practice, 3^{rd} Edition, Academic Press, San Diego, Calif.) or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art (see e.g., U.S. Pat. No. 4,816,567). The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries expressing CDRs and/or variable regions of the desired species (see e.g., McCafferty et al., 1990, Nature, 348:552-554; Clackson et al., 1991, Nature, 352:624-628; and Marks t al., 1991, J. Mol. Biol., 222:581-597).

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region(s) can be used to optimize specificity, affinity, etc. of a monoclonal antibody.

In some embodiments, the monoclonal antibody against a human IgCAM is a humanized antibody. Typically, humanized antibodies are human immunoglobulins in which residues from the CDRs are replaced by residues from a CDR of a non-human species (e.g., mouse, rat, rabbit, hamster, etc.) that have the desired specificity, affinity, and/or binding capability using methods known to one skilled in the art. In some embodiments, the Fv framework region residues of a human immunoglobulin are replaced with the corresponding residues in an antibody from a non-human species that has the desired specificity, affinity, and/or binding capability. In some embodiments, the humanized antibody can be further modified by the substitution of additional residues either in the Fv framework region and/or within the replaced non-human residues to refine and optimize antibody specificity, affinity, and/or capability. The humanized antibody may comprise substantially all of at least one, and typically two or three, variable domain regions containing all, or substantially all, of the CDRs that correspond to the non-human immunoglobulin whereas all, or substantially all, of the framework regions are those of a human immunoglobulin consensus sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques (see e.g., U.S. Pat. Nos. 5,225,539; 5,585,089; 5,693,761; and 5,693,762).

In some embodiments, the IgCAM-binding agent is a human antibody. Human antibodies can be directly prepared using various techniques known in the art. In some embodiments, immortalized human B lymphocytes immunized in vitro or isolated from an immunized individual that produces an antibody directed against a target antigen can be generated (see, e.g., Cole et al., 1985, Monoclonal Antibodies and Cancer Therapy, Alan R. Liss, p. 77; Boerner et al., 1991, J. Immunol., 147:86-95; and U.S. Pat. Nos. 5,750,373; 5,567,610 and 5,229,275). In some embodiments, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Mol. Biol., 227:381; Marks et al., 1991, J. Mol. Biol., 222:581). Alternatively, phage display technology can be used to produce human antibodies and antibody fragments in vitro, from immunoglobulin variable domain gene repertoires from unimmunized donors. Techniques for the generation and use of antibody phage libraries are also described in U.S. Pat. Nos. 5,969,108; 6,172,197; 5,885,793; 6,521,404; 6,544,731; 6,555,313; 6,582,915; 6,593,081; 6,300,064; 6,653,068; 6,706,484; and 7,264,963; and Rothe et al., 2008, J. Mol. Bio., 376:1182-1200. Affinity maturation strategies including, but not limited to, chain shuffling (Marks et al., 1992, Bio/Technology, 10:779-783) and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

This invention also encompasses bispecific antibodies that specifically recognize at least one human IgCAM protein. Bispecific antibodies are capable of specifically recognizing and binding at least two different epitopes. The different epitopes can either be within the same molecule (e.g., two epitopes on one human IgCAM) or on different molecules (e.g., one epitope on a human IgCAM and one epitope on a second molecule). In some embodiments, the bispecific antibodies are monoclonal human or humanized antibodies. In some embodiments, the antibodies can specifically recognize and bind a first antigen target, (e.g., an IgCAM) as well as a second antigen target, such as an effector molecule on a leukocyte (e.g., CD2, CD3, CD28, CD80, or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA.

Techniques for making bispecific antibodies are known by those skilled in the art, see for example, Millstein et al., 1983, Nature, 305:537-539; Brennan et al., 1985, Science, 229:81; Suresh et al., 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; U.S. Pat. No. 5,731,168; and U.S. Patent Publication No. 2011/0123532. Bispecific antibodies can be intact antibodies or antibody fragments. Antibodies with more than two valencies are also contemplated. For example, trispecific antibodies can be prepared (Tutt et al., 1991, J. Immunol., 147:60). Thus, in certain embodiments the antibodies to IgCAMs are multispecific.

In certain embodiments, the antibodies (or other polypeptides) described herein may be monospecific. For example, in certain embodiments, each of the one or more antigen-binding sites that an antibody contains is capable of binding (or binds) a homologous epitope on more than one IgCAM. In certain embodiments, an antigen-binding site of a monospecific antibody described herein is capable of binding (or binds), for example, PVR and PVRL1 (i.e., the same epitope is found on both PVR and PVRL1 proteins).

In certain embodiments, the IgCAM-binding agent is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries (Huse et al., 1989, Science, 246:1275-1281) to allow rapid and effective identification of monoclonal Fab fragments with the desired specificity for an IgCAM or derivatives, fragments, analogs or homologs thereof. In some embodiments, antibody fragments are linear antibody fragments. In certain embodiments, antibody fragments are monospecific or bispecific. In certain embodiments, the IgCAM-binding agent is a scFv. Various techniques can be used for the production of single-chain antibodies specific to one or more human IgCAMs and are known to those of skill in the art.

It can further be desirable, especially in the case of antibody fragments, to modify an antibody in order to increase (or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., a human IgCAM). In this regard, the variable region may comprise or be derived from any type of mammal that can be induced to mount a humoral response and generate immunoglobulins against the desired tumor associated antigen. As such, the variable region of the modified antibodies can be, for example, of human, murine, non-human primate (e.g. cynomolgus monkeys, macaques, etc.), or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived from an antibody of different class and preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain. In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that controls a specific effector function (e.g. complement C1q binding) to be modulated. Such partial deletions of the constant regions may improve selected characteristics of the antibody (e.g., serum half-life) while leaving other desirable functions associated with the constant region intact. Moreover, as alluded to above, the constant regions of the disclosed antibodies may be modified through the mutation or substitution of one or more amino acids that enhances the function of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (when the antibodies are bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the IgCAM-binding antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using biochemical or molecular engineering techniques well-known to the skilled artisan.

In certain embodiments, an IgCAM-binding agent that is an antibody does not have one or more effector functions. For instance, in some embodiments, the antibody has no ADCC activity, and/or no complement-dependent cytotoxicity (CDC) activity. In certain embodiments, the antibody does not bind an Fc receptor and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art and described herein.

Thus, the present invention provides methods for producing an antibody that binds at least one IgCAM. In some embodiments, the method for producing an antibody that binds at least one IgCAM comprises using hybridoma techniques. In some embodiments, a method for producing an antibody that binds the extracellular domain of a human IgCAM is provided. In some embodiments, a method for producing an antibody that binds the extracellular domain of a human IgCAM selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25 is provided. In some embodiments, a method for producing an antibody that binds the extracellular domain of a human IgCAM selected from the group consisting of: AMICA, CAR, CLMP, ESAM, GPA33, VSIG1, VSIG2, VSIG3, VSIG4, VSIG8, JAM1, JAM2, JAM3, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25 is provided. In some embodiments, the method comprises using SEQ ID NO:33 or a portion thereof as an immunogen. In some embodiments, the method comprises using SEQ ID NO:34 or a portion thereof as an immunogen. In some embodiments, the method comprises using SEQ ID NO:35 or a portion thereof as an immunogen. In some embodiments, the method comprises using SEQ ID NO:36 or a portion thereof as an immunogen. In some embodiments, the method comprises using SEQ ID NO:37 or a portion thereof as an immunogen. In some embodiments, the method comprises using SEQ ID NO:38 or a portion thereof as an immunogen. In some embodiments, the method comprises using SEQ ID NO:39 or a portion thereof as an immunogen. In some embodiments, the method comprises using SEQ ID NO:40 or a portion thereof as an immunogen. In some embodiments, the method comprises using SEQ ID NO:41 or a portion thereof as an immunogen. In some embodiments, the method comprises using SEQ ID NO:42 or a portion thereof as an immunogen. In some embodiments, the method comprises using SEQ ID NO:43 or a portion thereof as an immunogen. In some embodiments, the method comprises using SEQ ID NO:44 or a portion thereof as an immunogen. In some embodiments, the method comprises using SEQ ID NO:45 or a portion thereof as an immunogen. In some embodiments, the method comprises using SEQ ID NO:46 or a portion thereof as an immunogen. In some embodiments, the method comprises using SEQ ID NO:47 or a portion thereof as an immunogen. In some embodiments, the method comprises using SEQ ID NO:48 or a portion thereof as an immunogen. In some embodiments, the method comprises using SEQ ID NO:49 or a portion thereof as an immunogen. In some embodiments, the method comprises using SEQ ID NO:50 or a portion thereof as an immunogen. In some embodiments, the method comprises using SEQ ID NO:51 or a portion thereof as an immunogen. In some embodiments, the method comprises using SEQ ID NO:52 or a portion thereof as an immunogen. In some embodiments, the method comprises using SEQ ID NO:53 or a portion thereof as an immunogen. In some embodiments, the method comprises using SEQ ID NO:54 or a portion thereof as an immunogen. In some embodiments, the method comprises using SEQ ID NO:55 or a portion thereof as an immunogen. In some embodiments, the method comprises using SEQ ID NO:56 or a portion thereof as an immunogen. In some embodiments, the method comprises using SEQ ID NO:57 or a portion thereof as an immunogen. In some embodiments, the method comprises using SEQ ID NO:58 or a portion thereof as an immunogen. In some embodiments, the method comprises using SEQ ID NO:59 or a portion thereof as an immunogen. In some embodiments, the method comprises using SEQ ID NO:60 or a portion thereof as an immunogen. In some embodiments, the method comprises using SEQ ID NO:61 or a portion thereof as an immunogen. In some embodiments, the method comprises using SEQ ID NO:62 or a portion thereof as an immunogen. In some embodiments, the method comprises using SEQ ID NO:63 or a portion thereof as an immunogen. In some embodiments, the method comprises using SEQ ID NO:64 or a portion thereof as an immunogen.

In some embodiments, the method of generating an antibody that binds at least one human IgCAM comprises screening a human phage library. The present invention further provides methods of identifying an antibody that binds at least one IgCAM. In some embodiments, the antibody is identified by screening using FACS for binding to an IgCAM or a portion thereof. In some embodiments, the antibody is identified by screening using ELISA for binding to an IgCAM or a portion thereof. In some embodiments, the antibody is identified by screening for the effect on cell morphology in a clonogenic assay. In some embodiments, the antibody is identified by screening for the effect on cell growth and/or proliferation in a clonogenic assay. In some embodiments, the antibody is identified by screening for activation or enhancement of Hippo pathway signaling. In some embodiments, the antibody is identified by screening for inhibition of YAP activation. In some embodiments, the antibody is identified by screening for translocation of YAP. In some embodiments, the antibody is identified by screening for YAP phosphorylation. In some embodiments, the antibody is identified by screening for inhibition of TAZ activation. In some embodiments, the antibody is identified by screening for translocation of TAZ. In some embodiments, the antibody is identified by screening for TAZ phosphorylation.

In some embodiments, a method of generating an antibody to a human IgCAM comprises immunizing a mammal with a polypeptide comprising the extracellular domain of a human IgCAM. In some embodiments, a method of generating an antibody to a human IgCAM comprises immunizing a mammal with a polypeptide comprising at least a portion of the extracellular domain from AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, or TMEM25. In some embodiments, a method of generating an antibody to a human IgCAM comprises immunizing a mammal with a polypeptide comprising at least a portion of SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64. In some embodiments, the method further comprises isolating antibodies or antibody-producing cells from the mammal.

In some embodiments, a method of generating a monoclonal antibody which binds a human IgCAM comprises: (a) immunizing a mammal with a polypeptide comprising at least a portion of the extracellular domain from AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, or TMEM25; (b) isolating antibody-producing cells from the immunized mammal; (c) fusing the antibody-producing cells with cells of a myeloma cell line to form hybridoma cells. In some embodiments, the method further comprises (d) selecting a hybridoma cell expressing an antibody that binds at least one IgCAM.

In some embodiments, the antibody generated by the methods described herein is a Hippo pathway agonist. In some embodiments, the antibody generated by the methods described herein activates, increases, and/or enhances Hippo pathway signaling. In some embodiments, the antibody generated by the methods described herein increases YAP phosphorylation. In some embodiments, the antibody generated by the methods described herein increases YAP degradation. In some embodiments, the antibody generated by the methods described herein increases retention of YAP in the cytoplasm. In some embodiments, the antibody generated by the methods described herein inhibits YAP activation. In some embodiments, the antibody generated by the methods described herein inhibits YAP translocation to the nucleus. In some embodiments, the antibody generated by the methods described herein increases TAZ phosphorylation. In some embodiments, the antibody generated by the methods described herein increases TAZ degradation. In some embodiments, the antibody generated by the methods described herein increases retention of TAZ in the cytoplasm. In some embodiments, the antibody generated by the methods described herein inhibits TAZ activation. In some embodiments, the antibody generated by the methods described herein inhibits TAZ translocation to the nucleus.

In some embodiments, the antibody generated by the methods described herein is a Hippo pathway antagonist. In some embodiments, the antibody generated by the methods described herein inhibits, decreases, and/or suppresses Hippo pathway signaling. In some embodiments, the antibody generated by the methods described herein decreases YAP phosphorylation. In some embodiments, the antibody generated by the methods described herein decreases YAP degradation. In some embodiments, the antibody generated by the methods described herein decreases retention of YAP in the cytoplasm. In some embodiments, the antibody generated by the methods described herein increases or enhances YAP activation. In some embodiments, the antibody generated by the methods described herein increases or enhances YAP translocation to the nucleus. In some embodiments, the antibody generated by the methods described herein decreases TAZ phosphorylation. In some embodiments, the antibody generated by the methods described herein decreases TAZ degradation. In some embodiments, the antibody generated by the methods described herein decreases retention of TAZ in the cytoplasm. In some embodiments, the antibody generated by the methods described herein increases or enhances TAZ activation. In some embodiments, the antibody generated by the methods described herein increases or enhances TAZ translocation to the nucleus.

In some embodiments, a method of producing an antibody to at least one human IgCAM comprises identifying an antibody using a membrane-bound heterodimeric molecule comprising a single antigen-binding site. In some non-limiting embodiments, the antibody is identified using methods and polypeptides described in International Publication WO 2011/100566, which is incorporated by reference herein in its entirety.

In some embodiments, a method of producing an antibody to at least one human IgCAM comprises screening an antibody-expressing library for antibodies that bind at least one human IgCAM. In some embodiments, the antibody-expressing library is a phage library. In some embodiments, the antibody-expressing library is a mammalian cell display library. In some embodiments, the screening comprises panning. In some embodiments, the antibody-expressing library is screened using at least a portion of the extracellular domain of a human IgCAM. In some embodiments, the antibody-expressing library is screened using at least a portion of the extracellular domain of a human IgCAM selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In some embodiments, antibodies identified in the first screening, are screened again using a different IgCAM thereby identifying an antibody that binds more than one IgCAM.

In certain embodiments, the antibodies described herein are isolated. In certain embodiments, the antibodies described herein are substantially pure.

In certain embodiments, the IgCAM-binding agent is a soluble receptor. In certain embodiments, the IgCAM-binding agent comprises the extracellular domain of an IgCAM. In some embodiments, the IgCAM-binding agent comprises a fragment of the extracellular domain of an IgCAM (e.g., the N-terminal domain of an IgCAM). In some embodiments, soluble receptors comprising a fragment of the extracellular domain of an IgCAM can demonstrate altered biological activity (e.g., increased protein half-life) compared to soluble receptors comprising the entire IgCAM ECD. Protein half-life can be further increased by covalent modification with polyethylene glycol (PEG) or polyethylene oxide (PEO). In certain embodiments, the IgCAM is a human IgCAM. In certain embodiments, the IgCAM ECD or a fragment of the IgCAM ECD is a human IgCAM ECD selected from the JAM family, the PVR family, or the CADM family. In certain embodiments, the IgCAM ECD is a human IgCAM ECD selected from the JAM family. In certain embodiments, the IgCAM ECD is a human IgCAM ECD selected from the PVR family. In certain embodiments, the IgCAM ECD is a human IgCAM ECD selected from the CADM family. In certain embodiments, the human IgCAM ECD is an IgCAM ECD from AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, or TMEM25. In certain embodiments, the human IgCAM ECD is an IgCAM ECD from AMICA, CAR, CLMP, ESAM, GPA33, VSIG1, VSIG2, VSIG3, VSIG4, VSIG8, JAM1, JAM2, JAM3, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, or TMEM25. In some embodiments, the human IgCAM ECD is an IgCAM ECD from CLMP, VSIG4, VSIG8, JAM2, JAM3, CADM3, CADM4, PVRL1, PVRL3, PVRL4, TIGIT, TMIGD1, and/or TMEM25. In some embodiments, the human IgCAM ECD is an IgCAM ECD from CADM1, CADM3, PVRL1 and/or PVRL3. In some embodiments, the human IgCAM ECD is an IgCAM ECD from CADM3, PVRL1 and/or PVRL3. In some embodiments the human IgCAM ECD is an IgCAM ECD from CADM3. In some embodiments, the human IgCAM ECD is not from CADM1.

The predicted ECD domains for AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25 are provided as SEQ ID NOs:33-64. Those of skill in the art may differ in their understanding of the exact amino acids corresponding to the various ECD domains. Thus, the N-terminus and/or C-terminus of the ECDs described herein may extend or be shortened by 1, 2, 3, 4, 5, 6, 7, 8, 9, 10 or more amino acids.

In some embodiments, the IgCAM-binding agent comprises a sequence selected from the group consisting of: SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, and SEQ ID NO:64. In some embodiments, the IgCAM-binding agent comprises a fragment of a sequence selected from the group consisting of: SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45. SEQ ID NO:46, SEQ ID NO:47, SEQ ID NO:48, SEQ ID NO:49, SEQ ID NO:50, SEQ ID NO:51. SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, and SEQ ID NO:64.

In certain embodiments, the IgCAM-binding agent comprises a variant of any one of the aforementioned IgCAM ECD sequences that comprises one or more (e.g., one, two, three, four, five, six, seven, eight, nine, ten, etc.) conservative substitutions and is capable of binding IgCAM(s).

In some embodiments, the IgCAM-binding agent, such as a soluble receptor comprising the ECD of a human IgCAM, is a fusion protein. As used herein, a “fusion protein” is a hybrid protein expressed by a nucleic acid molecule comprising nucleotide sequences of at least two genes. In certain embodiments, the IgCAM-binding agent, such as a soluble receptor comprising the ECD of a human IgCAM, further comprises a non-IgCAM polypeptide. In some embodiments, IgCAM soluble receptors may include an IgCAM ECD linked to other non-IgCAM functional and structural polypeptides including, but not limited to, a human Fc region, protein tags (e.g., myc, FLAG, GST), other endogenous proteins or protein fragments, or any other useful protein sequence including any linker region between an IgCAM ECD and a second polypeptide. In certain embodiments, the non-IgCAM polypeptide is a human Fc region. The Fc region can be obtained from any of the classes of immunoglobulin, IgG, IgA, IgM, IgD and IgE. In some embodiments, the Fc region is a human IgG1 Fc region. In some embodiments, the Fc region is a human IgG2 Fc region. In some embodiments, the Fc region is a wild-type Fc region. In some embodiments, the Fc region is a natural variant of a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, the Fc region is truncated at the C-terminal end (e.g., lysine is absent). In some embodiments, an amino acid in the hinge domain is changed to hinder undesirable disulfide bond formation. In some embodiments, a cysteine is replaced with a different amino acid to hinder undesirable disulfide bond formation. In some embodiments, a cysteine is replaced with a serine to hinder undesirable disulfide bond formation. In certain embodiments, the non-IgCAM polypeptide comprises SEQ ID NO:65, SEQ ID NO:66, or SEQ ID NO:67. In certain embodiments, the non-IgCAM polypeptide consists essentially of SEQ ID NO:65, SEQ ID NO:66, or SEQ ID NO:67.

In certain embodiments, an IgCAM-binding agent is a fusion protein comprising at least a portion of an IgCAM ECD and a Fc region. In some embodiments, the C-terminus of the IgCAM ECD is linked to the N-terminus of the immunoglobulin Fc region. In some embodiments, the IgCAM ECD is directly linked to the Fc region (i.e. without an intervening peptide linker). In some embodiments, the IgCAM ECD is linked to the Fc region via a peptide linker.

As used herein, the term “linker” refers to a linker inserted between a first polypeptide (e.g., a IgCAM ECD or portion thereof) and a second polypeptide (e.g., a Fc region). In some embodiments, the linker is a peptide linker. Linkers should not adversely affect the expression, secretion, or bioactivity of the fusion protein. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. Linkers may include, but are not limited to, SerGly, GGSG, GSGS, GGGS, S(GGS)n where n is 1-7, GRA, poly(Gly), poly(Ala), ESGGGGVT (SEQ ID NO:74), LESGGGGVT (SEQ ID NO:75), GRAQVT (SEQ ID NO:76), WRAQVT (SEQ ID NO:77), and ARGRAQVT (SEQ ID NO:78). As used herein, a linker is an intervening peptide sequence that does not include amino acid residues from either the C-terminus of the first polypeptide (e.g., an IgCAM ECD) or the N-terminus of the second polypeptide (e.g., the Fc region).

In some embodiments, the IgCAM-binding agent comprises a first polypeptide comprising SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64; and a second polypeptide comprising SEQ ID NO:65, SEQ ID NO:66, or SEQ ID NO:67, wherein the first polypeptide is directly linked to the second polypeptide.

In some embodiments, the IgCAM-binding agent comprises a first polypeptide comprising SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64; and a second polypeptide comprising SEQ ID NO:65, SEQ ID NO:66, or SEQ ID NO:67, wherein the first polypeptide is connected to the second polypeptide by a linker.

In some embodiments, the IgCAM-binding agent comprises a first polypeptide comprising a portion of SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64; and a second polypeptide comprising SEQ ID NO:65, SEQ ID NO:66, or SEQ ID NO:67, wherein the first polypeptide is directly linked to the second polypeptide.

In some embodiments, the IgCAM-binding agent comprises a first polypeptide comprising a portion of SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64; and a second polypeptide comprising SEQ ID NO:65, SEQ ID NO:66, or SEQ ID NO:67, wherein the first polypeptide is connected to the second polypeptide by a linker.

In some embodiments, the IgCAM-binding agent comprises a first polypeptide that is at least 80% identical to SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64; and a second polypeptide comprising SEQ ID NO:65, SEQ ID NO:66, or SEQ ID NO:67, wherein the first polypeptide is directly linked to the second polypeptide. In some embodiments, the first polypeptide is at least 85%, at least 90%, or at least 95% identical to SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64.

In some embodiments, the IgCAM-binding agent comprises a first polypeptide that is at least 80% identical to SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64; and a second polypeptide comprising SEQ ID NO:65, SEQ ID NO:66, or SEQ ID NO:67, wherein the first polypeptide is connected to the second polypeptide by a linker. In some embodiments, the first polypeptide is at least 85%, at least 90%, or at least 95% identical to SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, or SEQ ID NO:64.

IgCAMs generally contain a signal sequence that directs the transport of the proteins. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus. In some embodiments, the signal sequence of the polypeptide is not a native IgCAM signal sequence.

In certain embodiments, an IgCAM-binding agent comprises a Fc region of an immunoglobulin. Those skilled in the art will appreciate that some of the binding agents of this invention will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region may include additions, deletions, or substitutions of one or more amino acids in one or more domains. The modified fusion proteins disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 as residues) that provides some of the molecular flexibility typically imparted by the absent constant region domain.

In some embodiments, the modified fusion proteins are engineered to link the CH3 domain directly to the hinge region or to the first polypeptide. In other embodiments, a peptide spacer is inserted between the hinge region or the first polypeptide and the modified CH2 and/or CH3 domains. For example, constructs may be expressed wherein the CH2 domain has been deleted and the remaining CH3 domain (modified or unmodified) is joined to the hinge region or first polypeptide with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement C1q binding). Such partial deletions of the constant regions may improve selected characteristics of the binding agent (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors).

In some embodiments, the modified fusion proteins provide for altered effector functions that, in turn, affect the biological profile of the administered agent. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moiety attachment sites.

In certain embodiments, a modified fusion protein does not have one or more effector functions normally associated with an Fc region. In some embodiments, the agent has no ADCC activity, and/or no CDC activity. In certain embodiments, the agent does not bind to the Fc receptor and/or complement factors. In certain embodiments, the agent has no effector function.

The IgCAM-binding agents (e.g., antibodies or soluble receptors) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

For example, the specific binding of an IgCAM-binding agent (e.g., an antibody or a soluble receptor) to a human IgCAM such as PVR may be determined using ELISA. An ELISA assay comprises preparing antigen, coating wells of a 96 well microtiter plate with antigen, adding the IgCAM-binding agent conjugated to a detectable compound such as an enzymatic substrate (e.g. horseradish peroxidase or alkaline phosphatase) to the well, incubating for a period of time and detecting the presence of the antibody bound to the antigen. In some embodiments, the IgCAM-binding agent is not conjugated to a detectable compound, but instead a second conjugated antibody that recognizes the IgCAM-binding agent is added to the well. In some embodiments, instead of coating the well with the antigen, the IgCAM-binding agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art.

In another example, the specific binding of an IgCAM-binding agent (e.g., an antibody or a soluble receptor) to a human IgCAM may be determined using FACS. A FACS screening assay may comprise generating a cDNA construct that expresses an antigen as a fusion protein (e.g., PVR-CD4TM), transfecting the construct into cells, expressing the antigen on the surface of the cells, mixing the IgCAM-binding agent with the transfected cells, and incubating for a period of time. The cells bound by the IgCAM-binding agent may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an IgCAM-binding agent (e.g., an antibody or a soluble receptor) to an antigen/target (e.g., an IgCAM) and the off-rate of a binding agent-antigen/target interaction can be determined by competitive binding assays. One example of a competitive binding assay is a radioimmunoassay comprising the incubation of labeled antigen/target (e.g., ³H or ¹²⁵I), or fragment or variant thereof, with the binding agent of interest in the presence of increasing amounts of unlabeled antigen/target followed by the detection of the binding agent bound to the labeled antigen/target. The affinity of the binding agent for an antigen/target (e.g., an IgCAM) and the binding off-rates can be determined from the data by Scatchard plot analysis. In some embodiments, Biacore kinetic analysis is used to determine the binding on and off rates of binding agents that bind an antigen/target (e.g., an IgCAM). Biacore kinetic analysis comprises analyzing the binding and dissociation of binding agents from chips with immobilized antigen/target (e.g., an IgCAM) on the chip surface.

In certain embodiments, the IgCAM-binding agent (e.g., an antibody or a soluble receptor) described herein binds at least one human IgCAM and modulates Hippo pathway activity. In some embodiments, the IgCAM-binding agent is an agonist of the Hippo pathway and activates and/or increases Hippo pathway signaling. In some embodiments, the IgCAM-binding agent is an agonist of the Hippo pathway and increases YAP phosphorylation. In some embodiments, the IgCAM-binding agent is an agonist of the Hippo pathway and increases YAP degradation. In some embodiments, the IgCAM-binding agent is an agonist of the Hippo pathway and reduces expression of YAP-dependent genes and/or proteins encoded by YAP-dependent genes. In some embodiments, the expression of the YAP-dependent gene is modulated by the combination of YAP and a transcription factor selected from the group consisting of: TEAD1, TEAD2, TEAD3, TEAD4, Smad1, Smad4, RUNX, ErbB4, and p73. As described herein, examples of YAP-dependent genes include, but are not limited to, CD44, CD47, CD133, TDGF1, EPHB2, CA12, LRP4, GPC4, CLDN2, CTGF, PAG1, SEMA4D, RHEB, MAGI1, ITPR3, CD168, NRP2, Gli2, BIRC2, BIRC5, FGF1, IL33, GRB2, IGFBP3, and AREG. In some embodiments, the IgCAM-binding agent is an agonist of the Hippo pathway and increases TAZ phosphorylation. In some embodiments, the IgCAM-binding agent is an agonist of the Hippo pathway and increases TAZ degradation. In some embodiments, the IgCAM-binding agent is an agonist of the Hippo pathway and reduces expression of TAZ-dependent genes. In some embodiments, the expression of the TAZ-dependent gene is modulated by the combination of TAZ and a transcription factor selected from the group consisting of: TEAD1, TEAD2, TEAD3, TEAD4, RUNX, ERBB4, SMAD2/3/4, PAX3, TBX5, TTF-1, PPARγ, and p73. Examples of TAZ-dependent genes include, but are not limited to, CTGF, CD44, FN1, BIRC5 (survivin), ADAMTS1, GLI2, AXL, ADAMTS5, MET, CYR61, IL8, ZEB1, FOXC2, N-cadherin, and SNAIL. In some embodiments, the IgCAM-binding agent is an agonist of the Hippo pathway and modulates (e.g., increases or decreases) expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, AREG, FN1, ADAMTS1, AXL, ADAMTS5, MET, CYR61, IL8, ZEB1, FOXC2, N-cadherin, and SNAIL.

In certain embodiments, the IgCAM-binding agent (e.g., an antibody or a soluble receptor) is an agonist of at least one human IgCAM. In some embodiments, the IgCAM-binding agent is an agonist of at least one IgCAM and activates and/or increases Hippo pathway activity. In certain embodiments, the IgCAM-binding agent increases Hippo pathway activity by at least about 10%, at least about 20%, at least about 30%, at least about 50%, at least about 75%, at least about 90%, or about 100%. In some embodiments, the IgCAM-binding agent increases activity of one, two, three, four, or more IgCAMs. In some embodiments, the IgCAM-binding agent increases activity of at least one of AMICA, CAR, CLMP, ESAM, GPA33, VSIG1, VSIG2, VSIG3, VSIG4, VSIG5, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, or TMEM25.

In certain embodiments, the IgCAM-binding agent (e.g., an antibody or a soluble receptor) inhibits cell growth or cell proliferation. In certain embodiments, the IgCAM-binding agent inhibits cell growth or cell proliferation by at least about 10%, at least about 20%, at least about 30%, at least about 50%, at least about 75%, at least about 90%, or about 100%. In certain embodiments, the IgCAM-binding agent (e.g., an antibody or a soluble receptor) affects cell growth or cell morphology in a clonogenic assay. In some embodiments, the IgCAM-binding agent is selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, or TMEM25.

In certain embodiments, the IgCAM-binding agent is an antagonist of the Hippo pathway. In some embodiments, the IgCAM-binding agent decreases Hippo pathway signaling. In some embodiments, the IgCAM-binding inhibits Hippo pathway signaling. In some embodiments, the IgCAM-binding agent suppresses Hippo pathway signaling. In some embodiments, the IgCAM-binding agent increases YAP activation. In some embodiments, the IgCAM-binding agent increases YAP translocation to the nucleus. In some embodiments, the IgCAM-binding agent suppresses or decreases YAP phosphorylation. In some embodiments, the IgCAM-binding agent suppresses or decreases YAP degradation. In some embodiments, the IgCAM-binding agent suppresses or decreases YAP retention in the cell cytoplasm. In some embodiments, the IgCAM-binding agent increases TAZ activation. In some embodiments, the IgCAM-binding agent increases TAZ translocation to the nucleus. In some embodiments, the IgCAM-binding agent suppresses or decreases TAZ phosphorylation. In some embodiments, the IgCAM-binding agent suppresses or decreases TAZ degradation. In some embodiments, the IgCAM-binding agent suppresses or decreases TAZ retention in the cell cytoplasm. In some embodiments, the IgCAM-binding agent reduces expression of genes in the Hippo signaling pathway. In some embodiments, the IgCAM-binding agent is an antagonist of the Hippo pathway and modulates (e.g., increases or decreases) expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, AREG, FN1, ADAMTS1, AXL, ADAMTS5, MET, CYR61, IL8, ZEB1, FOXC2, N-cadherin, and SNAIL.

In certain embodiments, the IgCAM-binding agent (e.g., an antibody or a soluble receptor) is an antagonist of at least one human IgCAM. In some embodiments, the IgCAM-binding agent is an antagonist of at least one IgCAM and suppresses and/or inhibits Hippo pathway activity. In certain embodiments, the IgCAM-binding agent decreases Hippo pathway activity by at least about 10%, at least about 20%, at least about 300/, at least about 50%, at least about 75%, at least about 90%, or about 100%. In some embodiments, the IgCAM-binding agent decreases activity of one, two, three, four, or more IgCAMs. In some embodiments, the IgCAM-binding agent decreases activity of at least one of AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, or TMEM25.

In certain embodiments, the IgCAM-binding agent (e.g., an antibody or a soluble receptor) increases cell growth or cell proliferation. In certain embodiments, the IgCAM-binding agent increases cell growth or cell proliferation by at least about 10%, at least about 20%, at least about 30%, at least about 50%, at least about 75%, at least about 90%, or about 100%. In some embodiments, the IgCAM-binding agent is selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, or TMEM25.

In certain embodiments, the IgCAM-binding agent (e.g., an antibody or a soluble receptor) is an antagonist of YAP activity. In some embodiments, the IgCAM-binding agent is an antagonist of YAP activity by increasing phosphorylation of YAP. In some embodiments, the IgCAM-binding agent is an antagonist of YAP activity by increasing cytoplasmic retention or sequestration of YAP. In some embodiments, the IgCAM-binding agent is an antagonist of YAP activity by decreasing nuclear translocation of YAP. In some embodiments, the IgCAM-binding agent is an antagonist of YAP activity by increasing degradation of YAP. In certain embodiments, the IgCAM-binding agent inhibits YAP activity by at least about 10%, at least about 20%, at least about 30%, at least about 50%, at least about 75%, at least about 90%, or about 100%.

In certain embodiments, the IgCAM-binding agent (e.g., an antibody or a soluble receptor) is an antagonist of TAZ activity. In some embodiments, the IgCAM-binding agent is an antagonist of TAZ activity by increasing phosphorylation of TAZ. In some embodiments, the IgCAM-binding agent is an antagonist of TAZ activity by increasing cytoplasmic retention or sequestration of TAZ. In some embodiments, the IgCAM-binding agent is an antagonist of TAZ activity by decreasing nuclear translocation of TAZ. In some embodiments, the IgCAM-binding agent is an antagonist of TAZ activity by increasing degradation of TAZ. In certain embodiments, the IgCAM-binding agent inhibits TAZ activity by at least about 10/, at least about 20%, at least about 30%, at least about 50%, at least about 75%, at least about 90%, or about 100%. In certain embodiments, the IgCAM-binding agent (e.g., an antibody or a soluble receptor) increases Hippo pathway signaling. In certain embodiments, Hippo pathway signaling by one or more IgCAMs selected from the group consisting of: AMICA, CAR, CLMP, ESAM, GPA33, VSIG1, VSIG2, VSIG3, VSIG4, VSIG5, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25 is increased. In certain embodiments, the increase of Hippo pathway signaling by an IgCAM-binding agent results in an increase in the level of Hippo pathway signaling of at least about 10%, at least about 25%, at least about 50%, at least about 75%, at least about 90%, or at least about 95%. In certain embodiments, the increase of Hippo pathway signaling by an IgCAM-binding agent results in an increase in the amount of phosphorylated YAP of at least about 10%, at least about 25%, at least about 50%, at least about 75%, at least about 90%, or at least about 95%. In certain embodiments, the increase of Hippo pathway signaling by an IgCAM-binding agent results in a decrease in the amount of non-phosphorylated YAP of at least about 10%, at least about 25%, at least about 50%, at least about 75%, at least about 90°, or at least about 95%.

In certain embodiments, the increase of Hippo pathway signaling by an IgCAM-binding agent results in an increase in the amount of phosphorylated TAZ of at least about 10%, at least about 25%, at least about 50%, at least about 75%, at least about 90′/, or at least about 95%. In certain embodiments, the increase of Hippo pathway signaling by an IgCAM-binding agent results in a decrease in the amount of non-phosphorylated TAZ of at least about 10%, at least about 25%, at least about 50%, at least about 75%, at least about 90%, or at least about 95%.

In certain embodiments, the IgCAM-binding agent (e.g., an antibody or soluble receptor) inhibits activation of YAP and/or TAZ. It is understood that an IgCAM-binding agent that inhibits activation of YAP and/or TAZ may, in certain embodiments, inhibit activation of YAP and/or TAZ by one or more IgCAMs, but not necessarily inhibit activation of YAP and/or TAZ by all IgCAMs. In certain embodiments, activation of YAP and/or TAZ is inhibited by one or more IgCAMs selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25. In certain embodiments, the inhibition of activation of YAP by an IgCAM-binding agent results in a reduction in the level of activation of YAP and/or TAZ of at least about 10%, at least about 25%, at least about 50%, at least about 75%, at least about 90%, or at least about 95%. In certain embodiments, the inhibition of activation of YAP and/or TAZ by an IgCAM-binding agent results in an increase in the amount of phosphorylated YAP and/or TAZ of at least about 10%, at least about 25%, at least about 50%, at least about 75%, at least about 90%, or at least about 95%. In certain embodiments, the inhibition of activation of YAP and/or TAZ by an IgCAM-binding agent results in a decrease in the amount of non-phosphorylated YAP of at least about 10%, at least about 25%, at least about 50%, at least about 75%, at least about 90%, or at least about 95%.

In certain embodiments, the IgCAM-binding agent (e.g., an antibody or a soluble receptor) reduces expression of at least one YAP-dependent gene. In certain embodiments, the IgCAM-binding agent reduces expression of at least one protein encoded by a YAP-dependent gene. In certain embodiments, the IgCAM-binding agent (e.g., an antibody or a soluble receptor) reduces expression of at least one TAZ-dependent gene. In certain embodiments, the IgCAM-binding agent reduces expression of at least one protein encoded by a TAAZ-dependent gene. In certain embodiments, one or more IgCAMs selected from the group consisting of: AMICA, CAR, CLMP, ESAM, GPA33, VSIG1, VSIG2, VSIG3, VSIG4, VSIG5, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25 reduces expression of at least one YAP-dependent gene or reduces expression of at least one TAZ-dependent gene. In some embodiments, the IgCAM-binding agent reduces expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, and AREG, FN1, ADAMTS1, AXL, ADAMTS5, MET, CYR61, IL8, ZEB1, FOXC2, N-cadherin, and SNAIL. In certain embodiments, an IgCAM-binding agent results in a reduction in the level of expression of at least one YAP-dependent gene of at least about 10%, at least about 25%, at least about 50%, at least about 75%, at least about 90%, or at least about 95%. In certain embodiments, an IgCAM-binding agent results in a reduction in the level of expression of at least one TAZ-dependent gene of at least about 10%, at least about 25%, at least about 50%, at least about 75%, at least about 90%, or at least about 95%.

In vivo and in vitro assays for determining whether an IgCAM-binding agent (or candidate IgCAM-binding agent) modulates Hippo pathway signaling are known in the art or are being developed. In some embodiments, the effect of an agent on Hippo pathway signaling is evaluated by YAP and/or TAZ activity. If the Hippo pathway is inhibited, activated, non-phosphorylated YAP and/or TAZ translocates to the nucleus of the cell where it binds with a DNA-binding transcription factor, such as TEAD leading to transcription of a variety of target genes. Therefore, for example, a cell-based, luciferase reporter assay utilizing a reporter vector containing multiple copies of the GT-IIC motif from the SV40 enhancer, which serves as a TEAD binding site upstream of a firefly luciferase reporter gene (TBS-Luc reporter) may be used to measure YAP and/or TAZ activity in vitro. If an IgCAM-binding agent activates the Hippo pathway, thereby inhibiting YAP and/or TAZ activity, luciferase activity will be inhibited. In addition to the TBS-Luc reporter assay, the effect of an IgCAM-binding agent (or candidate agent) on Hippo pathway signaling may be measured in vitro or in vivo by measuring the effect of the agent on the level of expression of YAP-dependent genes and/or TAZ-dependent genes, such as CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5, FGF1, IL33, GRB2, IGFBP3, and AREG. In certain embodiments, the effect of an IgCAM-binding agent on Hippo pathway signaling may also be assessed by measuring the effect of the agent on the phosphorylation state of Mst1, Mst2, Lats1, Lats2, YAP, and/or TAZ.

In some embodiments, modulation of the Hippo pathway signaling is evaluated by translocation of YAP. As described above, when the Hippo pathway is inhibited, activated non-phosphorylated YAP translocates to the nucleus. By tagging the YAP protein, for example with GFP, the location of YAP can be determined. Therefore, for example, a cell-based assay using a YAP-GFP fusion protein may be used to determine YAP activation by observing the location of YAP after treatment with an agent. Another assay to determine YAP activation comprises using a YAP-Cre fusion protein with a Cre-dependent fluorescent protein. For example, a monomeric form of fluorescent protein DsRed is flanked by loxP sites 5′ to a green fluorescent protein and serves as a reporter protein. YAP is fused to the coding region of Cre. When YAP is inactive and cytoplasmic, the fluorescent protein DsRed is expressed in the nucleus; however, when YAP is activated, the YAP-Cre protein translocates to the nucleus of the cell, the DsRed is removed by Cre/loxP recombination and the green fluorescent protein is expressed in the nucleus.

In some embodiments, modulation of the Hippo pathway signaling is evaluated by translocation of TAZ. As with YAP, when the Hippo pathway is inhibited, activated non-phosphorylated TAZ translocates to the nucleus. By tagging the TAZ protein, for example with GFP, the location of TAZ can be determined. Therefore, for example, a cell-based assay using a TAZ-GFP fusion protein may be used to determine TAZ activation by observing the location of TAZ after treatment with an agent. Another assay to determine TAZ activation comprises using a TAZ-Cre fusion protein with a Cre-dependent fluorescent protein. For example, a monomeric form of fluorescent protein DsRed is flanked by loxP sites 5′ to a green fluorescent protein. TAZ is fused to the coding region of Cre. When TAZ is inactive and cytoplasmic, the fluorescent protein DsRed is expressed in the nucleus; however, when TAZ is activated, the TAZ-Cre protein translocates to the nucleus of the cell, the DsRed is removed by Cre/loxP recombination and the green fluorescent protein is expressed in the nucleus.

In certain embodiments, the IgCAM-binding agents have one or more of the following effects: inhibit proliferation of tumor cells, inhibit tumor growth, reduce the tumorigenicity of a tumor, reduce the tumorigenicity of a tumor by reducing the frequency of cancer stem cells in the tumor, trigger cell death of tumor cells, increase cell contact-dependent growth inhibition, increase tumor cell apoptosis, reduce epithelial mesenchymal transition (EMT), or decrease survival of tumor cells.

In certain embodiments, the IgCAM-binding agents are capable of inhibiting tumor growth. In certain embodiments, the IgCAM-binding agents are capable of inhibiting tumor growth in vivo (e.g., in a xenograft mouse model, and/or in a human having cancer).

In certain embodiments, the IgCAM-binding agents are capable of reducing the tumorigenicity of a tumor. In certain embodiments, the IgCAM-binding agent is capable of reducing the tumorigenicity of a tumor in an animal model, such as a mouse xenograft model. In certain embodiments, the IgCAM-binding agent is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236; U.S. Patent Publication No. 2008/0064049; and U.S. Patent Publication No. 2008/0178305.

In certain embodiments, the IgCAM-binding agents increase proliferation of cells, increase wound healing, and/or increase tissue regeneration.

In certain embodiments, the IgCAM-binding agents described herein have a circulating half-life in mice, cynomolgus monkeys, or humans of at least about 5 hours, at least about 10 hours, at least about 24 hours, at least about 3 days, at least about 1 week, or at least about 2 weeks. In certain embodiments, the IgCAM-binding agent is an IgG (e.g., IgG1 or IgG2) antibody that has a circulating half-life in mice, cynomolgus monkeys, or humans of at least about 5 hours, at least about 10 hours, at least about 24 hours, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0 (see, e.g., U.S. Patent Publication Nos. 2005/0276799, 2007/0148164, and 2007/0122403). Known methods of increasing the circulating half-life of antibody fragments lacking the Fc region include such techniques as PEGylation.

In some embodiments of the present invention, the IgCAM-binding agents are polypeptides. In some embodiments of the present invention, the IgCAM-binding agents are small peptides. The polypeptides can be recombinant polypeptides, natural polypeptides, or synthetic polypeptides that bind at least one human IgCAM. It will be recognized in the art that some amino acid sequences of the invention can be varied without significant effect of the structure or function of the protein. Thus, the invention further includes variations of the polypeptides which show substantial activity against a human IgCAM. In some embodiments, amino acid sequence variations of IgCAM-binding polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 21^{st} Edition, 2005, University of the Sciences, Philadelphia, Pa.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., 1984, PNAS, 81:5662-5066 and U.S. Pat. No. 4,588,585.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding binding agents (e.g., antibodies or soluble receptors) against at least one human IgCAM. For example, recombinant expression vectors can be replicable DNA constructs which have synthetic or cDNA-derived DNA fragments encoding a polypeptide chain of an IgCAM-binding agent, an anti-IgCAM antibody, or fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of an IgCAM-binding polypeptide or IgCAM-binding antibody (or an IgCAM protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are described by Pouwels et al. (1985, Cloning Vectors: A Laboratory Manual, Elsevier, New York, N.Y.). Additional information regarding methods of protein production, including antibody production, can be found, e.g., in U.S. Patent Publication No. 2008/0187954; U.S. Pat. Nos. 6,413,746 and 6,660,501; and International Patent Publication No. WO 2004/009823.

Various mammalian or insect cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), and HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are well-known to those of skill in the art (see, e.g., Luckow and Summers, 1988, Bio/Technology, 6:47).

Thus, the present invention provides cells comprising the IgCAM-binding agents described herein. In some embodiments, the cells produce the IgCAM-binding agents described herein. In certain embodiments, the cells produce an antibody. In certain embodiments, the cells produce a fusion protein. In some embodiments, the cells produce a soluble receptor.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify an IgCAM-binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005.

In certain embodiments, the IgCAM-binding agent is a polypeptide that is not an antibody or does not comprise an antibody Fc region. In certain embodiments, the polypeptide comprises a protein scaffold of a type selected from the group consisting of protein A, protein G, a lipocalin, a fibronectin domain, an ankyrin consensus repeat domain, and thioredoxin. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify an IgCAM-binding polypeptide. In certain embodiments, mammalian cell display technology may be used to produce and/or identify an IgCAM-binding polypeptide.

In certain embodiments, the IgCAM-binding agents can be used in any one of a number of conjugated (i.e. an immunoconjugate or radioconjugate) or non-conjugated forms. In certain embodiments, the binding agents can be used in a non-conjugated form to harness the subject's natural defense mechanisms including CDC and ADCC to eliminate malignant or cancer cells.

In some embodiments, the IgCAM-binding agent (e.g., an antibody, a soluble receptor, or a polypeptide) is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated binding agent. A variety of radionuclides are available for the production of radioconjugated binding agents including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. Conjugates of a binding agent and one or more small molecule toxins, such as a calicheamicin, maytansinoids, a trichothene, and CC1065, and the derivatives of these toxins that have toxin activity, can also be used. In some embodiments, the IgCAM-binding agent (e.g., an antibody, a soluble receptor, or a polypeptide) is conjugated to a maytansinoid. Conjugates of a binding agent and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. POLYNUCLEOTIDES

In certain embodiments, the invention encompasses polynucleotides comprising polynucleotides that encode a polypeptide that specifically binds at least one human IgCAM or a fragment of such a polypeptide. The term “polynucleotides that encode a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, the polynucleotide comprises a polynucleotide encoding a polypeptide comprising an amino acid sequence selected from the group consisting of: SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, and SEQ ID NO:64.

In certain embodiments, the polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide encoding an amino acid sequence selected from the group consisting of: SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, and SEQ ID NO:64. Also provided is a polynucleotide that comprises a polynucleotide that hybridizes to a polynucleotide or to a polynucleotide complementary to a polynucleotide encoding an amino acid sequence selected from the group consisting of: SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, and SEQ ID NO:64. In certain embodiments, the hybridization is under conditions of high stringency.

In certain embodiments, the polynucleotides comprise the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments, the polynucleotides comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag, a peptide of sequence DYKDDDDK (SEQ ID NO:73) which can be used in conjunction with other affinity tags.

The present invention further relates to variants of the hereinabove described polynucleotides encoding, for example, fragments, analogs, and/or derivatives.

In certain embodiments, the present invention provides polynucleotides comprising polynucleotides having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising an IgCAM-binding agent (e.g., an antibody or a soluble receptor) described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, the polynucleotide variants contain alterations which produce silent substitutions, additions, or deletions, but do not alter the properties or activities of the encoded polypeptide. In some embodiments, nucleotide variants are produced by silent substitutions due to the degeneracy of the genetic code. Polynucleotide variants can be produced for a variety of reasons, for example, to optimize codon expression for a particular host (i.e., change codons in the human mRNA to those preferred by a bacterial host such as E. coli).

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

### IV. METHODS OF USE AND PHARMACEUTICAL COMPOSITIONS

The IgCAM-binding agents (e.g., antibodies and soluble receptors) of the invention are useful in a variety of applications including, but not limited to, therapeutic treatment methods, such as the treatment of cancer. In certain embodiments, the binding agents are useful for activating and/or enhancing Hippo pathway signaling, increasing cell contact-dependent growth inhibition, inhibiting YAP activation, inhibiting TAZ activation, inhibiting tumor growth, reducing tumor volume, increasing tumor cell apoptosis, and/or reducing the tumorigenicity of a tumor. The methods of use may be in vitro, ex vivo, or in vivo methods. In some embodiments, an IgCAM-binding agent is an agonist of the Hippo pathway. In some embodiments, an IgCAM-binding agent inhibits YAP activity. In some embodiments, an IgCAM-binding agent inhibits TAZ activity.

In some embodiments, the IgCAM-binding agents (e.g., an antibody or a soluble receptor) are used in the treatment of a disease associated with activation of YAP, activation of TAZ, decreased Hippo pathway signaling, and/or aberrant Hippo pathway signaling. In some embodiments, the IgCAM-binding agents are used in the treatment of a disease associated with aberrant expression or aberrant exposure of IgCAMs. In some embodiments, the disease is a disease dependent upon YAP activation. In some embodiments, the disease is a disease dependent upon TAZ activation. In some embodiments, the IgCAM-binding agents are used in the treatment of disorders characterized by increased levels of stem cells and/or progenitor cells. In some embodiments, the methods comprise administering a therapeutically effective amount of an IgCAM-binding agent to a subject. In some embodiments, the subject is human.

The present invention provides methods for inhibiting growth of a tumor using the IgCAM-binding agents described herein. In certain embodiments, the method of inhibiting growth of a tumor comprises contacting a cell with an IgCAM-binding agent (e.g., an antibody or a soluble receptor) in vitro. For example, an immortalized cell line or a cancer cell line is cultured in medium to which is added an IgCAM-binding agent to inhibit tumor growth. In some embodiments, tumor cells are isolated from a patient sample such as, for example, a tissue biopsy, pleural effusion, or blood sample and cultured in medium to which is added an IgCAM-binding agent to inhibit tumor growth.

In some embodiments, the method of inhibiting growth of a tumor comprises contacting the tumor or tumor cells with an IgCAM-binding agent (e.g., an antibody or a soluble receptor) in vivo. In certain embodiments, contacting a tumor or tumor cell with an IgCAM-binding agent is undertaken in an animal model. For example, an IgCAM-binding agent may be administered to immunocompromised mice (e.g. NOD/SCID mice) which have xenografts. In some embodiments, tumor cells are isolated from a patient sample such as, for example, a tissue biopsy, pleural effusion, or blood sample and injected into immunocompromised mice that are then administered an IgCAM-binding agent to inhibit tumor cell growth. In some embodiments, the IgCAM-binding agent is administered at the same time or shortly after introduction of tumorigenic cells into the animal to prevent tumor growth (“preventative model”). In some embodiments, the IgCAM-binding agent is administered as a therapeutic after tumors have grown to a specified size (“therapeutic model”). In some embodiments, the IgCAM-binding agent is an antibody. In some embodiments, the IgCAM-binding agent is a soluble receptor.

In certain embodiments, the method of inhibiting growth of a tumor comprises administering to a subject a therapeutically effective amount of an IgCAM-binding agent. In certain embodiments, the subject is a human. In certain embodiments, the subject has a tumor or has had a tumor which was removed. In some embodiments, the subject has a tumor with an elevated expression level of YAP. In some embodiments, the “elevated” expression level is in comparison to the expression level of YAP in normal tissue of the same tissue type. In some embodiments, the “elevated” expression level is in comparison to the expression level of YAP in other tumors of the same tissue type. In some embodiments, the “elevated” expression level is in comparison to the expression level of YAP in a reference sample. In some embodiments, the “elevated” expression level is in comparison to a pre-determined level of YAP. These comparisons may also be applied to the terms “increased”, “decreased”, and similar terms. In some embodiments, the subject has a tumor with an increased level of non-phosphorylated YAP. In some embodiments, the subject has a tumor with a decreased level of phosphorylated YAP. In some embodiments, the subject has a tumor with increased expression of YAP-dependent genes. In some embodiments, the subject has a tumor with an elevated expression level of TAZ. In some embodiments, the subject has a tumor with an increased level of non-phosphorylated TAZ. In some embodiments, the subject has a tumor with a decreased level of phosphorylated TAZ. In some embodiments, the subject has a tumor with increased expression of TAZ-dependent genes. In some embodiments, the IgCAM-binding agent is an antibody. In some embodiments, the IgCAM-binding agent is a soluble receptor.

In addition, the invention provides a method of inhibiting growth of a tumor in a subject, comprising administering a therapeutically effective amount of an IgCAM-binding agent to the subject. In certain embodiments, the tumor comprises cancer stem cells. In certain embodiments, the frequency of cancer stem cells in the tumor is reduced by administration of the IgCAM-binding agent. The invention also provides a method of reducing the frequency of cancer stem cells in a tumor, comprising contacting the tumor with an effective amount of an IgCAM-binding agent. In some embodiments, a method of reducing the frequency of cancer stem cells in a tumor in a subject, comprising administering to the subject a therapeutically effective amount of an IgCAM-binding agent is provided. In some embodiments, the IgCAM-binding agent is an antibody. In some embodiments, the IgCAM-binding agent is a soluble receptor.

In some embodiments, the tumor is a solid tumor. In certain embodiments, the tumor is a tumor selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic tumor. In certain embodiments, the tumor is a melanoma tumor.

In certain embodiments, the tumor is a tumor in which Hippo pathway signaling is inactive or at low levels. In some embodiments, the tumor is a tumor in which Hippo pathway signaling is aberrant. In some embodiments, the tumor or tumor cells have lost contact-dependent growth inhibition. In some embodiments, the tumor or tumor cells have enhanced anchorage-independent growth. In some embodiments, the tumor or tumor cells increased cell proliferation.

In certain embodiments, the tumor has elevated expression levels of YAP or over-expresses YAP. In general, the phrase “a tumor has elevated expression levels of” a protein (or similar phrases) refers to expression levels of a protein in a tumor as compared to expression levels of the same protein in normal tissue. In certain embodiments, the tumor has elevated levels of non-phosphorylated YAP. In some embodiments, the tumor has elevated levels of YAP in the nucleus. In general, the phrase “a tumor has elevated levels of” a protein (or similar phrases) refers to levels of a protein in a tumor as compared to levels of the same protein in normal tissue. In certain embodiments, the tumor has elevated expression levels of TAZ or over-expresses TAZ. In certain embodiments, the tumor has elevated levels of non-phosphorylated TAZ. In some embodiments, the tumor has elevated levels of TAZ in the nucleus.

The present invention further provides methods for treating cancer comprising administering a therapeutically effective amount of an IgCAM-binding agent to a subject. In certain embodiments, the cancer is characterized by cells expressing elevated levels of YAP as compared to expression levels in normal tissue. In certain embodiments, the cancer is characterized by cells expressing elevated levels of non-phosphorylated YAP as compared to expression levels in normal tissue. In certain embodiments, the cancer is characterized by cells expressing decreased levels of phosphorylated YAP as compared to expression levels in normal tissue. In some embodiments, the cancer is characterized by cells expressing increased levels of at least one YAP-dependent gene. In certain embodiments, the cancer is characterized by cells expressing elevated levels of TAZ as compared to expression levels in normal tissue. In certain embodiments, the cancer is characterized by cells expressing elevated levels of non-phosphorylated TAZ as compared to expression levels in normal tissue. In certain embodiments, the cancer is characterized by cells expressing decreased levels of phosphorylated TAZ as compared to expression levels in normal tissue. In some embodiments, the cancer is characterized by cells expressing increased levels of at least one TAZ-dependent gene. In some embodiments, the IgCAM-binding agent binds the extracellular domain of at least one human IgCAM selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25, and inhibits or reduces growth of the cancer. In some embodiments, the IgCAM-binding agent binds the extracellular domain of at least one human IgCAM selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25, inhibits YAP and/or TAZ translocation to the nucleus, and inhibits or reduces growth of the cancer. In some embodiments, the IgCAM-binding agent binds the extracellular domain of at least one human IgCAM selected from the group consisting of: AMICA, CAR, CLMP, ESAM, GPA33, VSIG1, VSIG2, VSIG3, VSIG4, VSIG5, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25, enhances phosphorylation of YAP and/or TAZ, and inhibits or reduces growth of the cancer. In some embodiments, the IgCAM-binding agent binds the extracellular domain of at least one human IgCAM selected from the group consisting of AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25, enhances YAP and/or TAZ retention in the cytoplasm, and inhibits or reduces growth of the cancer. In some embodiments, the IgCAM-binding agent binds the extracellular domain of at least one human IgCAM selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25, enhances YAP and/or TAZ degradation, and inhibits or reduces growth of the cancer. In some embodiments, the IgCAM-binding agent binds the extracellular domain of at least one human IgCAM selected from the group consisting of AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25, inhibits YAP and/or TAZ activation, and inhibits or reduces growth of the cancer. In some embodiments, the IgCAM-binding agent binds the extracellular domain of at least one human IgCAM selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25, inhibits YAP and/or TAZ activation, and reduces or inhibits the expression of at least one YAP-dependent gene or at least one TAZ-dependent gene. In some embodiments, the IgCAM-binding agent is an antibody. In some embodiments, the IgCAM-binding agent is a soluble receptor. In some embodiments, the IgCAM-binding agent does not bind CADM1.

The present invention provides for methods of treating cancer comprising administering a therapeutically effective amount of an IgCAM-binding agent to a subject (e.g., a subject in need of treatment). In certain embodiments, the subject is a human. In certain embodiments, the subject has a cancerous tumor. In certain embodiments, the subject has had a tumor removed. In some embodiments, a method of treating cancer comprises administering a therapeutically effective amount of an IgCAM-binding agent to a subject, wherein the subject has a tumor that has elevated expression of YAP and/or TAZ.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma.

In addition, the invention provides a method of reducing the tumorigenicity of a tumor in a subject, comprising administering to a subject a therapeutically effective amount of an IgCAM-binding agent. In certain embodiments, the tumor comprises cancer stem cells. In some embodiments, the tumorigenicity of a tumor is reduced by reducing the frequency of cancer stem cells in the tumor. In some embodiments, the methods comprise using the IgCAM-binding agents described herein. In certain embodiments, the frequency of cancer stem cells in the tumor is reduced by administration of an IgCAM-binding agent.

In certain embodiments, the methods further comprise a step of determining the level of YAP expression in the tumor or cancer. In some embodiments, the step of determining the level of YAP expression in the tumor or cancer comprises determining the level of expression of cytoplasmic and/or nuclear YAP. In some embodiments, the level of expression of YAP in a tumor or cancer is compared to the level of expression of YAP in a reference sample. As used herein, a “reference sample” includes but is not limited to, normal tissue, non-cancerous tissue of the same tissue type, tumor tissue of the same tissue type, and tumor tissue of a different tissue type. In some embodiments, the level of expression of YAP in a tumor or cancer is compared to a pre-determined level of YAP. In some embodiments, the level of expression of YAP in a tumor or cancer is compared to a pre-determined level of expression of YAP. In some embodiments, the level of expression of YAP in the nucleus of cells in a tumor or cancer is compared to the level of expression of YAP in the nucleus of cells in a reference sample or to a pre-determined level of YAP. In some embodiments, the level of expression of phosphorylated YAP in cells in a tumor or cancer is compared to the level of expression of phosphorylated YAP in a reference sample or to a pre-determined level of phosphorylated YAP. In some embodiments, the level of expression of non-phosphorylated YAP in cells in a tumor or cancer is compared to the level of expression of non-phosphorylated YAP in a reference sample or to a pre-determined level of non-phosphorylated YAP. In some embodiments, determining the level of YAP expression is done prior to treatment. In some embodiments, the subject is administered an IgCAM-binding agent described herein if the tumor or cancer has an elevated level of YAP expression as compared to the expression of the YAP in a reference sample or to a pre-determined level of YAP. In some embodiments, the subject is administered an IgCAM-binding agent describe herein if the tumor or cancer has an elevated level of non-phosphorylated YAP expression as compared to the expression of non-phosphorylated YAP in a reference sample or to a pre-determined level of non-phosphorylated YAP.

In certain embodiments, the methods further comprise a step of determining the level of TAZ expression in the tumor or cancer. In some embodiments, the step of determining the level of TAZ expression in the tumor or cancer comprises determining the level of expression of cytoplasmic and/or nuclear TAZ. In some embodiments, the level of expression of TAZ in a tumor or cancer is compared to the level of expression of TAZ in a reference sample. In some embodiments, the level of expression of TAZ in a tumor or cancer is compared to a pre-determined level of TAZ. In some embodiments, the level of expression of TAZ in the nucleus of cells in a tumor or cancer is compared to the level of expression of TAZ in the nucleus of cells in a reference sample or to a pre-determined level of TAZ. In some embodiments, the level of expression of phosphorylated TAZ in cells in a tumor or cancer is compared to the level of expression of phosphorylated TAZ in a reference sample or to a pre-determined level of phosphorylated TAZ. In some embodiments, the level of expression of non-phosphorylated TAZ in cells in a tumor or cancer is compared to the level of expression of non-phosphorylated TAZ in a reference sample or to a pre-determined level of non-phosphorylated TAZ. In some embodiments, determining the level of TAZ expression is done prior to treatment. In some embodiments, the subject is administered an IgCAM-binding agent described herein if the tumor or cancer has an elevated level of TAZ expression as compared to the expression of the TAZ in a reference sample or to a pre-determined level of TAZ. In some embodiments, the subject is administered an IgCAM-binding agent describe herein if the tumor or cancer has an elevated level of non-phosphorylated TAZ expression as compared to the expression of non-phosphorylated TAZ in a reference sample or to a pre-determined level of non-phosphorylated TAZ.

In certain embodiments, the methods further comprise a step of determining the level of expression of YAP-dependent genes in the tumor or cancer. In some embodiments, the level of expression of YAP-dependent genes in a tumor or cancer is compared to the level of expression of YAP-dependent genes in a reference sample or to a pre-determined level. In some embodiments, the methods further comprise a step of determining the level of expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, AREG, FN1, ADAMTS1, AXL, ADAMTS5, MET, CYR61, IL8, ZEB1, FOXC2, N-cadherin, and SNAIL.

In certain embodiments, the methods further comprise a step of determining the level of expression of TAZ-dependent genes in the tumor or cancer. In some embodiments, the level of expression of TAZ-dependent genes in a tumor or cancer is compared to the level of expression of TAZ-dependent genes in a reference sample or to a pre-determined level. In some embodiments, the methods comprise a step of determining the level of expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, AREG, FN1, ADAMTS1, AXL, ADAMTS5, MET, CYR61, IL8, ZEB1, FOXC2, N-cadherin, and SNAIL.

In addition, the present invention provides methods of identifying a human subject for treatment with an IgCAM-binding agent, comprising determining if the subject has a tumor that has an elevated level of non-phosphorylated YAP as compared to expression of non-phosphorylated YAP in a reference sample or to a pre-determined level of non-phosphorylated YAP. In some embodiments, if the tumor has an elevated level of non-phosphorylated YAP, the subject is selected for treatment with an IgCAM-binding agent that specifically binds at least one IgCAM. In some embodiments, if selected for treatment, the subject is administered an IgCAM-binding agent described herein. In certain embodiments, the subject has had a tumor removed. In some embodiments, methods of identifying a human subject for treatment with an IgCAM-binding agent, comprising determining if the subject has a tumor that has an elevated level of YAP activity as compared to expression of YAP activity in a reference sample or to a pre-determined level of YAP activity are provided. In some embodiments, methods of identifying a human subject for treatment with an IgCAM-binding agent, comprising determining if the subject has a tumor that has increased expression of YAP-dependent genes as compared to expression of YAP-dependent genes in a reference sample or to a pre-determined level are provided.

Additionally, the present invention provides methods of identifying a human subject for treatment with an IgCAM-binding agent, comprising determining if the subject has a tumor that has an elevated level of non-phosphorylated TAZ as compared to expression of non-phosphorylated TAZ in a reference sample or to a pre-determined level of non-phosphorylated TAZ. In some embodiments, if the tumor has an elevated level of non-phosphorylated TAZ, the subject is selected for treatment with an IgCAM-binding agent that specifically binds at least one IgCAM. In some embodiments, if selected for treatment, the subject is administered an IgCAM-binding agent described herein. In certain embodiments, the subject has had a tumor removed. In some embodiments, methods of identifying a human subject for treatment with an IgCAM-binding agent, comprising determining if the subject has a tumor that has an elevated level of TAZ activity as compared to expression of TAZ activity in a reference sample or to a pre-determined level of TAZ activity are provided. In some embodiments, methods of identifying a human subject for treatment with an IgCAM-binding agent, comprising determining if the subject has a tumor that has increased expression of TAZ-dependent genes as compared to expression of TAZ-dependent genes in a reference sample or to a pre-determined level are provided. The present invention also provides methods of treating cancer in a human subject, comprising: (a) selecting a subject for treatment based, at least in part, on the subject having a cancer that has an elevated level of non-phosphorylated YAP and/or has an elevated level of non-phosphorylated TAZ, and (b) administering to the subject a therapeutically effective amount of an IgCAM-binding agent described herein.

Methods for determining the level of YAP and/or TAZ expression in a cell, tumor, or cancer are known by those of skill in the art. For nucleic acid expression these methods include, but are not limited to, PCR-based assays, microarray analyses, and nucleotide sequencing (e.g., NextGen sequencing). For protein expression, these methods include, but are not limited, Western blot analysis, protein arrays, immunohistochemistry (IHC) assays, ELISAs, and FACS.

Methods for determining whether a tumor or cancer has an elevated level of YAP and/or TAZ expression can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

Methods of treating a disease or disorder in a subject, wherein the disease or disorder is associated with aberrant (e.g., decreased levels) Hippo pathway signaling are further provided. In some embodiments, the treatment methods comprise administering a therapeutically effective amount of an IgCAM-binding agent to the subject. In some embodiments, the IgCAM-binding agent is an antibody. In some embodiments, the IgCAM-binding agent is a soluble receptor.

The invention also provides a method of activating or enhancing Hippo pathway signaling in a cell comprising contacting the cell with an effective amount of an IgCAM-binding agent. In certain embodiments, the cell is a tumor cell. In certain embodiments, the method is an in vivo method wherein the step of contacting the cell with the IgCAM-binding agent comprises administering a therapeutically effective amount of the IgCAM-binding agent to the subject. In some embodiments, the method is an in vitro or ex vivo method. In certain embodiments, the IgCAM-binding agent activates, induces, enhances, and/or increases Hippo pathway signaling. In some embodiments, the IgCAM-binding agent inhibits activation of YAP. In some embodiments, the IgCAM-binding agent inhibits activation of TAZ. In some embodiments, the IgCAM-binding agent is an antibody. In some embodiments, the IgCAM-binding agent is a soluble receptor.

The invention also provides a method of inactivating or inhibiting Hippo pathway signaling in a cell comprising contacting the cell with an effective amount of an IgCAM-binding agent. In certain embodiments, the method is an in vivo method wherein the step of contacting the cell with the IgCAM-binding agent comprises administering a therapeutically effective amount of the IgCAM-binding agent to the subject. In some embodiments, the method is an in vitro or ex vivo method. In certain embodiments, the IgCAM-binding agent inhibits, suppresses, and/or decreases Hippo pathway signaling. In some embodiments, the IgCAM-binding agent increases activation of YAP. In some embodiments, the IgCAM-binding agent increases activation of TAZ. In some embodiments, the IgCAM-binding agent is an antibody. In some embodiments, the IgCAM-binding agent is a soluble receptor.

In another aspect, the invention provides methods of treating a wound, and/or promoting or enhancing wound healing in a subject. In some embodiments, the methods comprise administering to a subject a therapeutically effective amount of an IgCAM-binding agent that modulates Hippo pathway signaling. In some embodiments, the IgCAM-binding agent is an antagonist of Hippo pathway activity, suppresses Hippo pathway signaling, increases YAP activity, suppresses phosphorylation of YAP, suppresses degradation of YAP, and/or promotes activation of YAP. In some embodiments, the administered binding agent increases TAZ activity, suppresses phosphorylation of TAZ, suppresses degradation of TAZ, and/or promotes activation of TAZ. In some embodiments, the wound is an acute wound. In some embodiments, the wound is a surgical wound. In some embodiments, the wound is a chronic cutaneous wound.

In another aspect, the invention provides methods of regenerating tissue. In some embodiments, the methods comprise contacting a cell with an effective amount of an IgCAM-binding agent that modulates Hippo pathway signaling. In some embodiments, the methods comprise administering to a subject a therapeutically effective amount of an IgCAM-binding agent that modulates Hippo pathway signaling. In some embodiments, the IgCAM-binding agent is an antagonist of Hippo pathway activity, suppresses Hippo pathway signaling, increases YAP activity, suppresses phosphorylation of YAP, suppresses degradation of YAP, and/or promotes activation of YAP. In some embodiments, the IgCAM-binding agent increases TAZ activity, suppresses phosphorylation of TAZ, suppresses degradation of TAZ, and/or promotes activation of TAZ. In some embodiments, the method involves the treatment of tissue damage caused by: immune related disorders (such as autoimmune disorders); inflammation (including both acute and chronic inflammatory disorders); ischemia (such as myocardial infarction); traumatic injury (such as burns, lacerations, and abrasions); infection (such as bacterial, viral, and fungal infections); and chronic disease (such as cirrhosis of the liver).

In another aspect, the invention provides methods of targeting tumor cells with an IgCAM-binding agent, such as an agent described herein. Over-expression of IgCAMs on tumor cells or aberrant exposure of IgCAMs on tumor cells, may allow the IgCAMs to serve as targets for surveillance or immunosurveillance by the binding agents described herein. For example, within most normal cellular architecture IgCAMs would be expressed at the intercellular surfaces and would not be detected by IgCAM-binding agents. However, a tumor which has lost normal cellular architecture may comprise cells with aberrant exposure of IgCAMs, making these cells detectable by surveillance with IgCAM-binding agents. As the IgCAM-binding agents can be conjugated to cytotoxic agents, the aberrant exposure and/or expression of IgCAMs on tumor cells may also allow for targeted delivery of cytotoxins to the tumor cells. Thus, in some embodiments, the methods comprise administering to a subject an effective amount of an IgCAM-binding agent. In some embodiments, the binding agent is an antibody. In some embodiments, the IgCAM-binding agent is a soluble receptor. In some embodiments, the IgCAM-binding agent is conjugated to or complexed with a cytotoxic agent.

The present invention further provides pharmaceutical compositions comprising the IgCAM-binding agents described herein. In certain embodiments, the pharmaceutical compositions further comprise a pharmaceutically acceptable vehicle. These pharmaceutical compositions find use in inhibiting tumor growth and treating cancer in a subject (e.g., a human patient).

In certain embodiments, formulations are prepared for storage and use by combining a purified binding agent of the present invention with a pharmaceutically acceptable vehicle (e.g., a carrier or excipient). Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 21st Edition, 2005, University of the Sciences in Philadelphia, Pa.).

The pharmaceutical compositions of the present invention can be administered in any number of ways for either local or systemic treatment Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The IgCAM-binding agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 21st Edition, 2005, University of the Sciences in Philadelphia, Pa.

In certain embodiments, pharmaceutical formulations include an IgCAM-binding agent of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations can be produced. Suitable examples of sustained-release preparations include semi-permeable matrices of solid hydrophobic polymers containing an IgCAM-binding agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In certain embodiments, in addition to administering an IgCAM-binding agent, the method or treatment further comprises administering at least one additional therapeutic agent. An additional therapeutic agent can be administered prior to, concurrently with, and/or subsequently to, administration of the IgCAM-binding agent. Pharmaceutical compositions comprising an IgCAM-binding agent and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects (e.g., inhibits or kills) non-tumorigenic cells and a therapeutic agent that affects (e.g., inhibits or kills) tumorigenic CSCs.

In some embodiments, the combination of an IgCAM-binding agent and at least one additional therapeutic agent results in additive or synergistic results. In some embodiments, the combination therapy results in an increase in the therapeutic index of the IgCAM-binding agent. In some embodiments, the combination therapy results in an increase in the therapeutic index of the additional agent(s). In some embodiments, the combination therapy results in a decrease in the toxicity and/or side effects of the IgCAM-binding agent. In some embodiments, the combination therapy results in a decrease in the toxicity and/or side effects of the additional agent(s).

Useful classes of therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor. In some embodiments, the second therapeutic agent is a platinum complex such as carboplatin or cisplatin. In some embodiments, the additional therapeutic agents are a platinum complex and a taxane.

Therapeutic agents that may be administered in combination with the IgCAM-binding agents include chemotherapeutic agents. Thus, in some embodiments, the method or treatment involves the administration of an IgCAM-binding agent of the present invention in combination with a chemotherapeutic agent or in combination with a cocktail of chemotherapeutic agents. Treatment with an IgCAM-binding agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4^{th} Edition, 2008, M. C. Perry, Editor, Lippincott, Williams & Wilkins, Philadelphia, Pa.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel.

In some embodiments, an additional therapeutic agent comprises an agent such as a small molecule. For example, treatment can involve the combined administration of an IgCAM-binding agent of the present invention with a small molecule that acts as an inhibitor against tumor-associated antigens including, but not limited to, EGFR, HER2 (ErbB2), and/or VEGF. In some embodiments, an IgCAM-binding agent of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the mTOR/AKR pathway.

In some embodiments, an additional therapeutic agent comprises a biological molecule, such as an antibody. For example, treatment can involve the combined administration of an IgCAM-binding agent of the present invention with other antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits β-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

Furthermore, treatment with an IgCAM-binding agent described herein can include combination treatment with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician.

In some embodiments, for example, for tissue regeneration, the IgCAM-binding agent can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β, TNF-α, VEGF, PlGF, IL-1, IL-2, IL-3, IL-4, IL-5, IL-6 and IL-7.

In certain embodiments, the treatment involves the administration of an IgCAM-binding agent of the present invention in combination with radiation therapy. Treatment with an IgCAM-binding agent can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously.

It will be appreciated that the combination of an IgCAM-binding agent and at least one additional therapeutic agent may be administered in any order or concurrently. In some embodiments, the IgCAM-binding agent will be administered to patients that have previously undergone treatment with a second therapeutic agent. In certain other embodiments, the IgCAM-binding agent and a second therapeutic agent will be administered substantially simultaneously or concurrently. For example, a subject may be given an IgCAM-binding agent (e.g., an antibody) while undergoing a course of treatment with a second therapeutic agent (e.g., chemotherapy). In certain embodiments, an IgCAM-binding agent will be administered within 1 year of the treatment with a second therapeutic agent. In certain alternative embodiments, an IgCAM-binding agent will be administered within 10, 8, 6, 4, or 2 months of any treatment with a second therapeutic agent. In certain other embodiments, an IgCAM-binding agent will be administered within 4, 3, 2, or 1 weeks of any treatment with a second therapeutic agent. In some embodiments, an IgCAM-binding agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of an IgCAM-binding agent of the present invention depends on the type of disease to be treated, the severity and course of the disease, the responsiveness of the disease, whether the IgCAM-binding agent is administered for therapeutic or preventative purposes, previous therapy, the patient's clinical history, and so on, all at the discretion of the treating physician. The IgCAM-binding agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can easily determine optimum dosages, dosing methodologies, and repetition rates. In certain embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the IgCAM-binding agent is from about 0.1 mg to about 20 mg/kg of body weight. In certain embodiments, dosage can be given once or more daily, weekly, monthly, or yearly. In certain embodiments, the IgCAM-binding agent is given once every week, once every two weeks or once every three weeks.

In some embodiments, an IgCAM-binding agent may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

Thus, the present invention provides methods of treating cancer in a subject comprising using an intermittent dosing strategy for administering one or more agents, which may reduce side effects and/or toxicities associated with administration of an IgCAM-binding agent, chemotherapeutic agent, etc. In some embodiments, a method for treating cancer in a human subject comprises administering to the subject a therapeutically effective dose of an IgCAM-binding agent in combination with a therapeutically effective dose of a chemotherapeutic agent, wherein one or both of the agents are administered according to an intermittent dosing strategy. In some embodiments, the intermittent dosing strategy comprises administering an initial dose of an IgCAM-binding agent to the subject, and administering subsequent doses of the IgCAM-binding agent about once every 2 weeks. In some embodiments, the intermittent dosing strategy comprises administering an initial dose of an IgCAM-binding agent to the subject, and administering subsequent doses of the IgCAM-binding agent about once every 3 weeks. In some embodiments, the intermittent dosing strategy comprises administering an initial dose of an IgCAM-binding agent to the subject, and administering subsequent doses of the IgCAM-binding agent about once every 4 weeks. In some embodiments, the IgCAM-binding agent is administered using an intermittent dosing strategy and the chemotherapeutic agent is administered weekly.

### V. SCREENING

The present invention provides screening methods to identify agents that modulate the Hippo pathway. In some embodiments, the present invention provides methods for screening candidate agents, including but not limited to, proteins, peptides, peptidomimetics, small molecules, compounds, or other drugs, which modulate Hippo pathway activity.

In some embodiments, a method of screening for a candidate agent that modulates the Hippo pathway comprises determining if the agent has an effect on Hippo pathway components. In some embodiments, a method of screening for a candidate agent that modulates the Hippo pathway comprises determining if the agent has an effect on YAP activity. In some embodiments, a method of screening for a candidate agent that modulates the Hippo pathway comprises determining if the agent has an effect on TAZ activity. In some embodiments, a method of screening for a candidate agent that modulates the Hippo pathway comprises determining if the agent decreases YAP and/or TAZ activity. In some embodiments, a method of screening for a candidate agent that modulates the Hippo pathway comprises determining if the agent decreases expression of YAP-dependent genes or TAZ-dependent genes. In some embodiments, a method of screening for a candidate agent that modulates the Hippo pathway comprises determining if the agent decreases expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, AREG, FN1, ADAMTS1, AXL, ADAMTS5, MET, CYR61, IL8, ZEB1, FOXC2, N-cadherin, and SNAIL. In some embodiments, a method of screening for a candidate agent that modulates the Hippo pathway comprises determining if the agent has an effect on YAP and/or TAZ phosphorylation. In some embodiments, a method of screening for a candidate agent that modulates the Hippo pathway comprises determining if the agent increases YAP and/or TAZ phosphorylation. In some embodiments, a method of screening for a candidate agent that modulates the Hippo pathway comprises determining if the agent decreases non-phosphorylated YAP and/or non-phosphorylated TAZ. In some embodiments, a method of screening for a candidate agent that modulates the Hippo pathway comprises determining if the agent increases the level of YAP and/or TAZ in the cytoplasm. In some embodiments, a method of screening for a candidate agent that modulates the Hippo pathway comprises determining if the agent increases YAP and/or TAZ degradation. In some embodiments, a method of screening for a candidate agent that modulates the Hippo pathway comprises determining if the agent decreases the level of YAP and/or TAZ in the nucleus.

In some embodiments, a method of screening for a candidate agent that the modulates the Hippo pathway comprises: contacting a first set of cells, but not a second set of cells, with a candidate agent, and comparing the expression of YAP-dependent genes and/or TAZ-dependent genes in the first set of cells to expression of YAP-dependent genes and/or TAZ-dependent genes in the second set of cells. In some embodiments, a method of screening for a candidate agent that the modulates the Hippo pathway comprises: contacting a first set of cells, but not a second set of cells, with a candidate agent, and comparing the expression of at least one gene selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, GLI2, BIRC2, BIRC5 (survivin), FGF1, IL33, GRB2, IGFBP3, AREG, FN1, ADAMTS1, AXL, ADAMTS5, MET, CYR61, IL8, ZEB1, FOXC2, N-cadherin, and SNAIL in the first set of cells to expression of the same genes in the second set of cells. In some embodiments, a method of screening for a candidate agent that the modulates the Hippo pathway comprises: contacting a first set of cells, but not a second set of cells, with a candidate agent, and comparing the phosphorylation state of Mst1, Mst2, Lats1, Lats2, YAP, and/or TAZ in the first set of cells to the phosphorylation state of Mst1, Mst2, Lats1, Lats2, YAP, and/or TAZ in the second set of cells. In some embodiments, if the candidate agent stimulates or activates the Hippo pathway, the first set of cells has a higher level of phosphorylated YAP and/or TAZ than the second set of cells. In some embodiments, if the candidate agent stimulates or activates the Hippo pathway, the first set of cells has a decreased level of non-phosphorylated YAP and/or non-phosphorylated TAZ than the second set of cells. In some embodiments, a method of screening for a candidate agent that the modulates the Hippo pathway comprises: contacting a first set of cells, but not a second set of cells, with a candidate agent, and comparing the location (nuclear or cytoplasmic) of YAP and/or TAZ in the first set of cells to the second set of cells. In some embodiments, if the candidate agent stimulates or activates the Hippo pathway, the first set of cells has a higher level of YAP and/or TAZ in the cytoplasm than the second set of cells.

The invention also provides agents that modulate the Hippo pathway identified by any of the screening methods described herein.

### VI. KITS COMPRISING IgCAM-BINDING AGENTS

The present invention provides kits that comprise the IgCAM-binding agents described herein and that can be used to perform the methods described herein. In certain embodiments, a kit comprises at least one purified IgCAM-binding agent in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed IgCAM-binding agents of the present invention can be readily incorporated into one of the established kit formats which are well known in the art.

Further provided are kits comprising an IgCAM-binding agent as well as at least one additional therapeutic agent. In certain embodiments, the second (or more) therapeutic agent is a chemotherapeutic agent. In certain embodiments, the second (or more) therapeutic agent is an angiogenesis inhibitor.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Expression of YAP in OMP Tumors

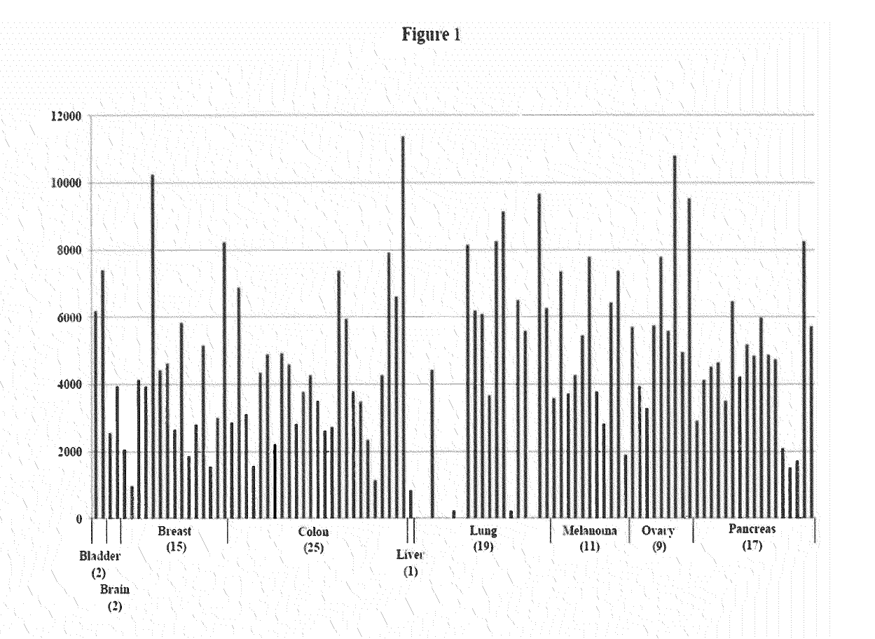

Microarray gene expression analyses of patient-derived tumors in the OncoMed tumor bank were done to determine the levels of YAP expression. 101 tumors including bladder (2), brain (2), breast (15), colon (25), liver (1), lung (19), melanoma (11), ovarian (9) and pancreas (17) were evaluated. Total RNA was isolated from these tumors that had been serially transplanted in NOD/SCID mice. RNA quality was monitored using a Bioanalyzer (Agilent, Santa Clara Calif.). The purified RNA was processed using established Affymetrix protocols. The samples were hybridized to Affymetrix HG-U133 plus 2.0 microarrays (Affymetrix, Santa Clara, Calif.) as outlined in the manufacturer's technical manuals. After hybridization, the microarrays were washed, scanned, and analyzed. Scanned array background adjustment and signal intensity normalization were performed using the GCRMA algorithm (Bioconductor, www.bioconductor.org). As shown in FIG. 1, approximate 80% of the tumors demonstrated medium to high levels of YAP expression.

### Example 2

### Inhibition of Tumor Growth by Dominant Negative YAP

A mutant YAP gene was designed and synthesized that encodes for a dominant negative human YAP (dnYAP). The dnYAP protein comprises the YAP TEAD binding domain, the two WW domains, and the PDZ domain (SEQ ID NO:68). The YAP activation domain was deleted and the serine at amino acid 127 was replaced with an alanine residue (S127A). This substitution eliminates a key phosphorylation site and allows dnYAP to translocate from the cytoplasm to the nucleus.

To express dnYAP in tumor cells both in vitro and in vivo, the dnYAP gene was cloned into a lentiviral vector containing an immediate early CMV promoter. The lentiviral vector also contains an IRES-GFP portion which allows for visualization and selection of transduced cells. The lentiviral vector is a HIV-1-derived, replication deficient, tat-independent, self-inactivating ‘3rd generation’ vector (see e.g., Durand et al., Viruses, 2011, 3:132-159). The dnYAP-expressing lentiviral vector was named pOM420. The dnYAP-expressing lentiviral vector and 3 helper plasmids were transiently transfected into HEK-293T cells to produce dnYAP lentivirus. The three helper plasmids provided the complementing functions necessary for virus production. To mediate viral entry into a wide variety of human tumor cells, the viruses were pseudotyped with the vesicular stomatitis virus envelope G protein (VSV-G).

Dissociated melanoma OMP-M6 cells (5×10⁵ cells/well) were seeded into 6-well Primaria™ plates (BD Biosciences, San Jose Calif.) and transduced with dnYAP-lentivirus (LOM420, CMV-dnYAP-IRES-GFP) or GFP-lentivirus (LOM92, CMV-IRES-GFP) as a control. Lentiviruses were added to the cells at time of plating (t=0) at a multiplicity of infection (MOI) of 2.5 infectious particles per cell. Cells were incubated overnight with the lentiviruses, washed and re-incubated in culture media. The culture medium was DMEM Low glucose, F12 medium, B27 medium, insulin-transferrin-selenium supplement, heparin, rhEGF, rhFGF-2, and antibiotics. After 72 hours, cells were collected, trypsinized, washed, and resuspended in HBSS containing 2% FBS and 4′,6-diamidino-2-phenylindole (DAPI, Molecular Probes/Invitrogen, Grand Island N.Y.). Transduced melanoma OMP-M6 cells, identified as GFP positive and DAPI negative (GFP⁺/DAPI⁻), were sorted by flow cytometry using a FACSAria flow cytometer (BD Biosciences, San Jose Calif.). Cell population purity was confirmed by reanalysis of a fraction of the sorted cells and the cells were mixed with 50% Matrigel™ (BD Biosciences, San Jose Calif.). Control lentivirus-transduced GFP⁺/DAPI⁻ cells were subcutaneously injected into the flanks of NOD/SCID mice (200 cells/mouse, n=10). dnYAP lentivirus-transduced GFP⁺/DAPI⁻ cells were subcutaneously injected into the flanks of NOD/SCID mice (200 cells/mouse, n=8). Tumor growth was monitored for 3 months.

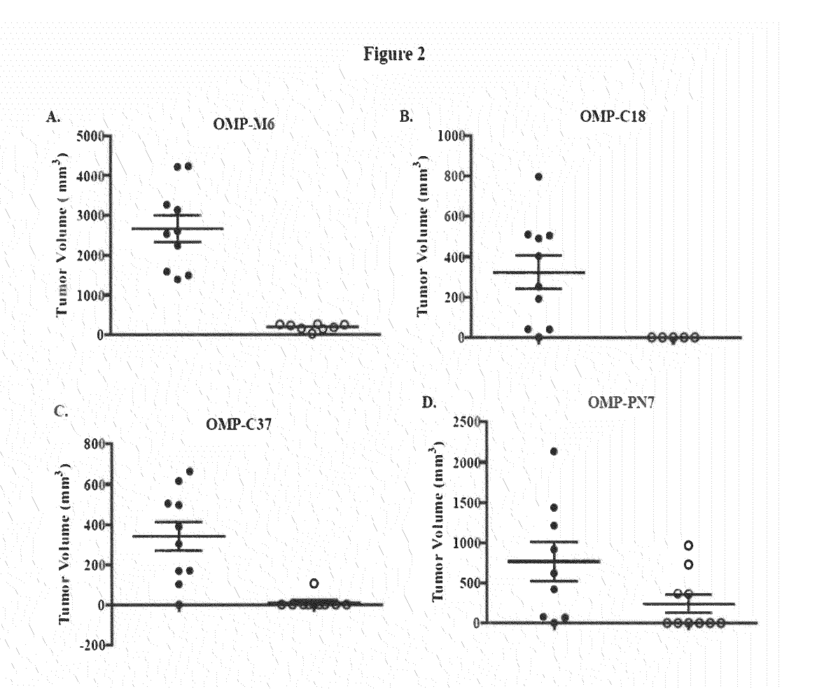

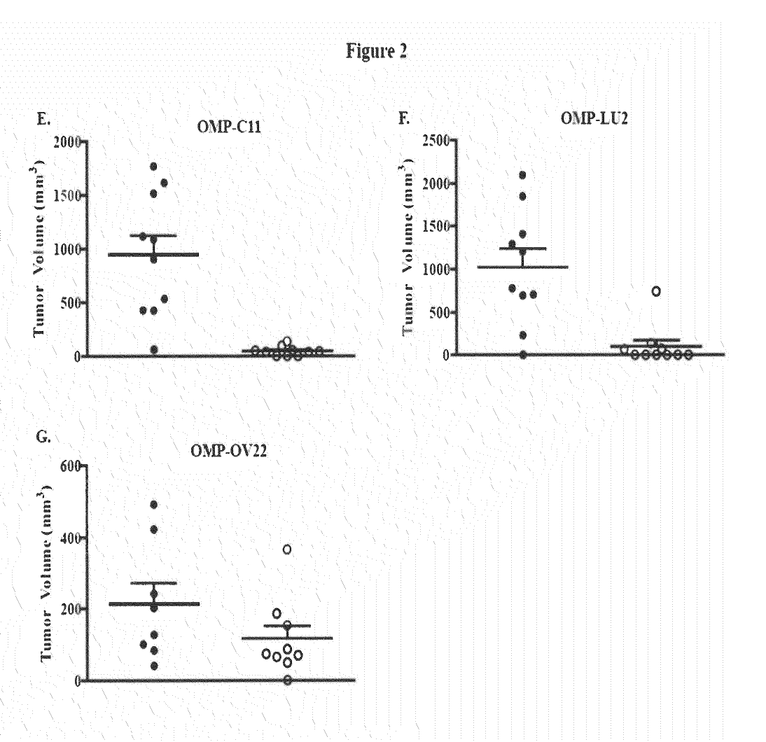

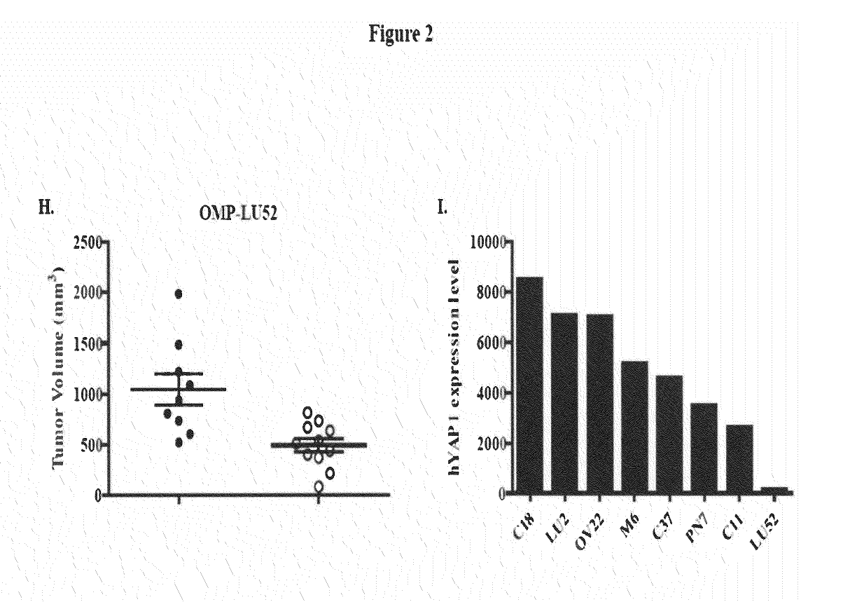

Mice injected with melanoma OMP-M6 cells expressing only GFP had palpable tumors at the site of injection approximately 40 days after injection. These tumors reached an average volume of 2000 mm³ in 2.5 months. In contrast, mice injected with melanoma OMP-M6 cells expressing dnYAP had tumors that grew extremely slowly or not at all. The growth rate of the dnYAP-expressing melanoma OMP-M6 cells was approximately 17 times slower as compared with the control group (2.8 mm³/day vs 47.5 mm³/day, respectively). This effect was noticeable and statistically significant at all measurements. As shown in FIG. 2A, on day 75 post-injection the growth of melanoma OMP-M6 tumors expressing dnYAP was significantly reduced as compared to OMP-M6 tumors expressing the GFP control (93% reduction, average tumor volume of 186 mm³ compared to an average tumor volume of 2669 mm³, respectively). These results suggest that YAP may be acting as an oncogene in melanoma, and that dnYAP was very efficient at inhibiting tumor growth.

Similar experiments were undertaken with additional tumors, including colon tumors OMP-C18, OMP-C11, OMP-C37, pancreatic tumor OMP-PN7, lung tumors OMP-LU52 and OMP-LU2, and ovarian tumor OMP-OV22. Transduced tumor cells and mouse numbers varied based on each sample's tumorigenic properties. As shown in FIG. 2B, colon tumor OMP-C18 cells transduced with dnYAP did not produce any tumors (0/5), while cells expressing the GFP control produced tumors in 9/10 mice with an average volume of 323 mm³ at day 47. As shown in FIG. 2C, colon tumor OMP-C37 cells transduced with dnYAP produced only one tumor (1/10) and this tumor was shown to be GFP negative, thus the tumor arose from cells not expressing dnYAP. In contrast, OMP-C37 tumor cells expressing the GFP control produced tumors in 9/10 mice with an average volume of 342 mm³ at day 67. As shown in FIG. 2D, pancreatic tumor OMP-PN7 cells transduced with dnYAP produced tumors in 4/10 mice. As seen with OMP-C37, the largest tumor was shown to be GFP negative, while the other 3 tumors were shown to be only 7% GFP positive, 15% GFP positive, and 41% GFP positive, indicating limited amounts of dnYAP expression. In contrast, OMP-PN7 tumor cells expressing the GFP control produced tumors in 8/9 mice with an average volume of 766 mm³ at day 80. As shown in FIG. 2E, colon tumor OMP-C11 cells transduced with dnYAP produced tumors (7/10), but all were extremely small with an average volume of 65 mm³ at day 53. In contrast, OMP-C11 tumor cells expressing the GFP control produced tumors in 10/10 mice with an average volume of 945 mm³ at day 53. As shown in FIG. 2F, lung tumor OMP-LU2 cells transduced with dnYAP produced tumors in 4/10 mice with an average tumor volume of 251 mm³ at day 87, while cells expressing the GFP control produced tumors in 9/10 mice with an average volume of 1023 mm³ at day 87. As shown in FIG. 2G, ovarian tumor OMP-OV22 cells transduced with dnYAP produced tumors in 8/9 mice, however the tumors were smaller than the control with an average volume 268 mm³. The OMP-OV22 tumor cells expressing the GFP control produced tumors in 8/8 mice with an average volume of 416 mm³ at day 108. As shown in FIG. 2H, the growth of OMP-LU52 tumors expressing dnYAP was reduced as compared to OMP-LU52 tumors expressing the GFP control at day 62 (53% reduction in tumor volume, average tumor volume of 490 mm³ compared to an average tumor volume of 1041 mm³).

These results were similar to the results observed with melanoma tumor OMP-M6, i.e., inhibition of YAP activity by dnYAP reduced or prevented tumor growth, and demonstrated that YAP may be acting as an oncogene in colon cancer, pancreatic cancer and lung cancer, as well as in melanoma. The inhibition of tumor growth by expression of dnYAP in lung tumor OMP-LU52 and ovarian tumor OMP-OV22 was less than in the other tumors studied. Interestingly, as shown by microarray analysis OMP-LU52 has a very low level of YAP expression when compared to the other tumors (FIG. 2I). This supports the idea that dnYAP is having a direct effect on YAP activity in most of the tumors studied, and that in lung tumor LU52 dnYAP had a more limited effect because there was only a small amount of YAP in the LU52 cells.

### Example 3

Microarray Analysis of Tumor Cells Expressing dnYAP

As described in Example 2, tumor cells from colon tumor OMP-C18, colon tumor OMP-C37, pancreatic tumor OMP-PN7, and lung tumor OMP-LU52 were transduced with dnYAP-lentivirus (LOM420, CMV-dnYAP-IRES-GFP) or GFP-lentivirus (LOM92, CMV-IRES-GFP) as a control. Transduced cells were cultured for 3 days and were FACS sorted for GFP expression. The gene expression profiles of these transduced tumor cells were determined by microarray analysis. RNA was isolated from the transduced cells and processed using established Affymetrix protocols. The samples were hybridized to Affymetrix HG-U133 plus 2.0 microarrays (Affymetrix, Santa Clara, Calif.) as outlined in the manufacturer's technical manuals. After hybridization, the microarrays were washed, scanned, and analyzed. Scanned array background adjustment and signal intensity normalization were performed using the GCRMA algorithm (Bioconductor, www.bioconductor.org). The gene expression profiles established for tumor cells expressing dnYAP were compared to gene expression profiles established for GFP-expressing tumor cells and analyzed using IPA software (Ingenuity Systems, Redwood City Calif.). The gene expression profiles from colon tumors OMP-C18 and OMP-C37 and pancreatic tumor OMP-PN7 expressing dnYAP showed an overlap of 208 genes that were significantly down-regulated at least 2-fold (p value≦0.05) as compared to control tumor cells. Analysis showed that the genes down-regulated by expression of dnYAP are involved in many cellular pathways and/or functions, including cell adhesion, polarity, cancer stem cells, membrane and secretory trafficking, DNA repair, RNA metabolism, mitosis, cell death, and ubiquitination. Some membrane proteins that were down-regulated in at least two of the tumors included CA12, CLDN2, PAG1, SEMA4D, GPC4, CD44, CD47, CD133, RHEB, MAGI1, ITPR3, CD168, NRP2, EPHB2 and TDGF1 and some growth factors and cytokines that were down-regulated in at least two of the tumors were AREG-B, IL33, GRB2 and IGFBP3 (Table 1). Some of the genes identified have been reported to be cancer stem cell genes and/or encode for cancer stem cell markers, for example, CD133, CD44, TDGF1, CD47, and EPHB2.

TABLE 1

| | OMP-C18 | OMP-C37 | OMP-PN7 | Lu52 |
|---|---|---|---|---|
| CD133 | −7.1 | −3.5 | −2.3 | −1.4 |
| TDGF1 | −9.2 | −3.9 | 1.0 | 1.0 |
| CD44 | −6.5 | −5.1 | −2.0 | 1.1 |
| EPHB2 | −1.9 | −1.5 | −3.8 | −1.3 |
| CD47 | −2.1 | −2.7 | −1.9 | 1.1 |
| CA12 | −3.6 | −2.2 | −4.0 | −1.2 |
| LRP4 | −4.9 | −3.7 | 1.1 | 1.1 |
| GPC4 | −2.3 | −3.3 | 1.0 | −1.0 |
| CLDN2 | −4.7 | −5.1 | −7.0 | −1.0 |
| SEMA4D | −2.2 | −2.1 | −4.6 | 1.2 |
| NRP2 | −1.5 | −2.0 | −1.8 | −1.0 |
| AREG-B | −2.3 | −2.8 | −2.6 | 1.7 |
| RHEB | −2.3 | −2.4 | 1.23 | −2.0 |
| IGFBP3 | −3.9 | −2.6 | −1.23 | 1.2 |
| CD168 | −2.0 | −2.1 | 1.77 | −1.4 |
| MAGI1 | −2.6 | −2.4 | −1.74 | 1.0 |

### Example 4

Effect of dnYAP on YAP Activity

HEK-293T cells were transiently transfected with different combinations of plasmids to evaluate the effect of dnYAP on YAP activity. Cells were transfected with a luciferase reporter comprising a promoter containing 6 copies of the GT-IIC motif of the SV40 enhancer which serves as a TEAD binding site (TBS-Luc reporter). In addition, cells were transfected with various combinations of plasmids expressing TEAD, YAP or GFP-YAP, and/or dnYAP or GFP-dnYAP. All transfections included a plasmid expressing Renilla luciferase as an internal control. 1.5×10⁵ HEK-293T cells/well were seeded into 96-well plates (BD Biosciences, San Jose Calif.) and transfected with 250 ng of an equimolar mix of the plasmids using FuGENE 6 transfection reagent (Roche Applied Science, Indianapolis Ind.) following the manufacturer's instructions. The transfected cells were incubated for 48-72 hours and luciferase activity was measured using Dual-Glo® Luciferase Assay System according to the manufacturer's instructions (Promega, Madison Wis.). Activity was expressed as a ratio of the firefly luciferase activity to the Renilla luciferase activity.

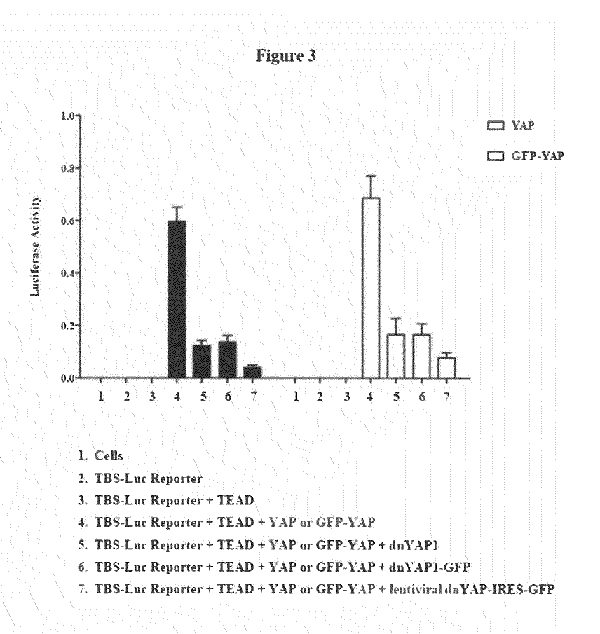

Both YAP and GFP-YAP strongly induced TEAD-mediated transcription of the TBS-Luc reporter (FIG. 3, #4). Both dnYAP and GFP-dnYAP strongly inhibited the YAP activity as demonstrated by a significant reduction in luciferase activity (FIG. 3, #5 and #6). In addition, it was demonstrated that the lentiviral vector expressing dnYAP (pOM420), which was used in in vivo tumor studies, also strongly inhibited YAP activity as demonstrated by a significant reduction in luciferase activity (FIG. 3, #7). These results demonstrated that a luciferase-based assay can be used to evaluate and/or screen molecules for their effect on YAP activity. Thus, this assay can be used to evaluate and/or screen molecules for their effect on the Hippo pathway.

To simplify the assay and to avoid transfection with multiple plasmids, HEK-293T cells were stably transfected with the TBS-Luc reporter vector and clones were selected using hygromycin B. Clones were transiently transfected with YAP and luciferase activity was measured. Two clones were identified that showed strong induction of luciferase activity after YAP expression. These stable reporter cell lines can be used to evaluate and/or screen molecules for their effect on the Hippo pathway.

### Example 5

### Effect of Cell Detachment on Hippo Pathway Components

It has been reported that cell detachment induces changes in the phosphorylation of Hippo pathway components in the MCF10A breast cell line (Zhao et al., 2012, Genes Dev., 26:54-68). A study was conducted to investigate whether cell detachment had similar effects in HEK-293T cells. HEK-293T cells were grown in culture to approximately 80% confluence, trypsinized, washed with PBS, and divided into three experimental groups. The cells in group 1 were directly lysed with tissue extraction reagent buffer (Invitrogen, Grand Island N.Y.). The cells in groups 2 and 3 were seeded into 6-well Primaria™ plates (BD Biosciences, San Jose Calif.) at a cell density of 1×10⁶ cells/well and incubated for 1 hour and 20 hours, respectively. After incubation, the attached cells were washed with PBS and directly lysed in the plate with tissue extraction reagent buffer. Proteins Lats1, Lats2, and YAP were analyzed by Western blot analysis using antibodies that recognized phosphorylated forms of the proteins and all forms of the proteins. Whole cell protein extracts were prepared with tissue extraction reagent buffer containing protease and phosphatase inhibitor cocktail (Roche Molecular, Pleasanton Calif.). Proteins were separated by SDS-polyacrylamide gel electrophoresis (PAGE) and were transferred to nitrocellulose membranes. The membranes were incubated in TBS-T (Tris-Buffered Saline plus 0.1% Tween 20) containing 5% nonfat milk to block non-specific binding. The membranes were incubated with a primary antibody overnight at 4° C. in TBS-T containing 3% BSA. Bound primary antibody was detected with an appropriate secondary HRP conjugate antibody, incubated for 1 hour at room temperature in TBS-T with 5% milk, and visualized with SuperSignal West Chemiluminescent substrate (Pierce-Thermo Fisher Scientific, Rockford Ill.). The primary antibodies used were a rabbit anti-phosphorylated YAP (pYAP) (Ser127) antibody, a rabbit anti-YAP antibody, a rabbit anti-phosphorylated Lats1 (pLats1) (Thr1079) antibody, a rabbit anti-pLats1 (Ser909) antibody, a rabbit anti-Lats1 antibody (all from Cell Signaling, Danvers Mass.), and a rabbit anti-Lats2 antibody (abcam, Cambridge Mass.). The secondary antibody used was a HRP-conjugated Affinipure donkey anti-rabbit antibody (Jackson ImmunoResearch Laboratories, West Grove Pa.). Subsequently, the membranes were stripped (Restore Plus Stripping Buffer, Pierce-Thermo Fisher Scientific, Rockford Ill.) and reprobed with a mouse anti-actin antibody (Millipore, Billerica Mass.) and a HRP-conjugated Affinipure donkey anti-mouse antibody (Jackson ImmunoResearch Laboratories, West Grove Pa.) to evaluate and confirm equivalent protein load for each sample.

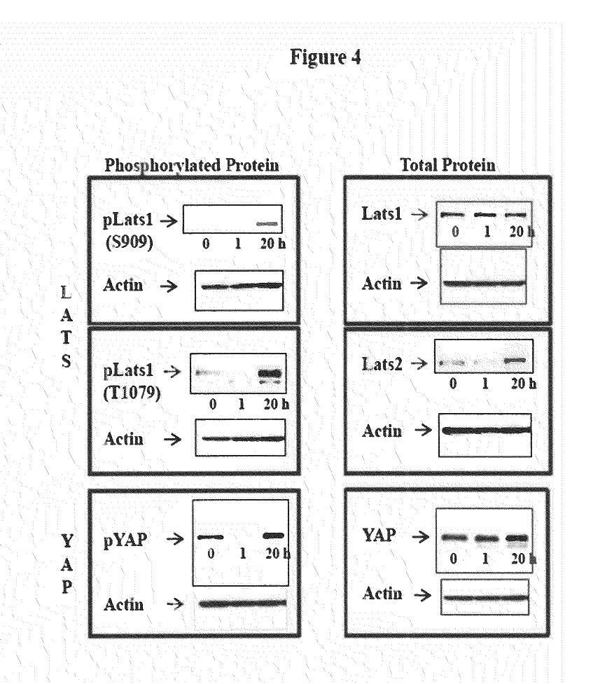

As shown in FIG. 4, the amount of pLats1 (S909 or T1079) and Lats2 proteins were significantly lower in Group 1 cells (t=0) and in Group 2 cells (t=1 hr incubation/attachment) as compared to Group 3 cells (t=20 hr incubation/attachment). No detectable reduction in total Lats1 protein was observed in any of the groups. In addition, the amount of pYAP protein was reduced in Group 1 cells (t=0) and was undetectable in Group 2 cells (t=1 hr incubation/attachment) as compared to Group 3 cells (t=20 hr incubation/attachment). Similar to results observed with Lats1, there was no reduction in total YAP in any of the groups. These results demonstrate that the Hippo pathway could be experimentally modulated in vitro in HEK-293T cells and that modification in the phosphorylation state of components of the Hippo pathway (e.g., pLats1, pYAP) can be detected.

### Example 6

IgCAM constructs

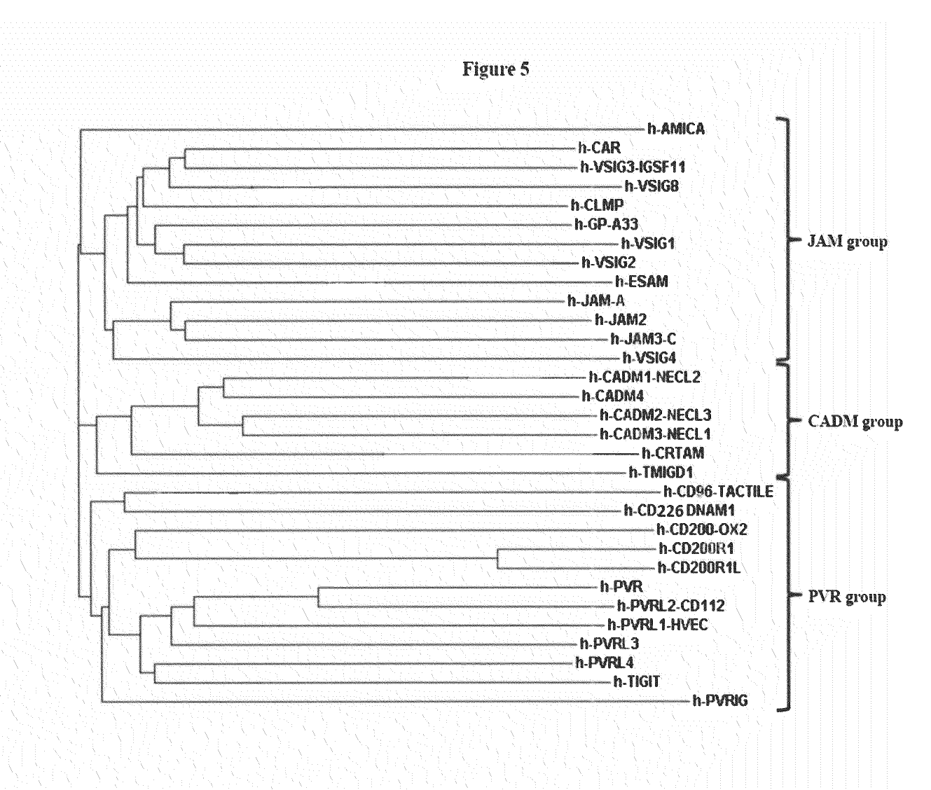

To identify potential Hippo pathway receptors a homology-based bioinformatics analysis of IgCAM proteins encoded by the human genome was undertaken. A family tree or dendrogram, of candidate Hippo pathway receptors is shown in FIG. 5. The proteins fall generally into three groups: 1) a “JAM-like” family containing JAM1, JAM2, JAM3, CAR, CLMP, AMICA, ESAM, GPA33, VSIG1, VSIG2, VSIG3, VSIG4, and VSIG8; 2) a “CADM-like” family containing CADM1, CADM2, CADM3, CADM4, CRTAM, and TMIGD1; and 3) a “PVR-like” family containing PVR, PVRL1, PVRL2, PVRL3, PVRL4, PVRIG, CD200, CD200R1, CD200R1L, CD226, CD96, and TIGIT.

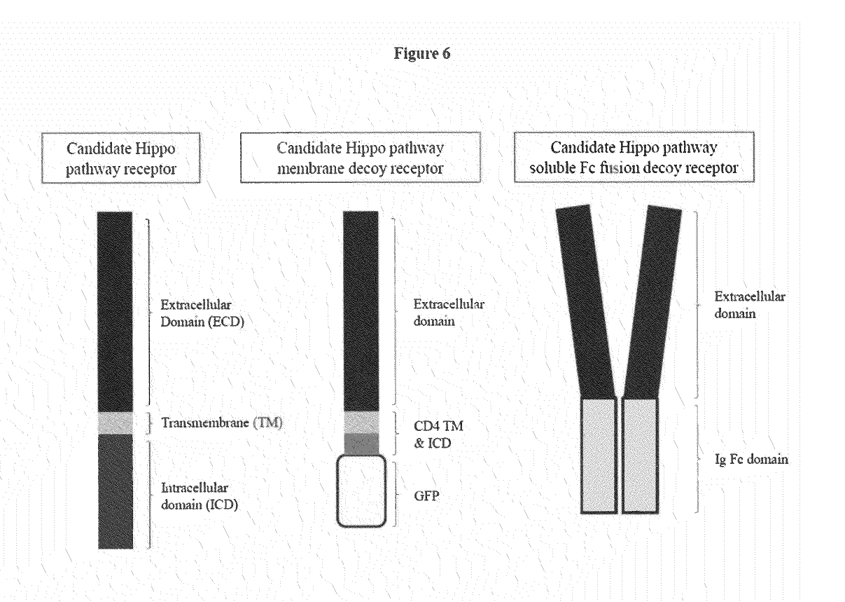

In order to investigate the possible function of these molecules in Hippo pathway signaling, IgCAM constructs were prepared including membrane-anchored proteins and soluble “decoy” receptors (FIG. 6). Each membrane-anchored decoy receptor was designed to be non-functional in regard to signaling, as the transmembrane and cytoplasmic domains were replaced with the transmembrane and intracellular domain of human CD4 protein. The membrane-anchored IgCAM protein constructs were generated by ligating the extracellular domain (ECD) region of human IgCAM proteins to the transmembrane domain and intracellular domain of CD4 and a C-terminal GFP protein tag using standard recombinant DNA techniques. These constructs are referred to as “IgCAM”-CD4TM-GFP, for example CAR-CD4TM-GFP. The soluble decoy receptors were designed to include the ECD of an IgCAM linked to an immunoglobulin Fc domain. The soluble receptor IgCAM protein constructs were generated by ligating the ECD region of human IgCAM proteins to the Fc domain of human IgG1 using standard recombinant DNA techniques. These constructs are referred to as “IgCAM”-Fc, for example CAR-Fc. As known to those of skill in the art, the ECD region of any given IgCAM protein used in the constructs may comprise the ECD or comprise a portion of the ECD. Also, what is considered to be the ECD may vary by one, two, three, or more amino acids at the amino end, the carboxyl end, or both ends of the ECD. These fusion proteins may be used to examine the role of IgCAM proteins in the Hippo pathway.

The constructs generated include ECD regions, or a portion thereof, from the IgCAM proteins in Table 2.

TABLE 2

| | | | | |
|---|---|---|---|---|
| | | | UniProtKB | |
| Name | Full name | Other names | No. | SEQ ID NO |

JAM Family

| | | | | |
|---|---|---|---|---|
| AMICA | Junctional adhesion molecule- | JAML | Q86YT9 | 1, 33 |
| | like | | | |
| CAR | Coxsackievirus and adenovirus | CXADR | P78310 | 2, 34 |
| | receptor | | | |
| CLMP | CXADR-like membrane protein | ACAM, ASAM | Q9H6B4 | 3, 35 |
| ESAM | Endothelial cell-selective | | Q96AP7 | 4, 36 |
| | adhesion molecule | | | |
| GPA33 | Cell surface A33 antigen | | Q99795 | 5, 37 |
| VSIG1 | V-set and immunoglobulin | GPA34 | Q86XK7 | 9, 41 |
| | domain-containing protein 1 | | | |
| VSIG2 | V-set and immunoglobulin | CTH, CTXL | Q96IQ7 | 10, 42 |
| | domain-containing protein 2 | | | |
| VSIG3 | Immunoglobulin superfamily | IGSF11, | Q5DX21 | 11, 43 |
| | member 11 | BTIGSF, | | |
| | | CXADRL1 | | |
| VSIG4 | V-set immunoglobulin domain- | CRIg, Z39IG | Q9Y279 | 12, 44 |
| | containing 4 | | | |
| VSIG8 | V-set and immunoglobulin | C1orf204 | Q5VU13 | 13, 45 |
| | domain-containing protein 8 | | | |
| JAM1 | Junctional adhesion molecule A | F11R, JAM-A, | Q9Y624 | 6, 38 |
| | | JCAM | | |
| JAM2 | Junctional adhesion molecule B | JAM-B, | P57087 | 7, 39 |
| | | C21orf43, | | |
| | | VEJAM | | |
| JAW3 | Junctional adhesion molecule C | JAM-C | Q9BX67 | 8, 40 |

CADM Family

| | | | | |
|---|---|---|---|---|
| CADM1 | Cell adhesion molecule 1 | IGSF4, | Q9BY67 | 14, 46 |
| | | IGSF4A, | | |
| | | NECL2, | | |
| | | SYNCAM, | | |
| | | TSLC1 | | |
| CADM2 | Cell adhesion molecule 2 | IGSF4D, | Q8N3J6 | 15, 47 |
| | | NECL3 | | |
| CADM3 | Cell adhesion molecule 3 | IGSF4B, | Q8N126 | 16, 48 |
| | | NECL1, | | |
| | | SYNCAM3, | | |
| | | TSLL1 | | |
| CADM4 | Cell adhesion molecule 4 | IGSF4C, | Q8NFZ8 | 17, 49 |
| | | NECL4, TSLL2 | | |
| CRTAM | Cytotoxic and regulatory T-cell | CD355 | Q95727 | 18, 50 |
| | molecule | | | |
| TMIGD1 | Transmembrane and | TMIGD | Q6UXZ0 | 19, 51 |
| | immunoglobulin domain- | | | |
| | containing protein 1 | | | |

PVR Family

| | | | | |
|---|---|---|---|---|
| PVR | Poliovirus receptor | NECL-5, | P15151 | 26, 58 |
| | | CD155, PVS | | |
| PVRL1 | Poliovirus receptor-related | HVEC, HLGR, | Q15223 | 27, 59 |
| | protein 1 | Nectin-1, | | |
| | | CD111, PRR1 | | |
| PVRL2 | Poliovirus receptor-related | HVEB, PRR2, | Q92692 | 28, 60 |
| | protein 2 | CD112, Nectin-2 | | |
| PVRL3 | Poliovirus receptor-related | Nectin-3, | Q9NQS3 | 29, 61 |
| | protein 3 | CD113 | | |
| PVRL4 | Poliovirus receptor-related | Nectin-4, | Q96NY8 | 30, 62 |
| | protein 4 | LNIR, PRR4 | | |
| PVRIG | Transmembrane protein PVRIG | C7orf15 | Q6DKI7 | 25, 57 |
| CD200 | OX-2 membrane glycoprotein | OX-2, MOX1, | P41217 | 21, 53 |
| | | MOX2, | | |
| CD200R1 | Cell surface glycoprotein CD200 | CD200R, | Q8TD46 | 22, 54 |
| | receptor 1 | CRTR2, | | |
| | | MOX2R, | | |
| | | OX2R | | |
| CD200R1L | Cell surface glycoprotein CD200 | CD200R2 | Q6Q8B3 | 23, 55 |
| | receptor 2 | | | |
| CD226 | CD226 antigen | DNAM1, PTA- | Q15762 | 24, 56 |
| | | 1, TLiSA1 | | |
| CD96 | T-cell surface protein tactile | | P40200 | 20, 52 |
| TIGIT | T-cell immunoreceptor with Ig | VSIG9, Vstm3, | Q495A1 | 31, 63 |
| | and ITIM domains | WUCAM | | |

Other

| | | | |
|---|---|---|---|
| TMEM25 | Transmembrane protein 25 | Q86YD3 | 32, 64 |

### Example 7

### Effect of IgCAM Proteins on Hippo Pathway Components

HEK-293T cells were transiently transfected with IgCAM constructs described in Example 6. 3.5×10⁵ HEK-293T cells/well were seeded into 6-well Primaria™ plates (BD Biosciences, San Jose Calif.). Approximately 18 hours later, 1 ug of an IgCAM-CD4TM-GFP construct (described above) was transfected into cells using FuGENE 6 transfection reagent (Roche Applied Science, Indianapolis Ind.) following the manufacturer's instructions. The IgCAM-CD4TM-GFP constructs used included CAR-CD4TM-GFP, CLMP-CD4TM-GFP, VSIG1-CD4TM-GFP, VSIG2-CD4TM-GFP, VSIG3-CD4TM-GFP, VSIG8-CD4TM-GFP, ESAM-CD4TM-GFP, JAM1-CD4TM-GFP, JAM2-CD4TM-GFP, JAM3-CD4TM-GFP, CADM1-CD4TM-GFP, CADM2-CD4TM-GFP, CADM3-CD4TM-GFP, CADM4-CD4TM-GFP, CD226-CD4TM-GFP, CD200-CD4TM-GFP, CD200R1-CD4TM-GFP, CD200R1L-CD4TM-GFP, PVR-CD4TM-GFP, PVRL1-CD4TM-GFP, PVRL2-CD4TM-GFP, PVRL3-CD4TM-GFP, PVRL4-CD4TM-GFP, TIGIT-CD4TM-GFP, TMIGD1-CD4TM-GFP, and TMEM25-CD4TM-GFP. As controls FLAG-mIgG1-CD4TM-GFP, LGR5-CD4TM, CD4TM, and RSPO4-CD4TM-GFP were used.

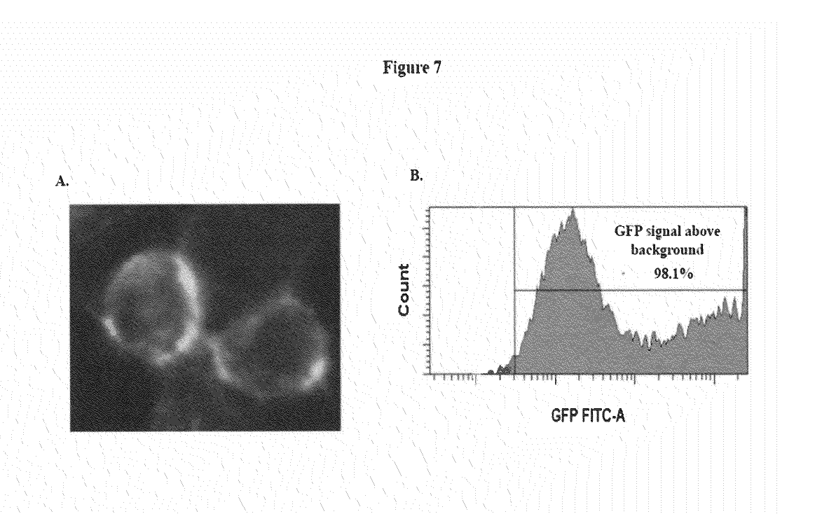

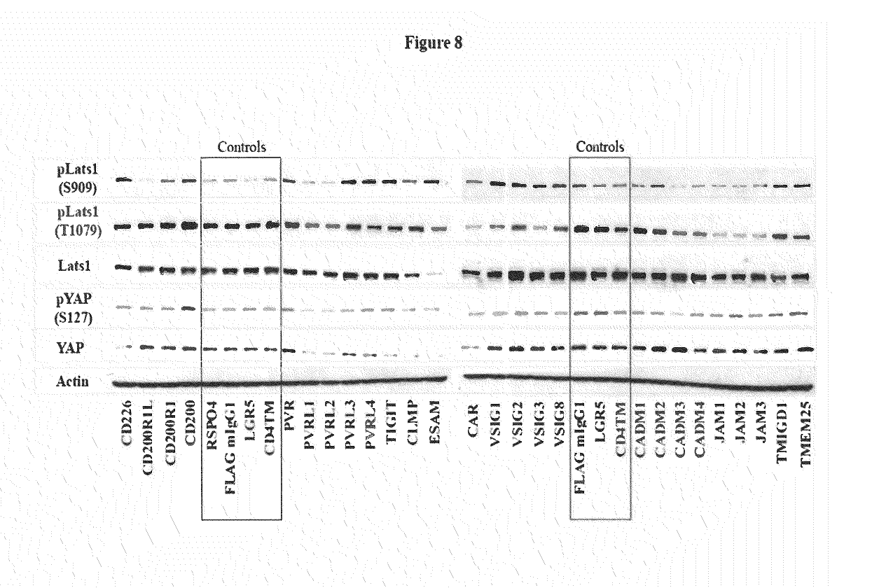

To analyze cell culture growth and GFP localization cell cultures were monitored during transient transfection using a Nikon Eclipse TS100 inverted tissue culture microscope attached to a Nikon mercury-vapor lamp and a camera. As demonstrated in FIG. 7A, which shows a representative example of transfected cells, the IgCAM-CD4TM-GFP proteins were expressed at the cell surface and were detectable by fluorescent microscopy.

After 28 hours, some transfected cells were trypsinized to obtain a single cell suspension, washed, and resuspended in HBSS containing FBS and DAPI (Molecular Probes/Invitrogen, Grand Island, N.Y.). Transfection efficiency, as determined by GFP expression levels, and viability, as determined by DAPI staining, were analyzed in a FACSCanto™ flow cytometer (BD Biosciences, San Jose Calif.). FIG. 7B shows a representative flow cytometry histogram of IgCAM-CD4TM-GFP transfected cells, demonstrating that 98% of the cells were GFP positive. For cells transfected with 26 of the IgCAM-CD4TM-GFP constructs the percentage of GFP positive cells ranged from approximately 75% to 99.5% (average 93%±6.8%). These results demonstrated that transfections using the IgCAM-CD4TM-GFP constructs were very efficient.

Other cells were directly lysed in the tissue culture plates. Whole cell protein extracts were prepared with tissue extraction reagent buffer (Invitrogen, Grand Island N.Y.) containing protease and phosphatase inhibitor cocktail (Roche Molecular, Pleasanton Calif.). Proteins were separated by SDS-PAGE and were transferred to nitrocellulose membranes. The membranes were incubated in TBS-T containing 5% nonfat milk to block non-specific binding. The membranes were incubated with primary antibodies overnight at 4° C. in TBS-T containing 3% BSA. Bound primary antibodies were detected with an appropriate secondary HRP conjugate antibody, incubated for 1 hour at room temperature in TBS-T with 5% milk, and visualized with SuperSignal West Chemiluminescent substrate (Pierce-Thermo Fisher Scientific Inc. Rockford, Ill.). The primary antibodies used were a rabbit anti-pYAP (Ser127) antibody, a rabbit anti-YAP antibody, a rabbit anti-pLats1 (Thr1079) antibody, a rabbit anti-pLats1 (Ser909) antibody, and a rabbit anti-Lats1 antibody (all from Cell Signaling, Danvers Mass.). The secondary antibody used was a HRP-conjugated Affinipure donkey anti-rabbit antibody (Jackson ImmunoResearch Laboratories, West Grove Pa.). Subsequently, the membranes were stripped using Restore Plus Stripping Buffer (Pierce-Thermo Fisher Scientific, Rockford Ill.) and reprobed with a mouse anti-actin antibody (Millipore, Billerica Mass.) and a HRP-conjugated Affinipure donkey anti-mouse antibody (Jackson ImmunoResearch Laboratories, West Grove Pa.) to evaluate and confirm equivalent protein load for each sample.

For protein expression quantification, the Western blots were scanned and the resulting images were analyzed for band intensities using densitometry software (ImageJ software, NIH). The intensity signal of each protein band was normalized to the actin signal of the same sample. Numbers in Table 3 correspond to relative protein quantity with respect to the FLAG-mIgG1 control. Positive results indicate an increased fold change with respect to control and negative results indicate a decreased fold change with respect to control.

TABLE 3

| | | | | |
|---|---|---|---|---|
| pLATS1 | pLATS1 | | | |
| S909 | T1079 | LATS1 | pYAP | YAP |

JAM Family

| | | | | | |
|---|---|---|---|---|---|
| CAR | −1.2 | −3.2 | −1.9 | −1.5 | −2.9 |
| CLMP | 2.3 | 1.3 | −2.4 | 1.3 | −10.8 |
| VSIG1 | 1.6 | −1.9 | −1.2 | 1.0 | −1.6 |
| VSIG2 | 1.3 | −1.2 | 1.3 | 1.1 | −1.1 |
| VSIG3 | −1.0 | −2.2 | −1.1 | −1.1 | −1.4 |
| VSIG8 | 1.4 | −1.2 | 1.1 | −1.3 | −1.2 |
| ESAM | 3.1 | −1.8 | −9.3 | 1.3 | −27.2 |
| JAM1 | −2.2 | −4.3 | −2.0 | −1.4 | −1.8 |
| JAM2 | −2.2 | −4.5 | −2.1 | −1.2 | −1.8 |
| JAM3 | −1.7 | −3.0 | −2.1 | −1.6 | −2.2 |

CADM Family

| | | | | | |
|---|---|---|---|---|---|
| CADM1 | −1.3 | −1.3 | −1.4 | −1.1 | −1.0 |
| CADM2 | −1.0 | −1.5 | −1.2 | −1.4 | −1.0 |
| CADM3 | −4.2 | −1.8 | −1.3 | −2.0 | −1.1 |
| CADM4 | −1.7 | −1.7 | −1.7 | −1.3 | −1.5 |
| TMIGD1 | 1.0 | −1.4 | −2.6 | −1.4 | −1.4 |

PVR Family

| | | | | | |
|---|---|---|---|---|---|
| CD226 | 3.1 | −1.2 | −2.3 | 2.1 | −3.3 |
| CD200 | 2.5 | 1.9 | −1.2 | 2.4 | 1.5 |
| CD200R1 | 1.7 | 1.3 | −1.1 | 1.6 | 1.2 |
| CD200R1L | −2.5 | −1.1 | −1.6 | 1.8 | 1.1 |
| PVR | 2.1 | 1.2 | −1.2 | 1.8 | 1.1 |
| PVRL1 | −1.2 | −1.2 | −1.3 | −1.5 | −3.6 |
| PVRL2 | −1.4 | −1.4 | −1.2 | −1.1 | −2.9 |
| PVRL3 | 3.5 | 1.4 | 1.0 | 1.4 | −1.4 |
| PVRL4 | 2.9 | 1.2 | −1.2 | 1.9 | −1.6 |
| TIGIT | 3.0 | 1.4 | −1.2 | 1.7 | −3.9 |

Other

| | | | | | |
|---|---|---|---|---|---|
| TMEM25 | 1.2 | −1.5 | −2.3 | 1.0 | −1.6 |

As shown in FIG. 8 and Table 3, expression of IgCAM-ECD constructs appeared to affect some components of the Hippo pathway. For example, pLats1 and pYAP were increased by expression of CD200-ECD as compared to controls. In contrast, pLats1 and pYAP were decreased by expression of CADM3-ECD as compared to controls. These results demonstrate that over-expression of the ECD of some IgCAM proteins, such as CD200 and CADM3, could affect components of the Hippo pathway (Lats1 and YAP) in vitro and suggest that IgCAM-binding agents could be used to modulate the Hippo pathway.

### Example 8

### Bimolecular Fluorescence Complementation Assay for Determination of YAP Nuclear Translocation

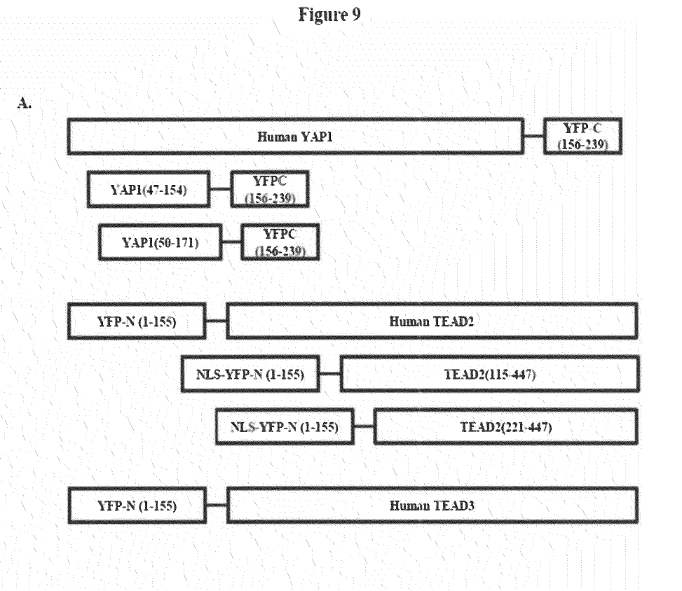

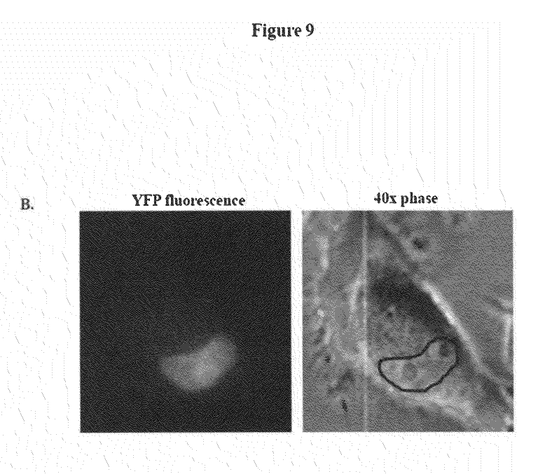

YAP localization can serve as a read-out for the activity of the Hippo pathway. Bimolecular fluorescence complementation (BiFC) constructs with human YAP1, TEAD2, and TEAD3 were generated (FIG. 9A). Specifically, the C-terminus of yellow fluorescent protein (YFP, amino acids 156-239) was fused to the C-terminus of YAP1 with a linker sequence (RPACKIPNDLKQKVMNH, SEQ ID NO:72) to generate the construct YAP1-YFPC in a pcDNA3.1(+) vector. The N-terminus of YFP (amino acids 1-155) was fused to the N-terminus of TEAD2 or TEAD3 with a linker sequence (RPACKIPNDLKQKVMNH, SEQ ID NO:72) to generate the constructs YFPN-TEAD2 and YFPN-TEAD3 in a pcDNA3.1/hygro vector. The YAP1-YFPC construct and YFPN-TEAD2 or the YAP1-YFPC construct and YFPN-TEAD3 were stably transfected into HeLa cells and double transfectants were selected using G-418 and hygromycin B. Activation of YAP should lead to predominant nuclear localization of the YAP-YFPC protein allowing for interaction with the YFPN-TEAD protein and giving rise to YFP fluorescence. Trypsinized cells from the stable clones were re-plated at low density (YAP is activated) and YFP fluorescence was evaluated by widefield epifluorescence microscopy (FIG. 9B). An additional cell line was generated, the YAP1-YFPC construct was stably transfected into HeLa cells and transfectants were selected using G-418. The HeLa YAP1-YFPC cell line can be transduced with lentiviruses expressing YFPN-TEAD2 or YFPN-TEAD3.

A variation of the BiFC assay using truncation constructs constituting the cognate binding domains of YAP1 and TEAD2 was developed based upon assays from Vassilev at al., 2001, Genes and Development, 15:1229-1241 and Li et al., 2010, Genes and Development, 24:235-240. In particular, amino acids 47-154 and 50-171 of human YAP1 were fused at their C-termini to YFPC (aa 156-239) to generate YAP1(47-154)-YFPC and YAP1(50-171)-YFPC, respectively. Furthermore, amino acids 115-447 and 221-447 of human TEAD2 were fused at their N-termini to YFPN (aa 1-115), with the YFPN domain preceded by a nuclear localization sequence, to generate NLS-YFPN-TEAD2(15-447) and NLS-YFPN-TEAD2(221-447) (FIG. 9A). As described above these constructs are stably transfected or stably transduced by lentiviral infection into HeLa cells or MCF10A cells, and the resulting cell lines are used in BiFC assays to observe YAP1 localization by widefield epifluorescence microscopy or flow cytometry. YAP1 localization is used to determine Hippo pathway signaling status.

These cell lines are used to screen and identify molecules (e.g., antibodies, proteins, or small molecules) that affect YAP activation/inactivation and potentially the Hippo pathway.

### Example 9

### Assay for Determination of YAP Nuclear Translocation

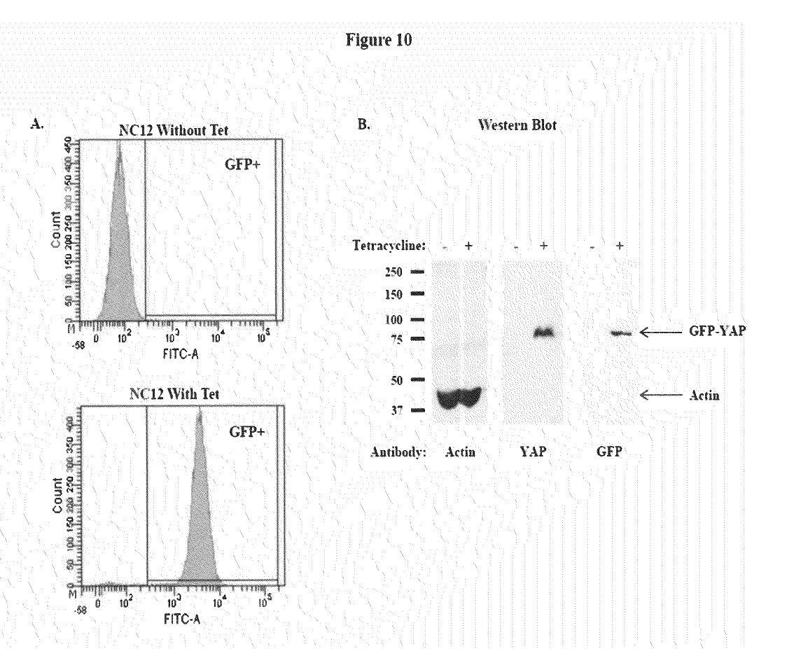

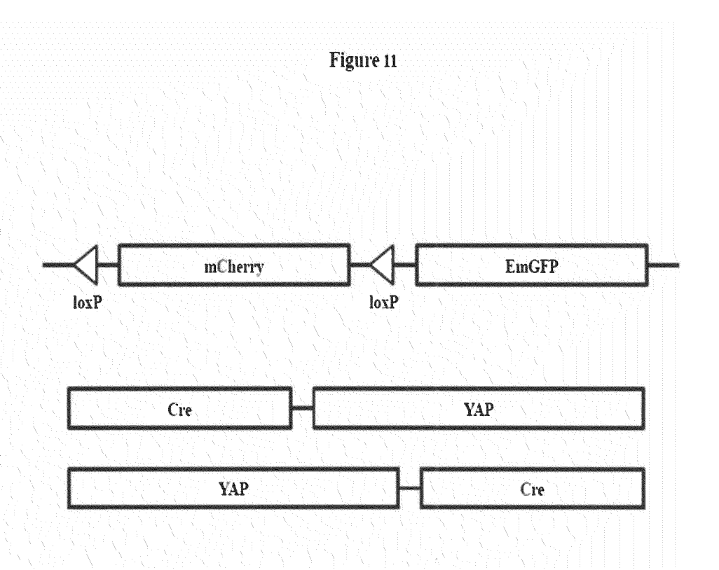

YAP localization in individual cells can also be assessed using a YAP-GFP fusion protein. The GFP allows for direct monitoring of YAP, since activation of YAP should lead to predominant nuclear localization of YAP-GFP. A YAP-GFP construct was cloned into a pLenti6.3/TO/V5-DEST vector (Invitrogen/Life Technologies, Grand Island N.Y.) generating pLenti6.3-TO-YAPGFP-DEST (pOM1017). This vector allows for tetracycline (Tet)-regulated expression of the YAP-GFP fusion protein. HeLa cells were transfected with pLenti3.3/TR repressor plasmid (Invitrogen/Life Technologies, Grand Island N.Y.) that constitutively expresses high levels of the Tet repressor under the control of a CMV promoter. Stably transfected cells were selected with 500 μg/ml of G418. Clones were screened by transiently transfecting the Tet-inducible YAP-GFP construct pOM1017 into the transfected HeLa cells and treating the cells with tetracycline. Several clones were identified that demonstrated YAP-GFP expression only in the presence of 1 μg/ml tetracycline. One of the clones, HeLaB5, was stably transfected with YAP-GFP construct pOM1017. Double transfectants were selected with 500 μg/ml G418 and 4 μg/ml of blasticidin. Clones were analyzed by FACS for the expression of GFP in the absence and presence of tetracycline. One clone, NC12, was obtained that showed tight regulation of GFP-YAP expression upon addition of 1 μg/ml tetracycline (FIG. 10A). The expression of YAP-GFP, upon Tet induction, was confirmed by Western blot analysis (FIG. 10B). The NC12 cell line can be used in assays run on a BD Pathway system which allows for real-time imaging of live cells. The assays are used to screen and identify molecules (e.g., antibodies, proteins, or small molecules) that affect YAP activation/inactivation and potentially the Hippo pathway.

### Example 10

### Assay for Determination of YAP Nuclear Translocation

YAP localization in individual cells can be assessed using a YAP-Cre fusion protein co-expressed with a Cre-dependent Emerald green fluorescent protein (EmGFP) expression construct (FIG. 1). Specifically, a monomeric form of fluorescent protein DsRed, mCherry, was flanked by loxP sites 5′ to EmGFP to generate the construct floxed mCherry/EmGFP in the vector pcDNA3.1(+) (Life Technologies, Grand Island N.Y.). YAP, under the control of the Tet operator in the vector pLenti6.3/TON/V5-DEST (Life Technologies, Grand Island N.Y.), is fused either N- or C-terminally to the coding region of Cre to generate the constructs Cre-YAP or YAP-Cre, respectively. The mCherry/EmGFP construct and Cre-YAP or mCherry/EmGFP construct and YAP-Cre were stably transfected into HeLa cells and double transfectants were selected with G-418 and blasticidin. mCherry fluorescence, arising from inactive cytoplasmic YAP-Cre or Cre-YAP, or EmGFP fluorescence, arising from active nuclear YAP-Cre or Cre-YAP, is assayed by widefield epifluorescence microscopy or flow cytometry. These cell lines are used to screen and identify molecules (e.g., antibodies, proteins, or small molecules) that affect YAP activation/inactivation and activity of the Hippo pathway.

### Example 11

Effect on Tumor Growth by Over-Expression of Membrane-Anchored IgCAM Decoy Receptors in Colon tumor OMP-C18

Dissociated colon OMP-C18 cells (1×10⁶ cells/well) were seeded into 6-well Primaria™ plates (BD Biosciences, San Jose Calif.) and transduced with RRL-based lentiviruses expressing membrane-anchored decoy receptors, CADM2-CD4TM-GFP, CADM4-CD4TM-GFP, CD226-CD4TM-GFP, PVR-CD4TM-GFP, or GFP only as a control. RRL-based lentiviruses express the GFP gene under the control of a CMV promoter. In addition, a second control was a group of cells that were not transduced. Lentiviruses were added to the cells at time of plating (t=0) at a MOI which resulted in the infection of 20% to 60% of the OMP-C18 cells. The culture medium was DMEM Low glucose, F12 medium, B27 medium, insulin-transferrin-selenium supplement, heparin, rhEGF, rhFGF-2, and antibiotics. After 72 hours, cells were collected, trypsinized, washed, and resuspended in HBSS containing 2% FBS and DAPI (Molecular Probes/Invitrogen, Grand Island N.Y.). Transduced colon OMP-C18 cells (GFP⁺/DAPI⁻) were sorted by flow cytometry using a FACSAria flow cytometer (BD Biosciences, San Jose Calif.). Cell population purity was confirmed by reanalysis of a fraction of the sorted cells. The sorted cells were mixed with 50% Matrigel™ (BD Biosciences, San Jose Calif.). Control lentivirus-transduced GFP⁺/DAPI⁻ cells were subcutaneously injected into the flanks of NOD/SCID mice (300 cells/mouse, n=10). IgCAM decoy receptor lentivirus-transduced GFP⁺/DAPI⁻ cells were subcutaneously injected into the flanks of NOD/SCID mice (300 cells/mouse, n=10). Non-transduced cells were subcutaneously injected into the flanks of NOD/SCID mice (300 cells/mouse, n=5). Tumor growth was monitored weekly, and the experiment was terminated when the fastest growing tumor reached ˜1500 mm³.

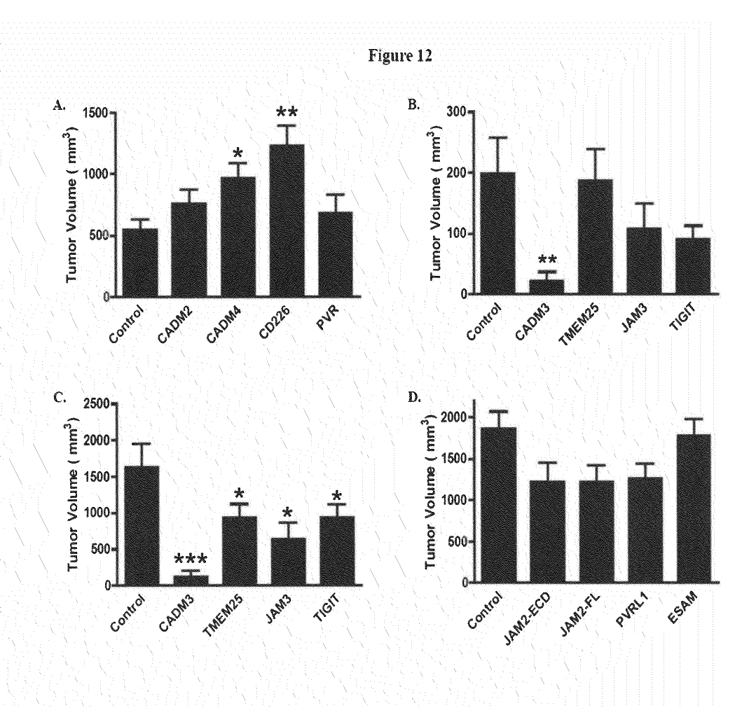

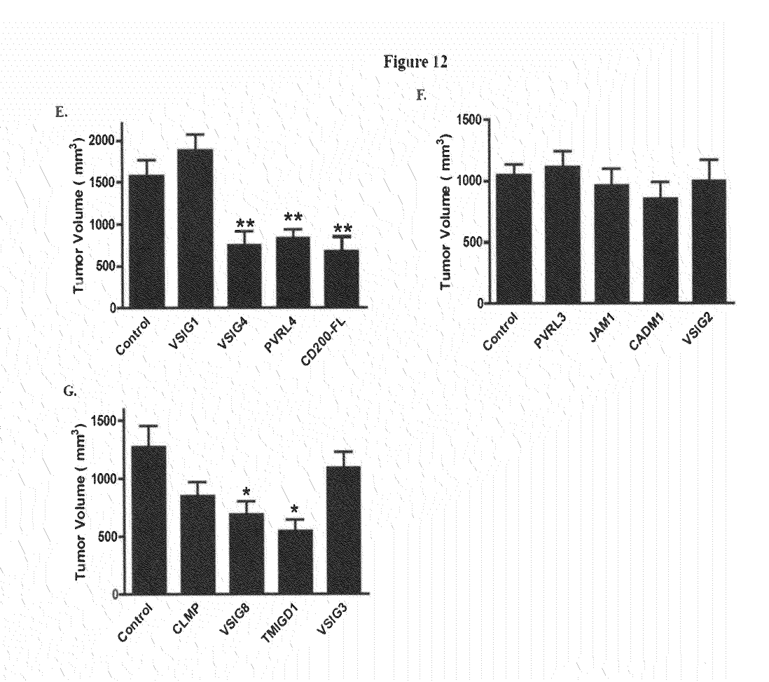

As shown in FIG. 12A, at day 45 over-expression of CADM4 and CD226 decoy receptors in OMP-C18 tumors appeared to increase tumor growth as compared to control. Over-expression of CADM2 decoy receptor also appeared to increase tumor growth as compared to control, but to a less pronounced level than CADM4 and CD226.

Similar studies were performed with OMP-C18 colon tumors and RRL-based lentiviruses expressing membrane-anchored decoy receptors, CADM3-CD4TM-GFP, TMEM25-CD4TM-GFP, JAM3-CD4TM-GFP, and TIGIT-CD4TM-GFP, (FIGS. 12B and 12C), JAM2-CD4TM-GFP, JAM2FL-GFP, PVRL1-CD4TM-GFP, and ESAM-CD4TM-GFP (FIG. 12D), VSIG1-CD4TM-GFP, VSIG4-CD4TM-GFP, PVRL4-CD4TM-GFP, and CD200-FL-GFP (FIG. 12E), PVRL3-CD4TM-GFP, JAM1-CD4TM-GFP, CADM1-CD4TM-GFP and VSIG2-CD4TM-GFP (FIG. 12F), and CLMP-CD4TM-GFP, VSIG5-CD4TM-GFP, TMIGD1-CD4TM-GFP and VSIG3-CD4TM-GFP (FIG. 12G).

As shown in FIG. 12B, at day 34 over-expression of CADM3 decoy receptor significantly inhibited growth of OMP-C18 tumor cells as compared to control. In addition, JAM3 and TIGIT appeared to inhibit tumor growth to some degree. At day 48, the inhibition of tumor growth by CADM3 as compared to control was even greater than at day 34 (FIG. 12C). Furthermore, at day 48 over-expression of TEMEM25, JAM3 and TIGIT decoy receptors significantly inhibited tumor growth as compared to control, although not to the extent observed with CADM3. At day 49, over-expression of VSIG4, PVRL4 and CD200 decoy receptors significantly inhibited growth of OMP-018 tumor cells as compared to control (FIG. 12E). At day 49, over-expression of VSIG5 and TIMIGD1 significantly inhibited growth of OMP-018 tumor cells as compared to control (FIG. 12G).

### Example 12

### Effect on Tumor Growth by Over-Expression of Membrane-Anchored IgCAM Decoy Receptors in Lung Tumors OMP-LU2 and OMP-LU40

Dissociated colon OMP-LU2 cells (1×10⁶ cells/well) were seeded into 6-well Primaria™ plates (BD Biosciences, San Jose Calif.) and transduced with RRL-based lentiviruses expressing membrane-anchored decoy receptors, CADM2-CD4TM-GFP, CADM4-CD4TM-GFP, PVRL4-CD4TM-GFP, VSIG4-CD4TM-GFP, CADM1-CD4TM-GFP, CD226-CD4TM-GFP, JAM3-CD4TM-GFP, JAM2-CD4TM-GFP, JAM2FL-GFP, CADM4-CD4TM-GFP, TIGIT-CD4TM-GFP, PVRL1-CD4TM-GFP, PVRL3-CD4TM-GFP, dnYAP as a positive control, or GFP only as a negative control. RRL-based lentiviruses express the GFP gene under the control of a CMV promoter. In addition, a second control was a group of cells that were not transduced. Lentiviruses were added to the cells at time of plating (t=0) at a MOI which resulted in the infection of 20% to 60% of the OMP-LU2 cells. The culture medium was DMEM Low glucose, F12 medium, B27 medium, insulin-transferrin-selenium supplement, heparin, rhEGF, rhFGF-2, and antibiotics. After 72 hours, cells were collected, trypsinized, washed, and resuspended in HBSS containing 2% FBS and DAPI (Molecular Probes/Invitrogen, Grand Island N.Y.). Transduced lung OMP-LU2 cells (GFP⁺/DAP⁻) were sorted by flow cytometry using a FACSAria flow cytometer (BD Biosciences, San Jose Calif.). Cell population purity was confirmed by reanalysis of a fraction of the sorted cells. The sorted cells were mixed with 50% Matrigel™ (BD Biosciences, San Jose Calif.). Control lentivirus-transduced GFP⁺/DAPI⁻ cells were subcutaneously injected into the flanks of NOD/SCID mice (300 cells/mouse, n=10). IgCAM decoy receptor lentivirus-transduced GFP⁺/DAPI⁻ cells were subcutaneously injected into the flanks of NOD/SCID mice (300 cells/mouse, n=10). Non-transduced cells were subcutaneously injected into the flanks of NOD/SCID mice (300 cells/mouse, n=5). Tumor growth was monitored weekly, and the experiment was terminated when the fastest growing tumor reached ˜1500 mm³.

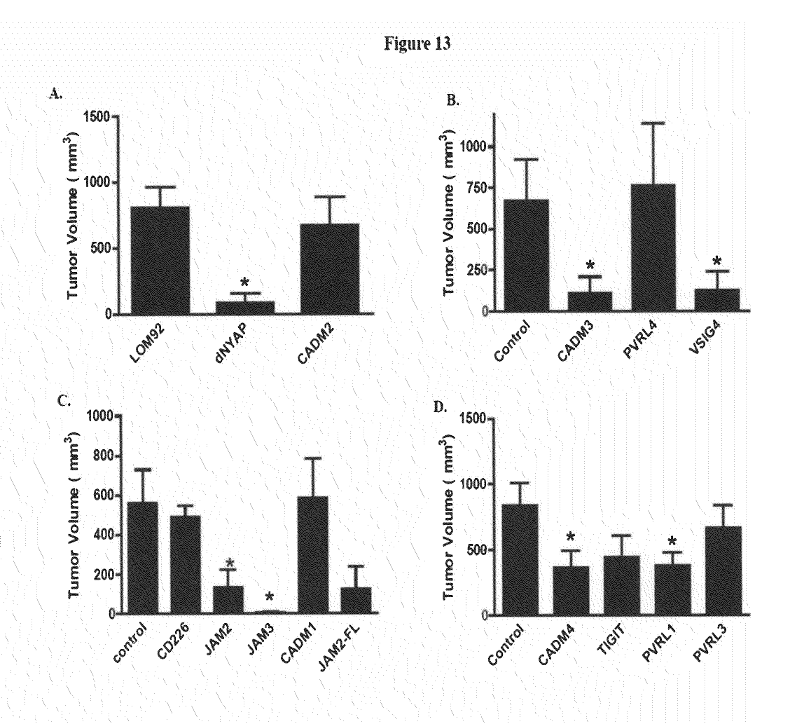

At day 108, over-expression of CADM3 and VSIG4 decoy receptors significantly inhibited growth of OMP-LU2 lung tumor cells as compared to control (FIG. 13B). At day 71, over-expression of JAM3 decoy receptor significantly inhibited growth of OMP-LU2 lung tumor cells as compared to control (FIG. 13C). In addition, at day 70 or 71, over-expression of JAM2, CADM4, and PVRL1 decoy receptors inhibited growth of OMP-LU2 tumor cells as compared to control (FIGS. 13C and 13D).

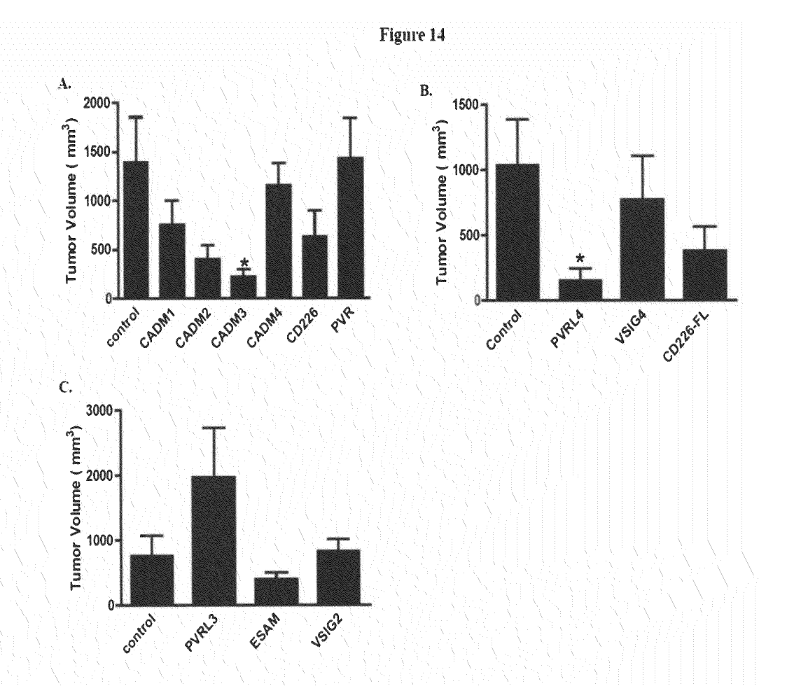

Similar studies were performed with OMP-LU40 lung tumors and RRL-based lentiviruses expressing membrane-anchored decoy receptors, CADM1-CD4TM-GFP, CADM2-CD4TM-GFP, CADM3-CD4TM-GFP, CADM4-CD4TM-GFP, CD226-CD4TM-GFP, and PVR-CD4TM-GFP (FIG. 14A), PVRL4-CD4TM-GFP, VSIG4-CD4TM-GFP, and CD226FL-GFP (FIG. 14B), PVRL3-CD4TM-GFP, ESAM-CD4TM-GFP, and VSIG2-CD4TM-GFP (FIG. 14C).

At day 118, over-expression of CADM2 and CADM3 decoy receptors significantly inhibited growth of OMP-LU40 lung tumor cells as compared to control (FIG. 13A). At day 120, over-expression of PVRL4 decoy receptor significantly inhibited growth of OMP-LU2 lung tumor cells as compared to control (FIG. 13B).

Inhibition by the decoy receptors in the studies described in Examples 11 and 12 is summarized in Table 4. The numbers represent a fold change decrease in tumor size as compared to control tumors.

TABLE 4

| | OMP-C18 | OMP-LU2 | OMP-LU40 |
|---|---|---|---|
| CLMP | 2.0 | ND | ND |
| VSIG4 | 2.4 | 3.7 | 1 |
| VSIG8 | 2.6 | ND | ND |
| JAM2 | 1.0 | 5.1 | ND |
| JAM3 | 1.7 | 56 | ND |
| CADM3 | 7.2 | 6.4 | 6.5 |
| CADM4 | Increased | 2.3 | 1 |
| PVRL1 | 1.5 | 2.2 | ND |
| PVRL4 | 1.9 | ND | 16 |
| TIGIT | 1.9 | 1.9 | ND |
| TMIGD1 | 2.3 | ND | ND |
| TMEM25 | 1.9 | ND | ND |

ND = Not determined

Fourteen IgCAMs used as membrane-anchored decoys reduced tumor growth in colon and/or lung tumors, with inhibition ranging from 1.5 to 56-fold. Among the 14 IgCAMs, CADM3, JAM3 and PVRL4 showed the greatest effect.

### Example 13

### Proteomic Studies

To investigate the signaling pathway(s) downstream of IgCAMs, a proteomic approach was taken to identify protein complexes containing a set of candidate IgCAMs implicated in either tumor growth and/or the Hippo pathway. HEK-293T cells were independently transiently transfected with FLAG-tagged full length IgCAMs, CADM1, CADM3, CD200, JAM2, PVR, PVRL1, and PVRL3. A FLAG-tagged YFP (yellow fluorescent protein) was used as a control. Four days post-transfection, lysates generated from the harvested cell pellets were clarified by centrifugation, pre-cleared with mouse IgG-agarose (Sigma-Aldrich, St. Louis, Mo.), and then incubated with anti-FLAG agarose (Sigma-Aldrich). Anti-FLAG immunoprecipitates were eluted with (2×) non-reducing Tris-Glycine SDS Sample Buffer (Life Technologies/Invitrogen, Grand Island, N.Y.). Eluates were submitted to MSBioworks (Michigan, USA, IP-Works service) for mass spectrometry to identify proteins that co-immunoprecipitate with the transfected “bait” IgCAMs.

The proteins that co-immunoprecipitated with the transfected “bait” IgCAMs were first analyzed for heterotypic interactions with endogenous IgCAMs. For example, endogenous JAM3 co-immunoprecipitates with JAM2, which corroborates results observed with FACS-binding assays. While the FACS-binding assays detected some interactions that were not found by mass spectrometry (i.e., the interaction between JAM2 and CADM3), the inverse is also true (i.e., the interaction between PVR and CD200).

Next, the list of 1039 identified proteins was annotated for function; categories of interest included 1) cytoskeleton/motors, 2) transcription, 3) kinase/phosphatase/signaling cascade, 4) miscellaneous enzymatic activity, 5) small/heterotrimeric G proteins, 6) scaffolding/protein complexes, and 7) receptor/surface protein/adhesion/IgCAM-related. The g:Profiler database (http://biit.cs.ut.ee/gprofiler) was used to analyze the protein-protein interaction networks between the selected list of 125 proteins identified in the 7 categories above and the protein components of the Hippo pathway. The 125 proteins were also analyzed by Pearson correlation to identify clusters of the “bait” IgCAMs that form complexes with the same set of proteins. Based on these two analyses, CADM1, CADM3, PVRL1, and PVRL3 were identified as IgCAMs that co-immunoprecipitated with a set of proteins localized to the membrane and/or cell-cell junctions. These proteins, some of which can interact with Hippo components such as Angiomotin or Merlin, include DCAF7, DDB1, DLG1, INADL, LIN7C, MPDZ, MPP5, MPP7, PARD3, and PRKCI.

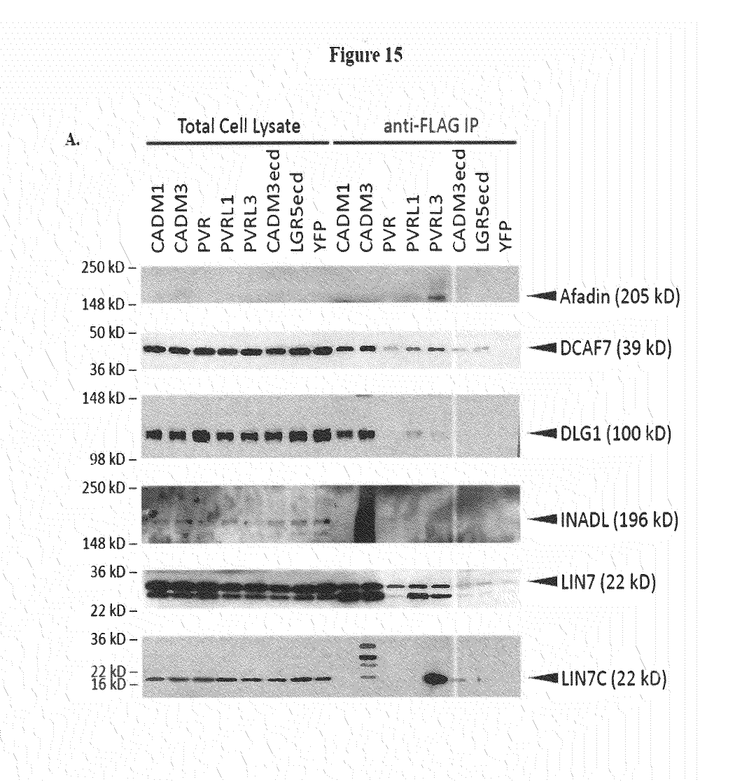

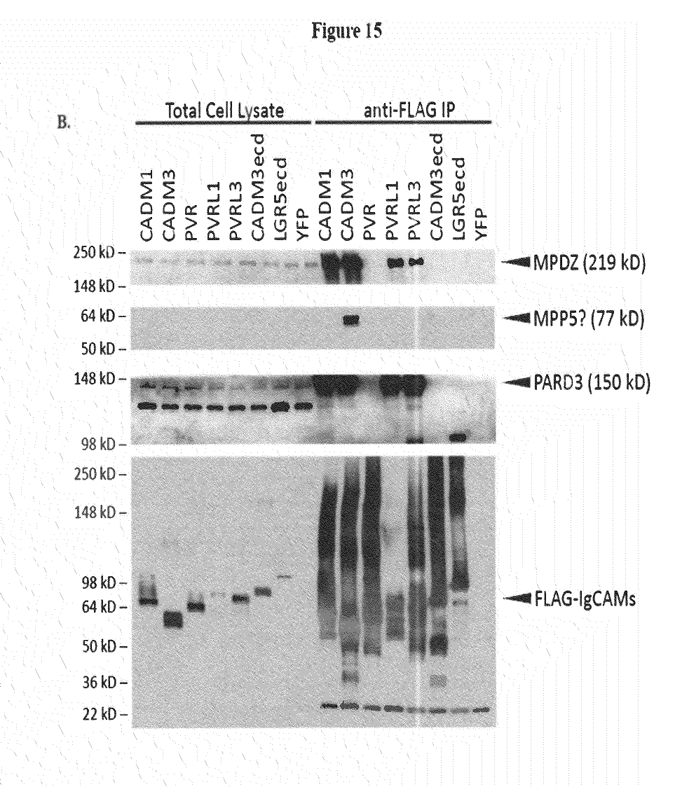

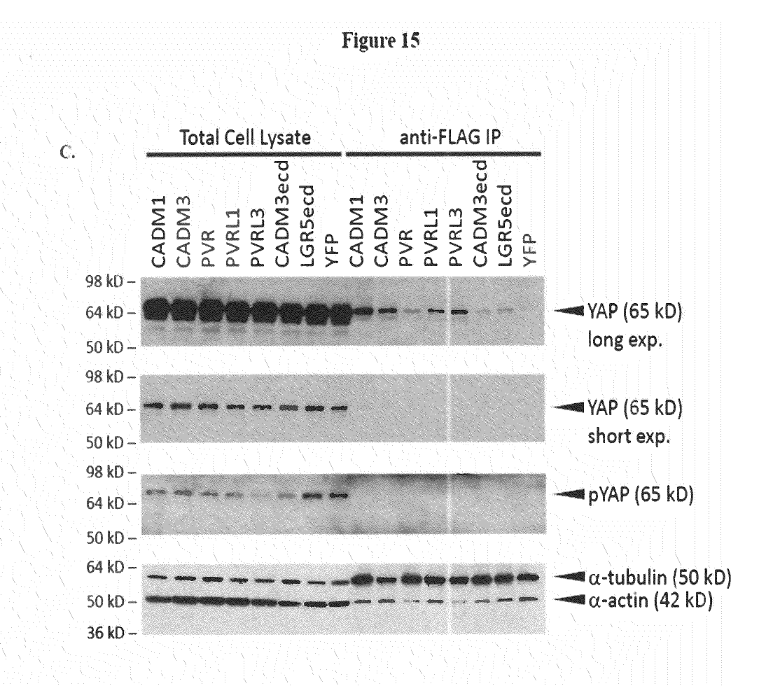

To validate these protein complexes, immunoprecipitates (see method described above) were prepared from HEK-293T cells transiently transfected with FLAG-tagged full-length CADM1, CADM3, PVR, PVRL1, and PVRL3, with CADM3-CD4TM-GFP and LGR5-CD4TM-GFP constructs as internal negative controls. Total cell lysates and the immunoprecipitate eluates were run on 4-12% Tris-Glycine Midi Gels (Life Technologies/Invitrogen, Grand Island, N.Y.) and transferred by iBlot onto PVDF membranes (Life Technologies/Invitrogen). Blots were probed with antibodies against the following proteins: Afadin/MLLT4 (#6492; Cell Signaling Technology, Danvers, Mass.); DCAF7 (#ab138490; Abcam, Cambridge, Mass.); DLG1 (#ab134156); INADL (#ab151257; Abcam); LIN7 (Abcam, ab127049; Abcam); LIN7C (#ab82646; Abcam); MPDZ (#ab101277; Abcam); MPP5 (#ab155132; Abcam); PARD3 (#ab64646; Abcam); YAP1 (#ab52771; Abcam); phospho-YAP1 (#4911; Cell Signaling Technology), and tubulin and actin as controls. Western blots are shown in FIGS. 15A-15C and the positively-identified complexes containing IgCAMs are summarized in Table 5.

TABLE 5

| | CADM1 | CADM3 | PVR | PVRL1 | PVRL3 |
|---|---|---|---|---|---|
| Afadin | − | − | − | + | ++ |
| DCAF7 | +++ | ++++ | + | + | ++ |
| DLG1 | +++ | ++++ | − | ++ | + |
| INADL | − | + | − | − | − |
| LIN7 | +++ | +++ | + | ++ | ++ |
| LIN7C | − | + | − | − | ++ |
| MPDZ | +++ | +++ | − | ++ | + |
| MPP5 | − | + | − | − | − |
| PARD3 | ++ | + | − | + | ++ |
| YAP1 | +++ | ++ | − | + | ++ |

Of particular note, YAP1 co-immunoprecipitated with CADM1, CADM3, PVRL1, and PVRL3 supporting the hypothesis that IgCAMs are involved in the Hippo pathway.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

The sequences disclosed in this application are as follows:

JAM Family
Human AMICA with predicted signal sequence underlined
(SEQ ID NO: 1)
MECPLKLILLPVLLDYSLGLNDLNVSPPELTVHVGDSALMGCVFQSTEDKCIFKIDWTLS
PGEHAKDEYVLYYYSNLSVPIGRFQNRVHLMGDILCNDGSLLLQDVQEADQGTYICEIRL
KGESQVFKKAVVLHVLPEEPKELMVHVGGLIQMGCVFQSTEVKHVTKVEWIFSGRRAKEE
IVERYYHKLRMSVEYSQSWGHFQNRVNLVGDIFRNDGSIMLQGVRESDGGNYTCSIHLGN
LVFKKTIVLHVSPEEPRTLVTPAALRPLVLGGNQLVIIVGIVCATILLLPVLILIVKKTC
GNKSSVNSTVLVKNTKKTNPEIKEKPCHFERCEGEKHIYSPIIVREVIEEEEPSEKSEAT
YMTMHPVWPSLRSDRNNSLEKKSGGGMPKTQQAF
Human CAR with predicted signal sequence underlined
(SEQ ID NO: 2)
MALLLCFVLLCGVVDFARSLSITTPEEMIEKAKGETAYLPCKFTLSPEDQGPLDIEWLIS
PADNQKVDQVIILYSGDKIYDDYYPDLKGRVHFTSNDLKSGDASINVTNLQLSDIGTYQC
KVKKAPGVANKKIHLVVLVKPSGARCYVDGSEEIGSDFKIKCEPKEGSLPLQYEWQKLSD
SQKMPTSWLAEMTSSVISVKNASSEYSGTYSCTVRNRVGSDQCLLRLNVVPPSNKAGLIA
GAIIGTLLALALIGLIIFCCRKKRREEKYEKEVHHDIREDVPPPKSRTSTARSYIGSNHS
SLGSMSPSNMEGYSKTQYNQVPSEDFERTPQSPTLPPAKFKYAY
Human CLMP with predicted signal sequence underlined
(SEQ ID NO: 3)
MSLLLLLLLVSYYVGTLGTHTEIKRVAEEKVTLPCHHQLGLPEKDTLDIEWLLTDNEGNQ
KVVITYSSRHVYNNLTEEQKGRVAFASNFLAGDASLQIEPLKPSDEGRYTCKVKNSGRYV
WSHVILKVLVRPSKPKCELEGELTEGSDLTLQCESSSGTEPIVYYWQRIREKEGEDERLP
PKSRIDYNHPGRVLLQNLTMSYSGLYQCTAGNEAGKESCVVRVTVQYVQSIGMVAGAVTG
IVAGALLIFLLVWLLIRRKDKERYEEEERPNEIREDAEAPKARLVKPSSSSSGSRSSRSG
SSSTRSTANSASRSQRTLSTDAAPQPGLATQAYSLVGPEVRGSEPKKVHHANLTKAETTP
SMIPSQSRAFQTV
Human ESAM with predicted signal sequence underlined
(SEQ ID NO: 4)
MISLPGPLVTNLLRFLFLGLSALAPPSRAQLQLHLPANRLQAVEGGEVVLPAWYTLHGEV
SSSQPWEVPFVMWFFKQKEKEDQVLSYINGVTTSKPGVSLVYSMPSRNLSLRLEGLQEKD
SGPYSCSVNVQDKQGKSRGHSIKTLELNVLVPPAPPSCRLQGVPHVGANVTLSCQSPRSK
PAVQYQWDRQLPSFQTFFAPALDVIRGSLSLTNLSSSMAGVYVCKAHNEVGTAQCNVTLE
VSTGPGAAVVAGAVVGTLVGLGLLAGLVLLYHRRGKALEEPANDIKEDAIAPRTLPWPKS
SDTISKNGTLSSVTSARALRPPHGPPRPGALTPTPSLSSQALPSPRLPTTDGAHPQPISP
IPGGVSSSGLSRMGAVPVMVPAQSQAGSLV
Human GP A33 with predicted signal sequence underlined
(SEQ ID NO: 5)
MVGKMWPVLWTLCAVRVTVDAISVETPQDVLRASQGKSVTLPCTYHTSTSSREGLIQWDK
LLLTHTERVVIWPFSNKNYIHGELYKNRVSISNNAEQSDASITIDQLTMADNGTYECSVS
LMSDLEGNTKSRVRLLVLVPPSKPECGIEGETIIGNNIQLTCQSKEGSPTPQYSWNRYNI
LNQEQPLAQPASGQPVSLKNISTDTSGYYICTSSNEEGTQFCNITVAVRSPSMNVALYVG
IAVGVVAALIIIGIIIYCCCCRGKDDNTEDKEDARPNREAYEEPPEQLRELSREREEEDD
YRQEEQRSTGRESPDHLDQ
Human JAM1 with predicted signal sequence underlined
(SEQ ID NO: 6)
MGTKAQVERKLLCLFILAILLCSLALGSVTVHSSEPEVRIPENNPVKLSCAYSGFSSPRV
EWKFDQGDTTRLVCYNNKITASYEDRVTFLPTGITFKSVTREDTGTYTCMVSEEGGNSYG
EVKVKLIVLVPPSKPTVNIPSSATIGNRAVLTCSEQDGSPPSEYTWFNDGIVMPTNPKST
RAFSNSSYVLNPTTGELVFDPLSASIDTGEYSCEARNGYGTPMTSNAVRMEAVERNVGIV
AAVLVTITLLGILVFGIWFAYSRGHFDRTKKGTSSKKVIYSQPSARSEGEFKQTSSFLV
Human JAM2 with predicted signal sequence underlined
(SEQ ID NO: 7)
MARRSRHRLLLLLLRYLVVALGYHKAYGFSAPKDQQVVTAVEYQEAILACKTPKKTVSSR
LEWKKLGRSVSFVYYQQTLQGDFKNRAEMIDFNIRIKNVTRSDAGKYRCEVSAPSEQGQN
LEEDTVTLEVLVAPAVPSCEVPSSALSGTVVELRCQDKEGNPAPEYTWFKDGIRLLENPR
LGSQSTNSSYTMNTKTGTLQFNTVSKLDTGEYSCEARNSVGYRRCPGKRMQVDDLNISGI
IAAVVVVALVISVCGLGVCYAQRKGYFSKETSFQKSNSSSKATTMSENDFKHTKSFII
Human JAM3 with predicted signal sequence underlined
(SEQ ID NO: 8)
MALRRPPRLRLCARLPDFFLLLLFRGCLIGAVNLKSSNRTPVVQEFESVELSCIITDSQT
SDPRIEWKKIQDEQTTYVFFDNKIQGDLAGRAEILGKTSLKIWNVTRRDSALYRCEVVAR
NDRKEIDEIVIELTVQVKPVTPVCRVPKAVPVGKMATLHCQESEGHPRPHYSWYRNDVPL
PTDSRANPRFRNSSFHLNSETGTLVFTAVHKDDSGQYYCIASNDAGSARCEEQEMEVYDL
NIGGIIGGVLVVLAVLALITLGICCAYRRGYFINNKQDGESYKNPGKPDGVNYIRTDEEG
DFRHKSSFVI
Human VSIG1 with predicted signal sequence underlined
(SEQ ID NO: 9)
MVFAFWKVFLILSCLAGQVSVVQVTIPDGFVNVTVGSNVTLICIYTTTVASREQLSIQWS
FFHKKEMEPISHSSCLSTEGMEEFAVSQCLKMTHARDARGRCSWTSEIYFSQGGQAVAIG
QFKDRITGSNDPGNASITISHMQPADSGIYICDVNNPPDFLGQNQGILNVSVLVKPSKPL
CSVQGRPETGHTISLSCLSALGTPSPVYYWHKLEGRDIVPVKENFNPTTGILVIGNLTNF
EQGYYQCTAINRLGNSSCEIDLTSSHPEVGIIVGALIGSLVGAAIIISVVCFARNKAKAK
AKERNSKTIAELEPMTKINPRGESEAMPREDATQLEVTLPSSIHETGPDTIQEPDYEPKP
TQEPAPEPAPGSEPMAVPDLDIELELEPETQSELEPEPEPEPESEPGVVVEPLSEDEKGV
VKA
Human VSIG2 with predicted signal sequence underlined
(SEQ ID NO: 10)
MAELPGPFLCGALLGFLCLSGLAVEVKVPTEPLSTPLGKTAELTCTYSTSVGDSFALEWS
FVQPGKPISESHPILYFTNGHLYPTGSKSKRVSLLQNPPTVGVATLKLTDVHPSDTGTYL
CQVNNPPDFYTNGLGLINLTVLVPPSNPLCSQSGQTSVGGSTALRCSSSEGAPKPVYNWV
RLGTFPTPSPGSMVQDEVSGQLILTNLSLTSSGTYRCVATNQMGSASCELTLSVTEPSQG
RVAGALIGVLLGVLLLSVAAFCLVRFQKERGKKPKETYGGSDLREDAIAPGISEHTCMRA
DSSKGFLERPSSASTVTTTKSKLPMVV
Human VSIG3 with predicted signal sequence underlined
(SEQ ID NO: 11)
MSLVELLLWWNCFSRTGVAASLEVSESPGSIQVARGQPAVLPCTFTTSAALINLNVIWMV
TPLSNANQPEQVILYQGGQMFDGAPRFHGRVGFTGTMPATNVSIFINNTQLSDTGTYQCL
VNNLPDIGGRNIGVTGLTVLVPPSAPHCQIQGSQDIGSDVILLCSSEEGIPRPTYLWEKL
DNTLKLPPTATQDQVQGTVTIRNISALSSGLYQCVASNAIGTSTCLLDLQVISPQPRNIG
LIAGAIGTGAVIIIFCIALILGAFFYWRSKNKEEEEEEIPNEIREDDLPPKCSSAKAFHT
EISSSDNNTLTSSNAYNSRYWSNNPKVHRNTESVSHFSDLGQSFSFHSGNANIPSIYANG
THLVPGQHKTLVVTANRGSSPQVMSRSNGSVSRKPRPPHTHSYTISHATLERIGAVPVMV
PAQSRAGSLV
Human VSIG4 with predicted signal sequence underlined
(SEQ ID NO: 12)
MGILLGLLLLGHLTVDTYGRPILEVPESVTGPWKGDVNLPCTYDPLQGYTQVLVKWLVQR
GSDPVTIFLRDSSGDHIQQAKYQGRLHVSHKVPGDVSLQLSTLEMDDRSHYTCEVTWQTP
DGNQVVRDKITELRVQKLSVSKPTVTTGSGYGFTVPQGMRISLQCQARGSPPISYIWYKQ
QTNNQEPIKVATLSTLLFKPAVIADSGSYFCTAKGQVGSEQHSDIVKFVVKDSSKLLKTK
TEAPTTMTYPLKATSTVKQSWDWTTDMDGYLGETSAGPGKSLPVFAIILIISLCCMVVFT
MAYIMLCRKTSQQEHVYEAARAHAREANDSGETMRVAIFASGCSSDEPTSQNLGNNYSDE
PCIGQEYQIIAQINGNYARLLDTVPLDYEFLATEGKSVC
Human VSIG8 with predicted signal sequence underlined
(SEQ ID NO: 13)
MRVGGAFHLLLVCLSPALLSAVRINGDGQEVLYLAEGDNVRLGCPYVLDPEDYGPNGLDI
EQMQVNSDPAHHRENVFLSYQDKRINHGSLPHLQQRVRFAASDPSQYDASINLMNLQVSD
TATYECRVKKTTMATRKVIVTVQARPAVPMCWTEGHMTYGNDVVLKCYASGGSQPLSYKW
AKISGHHYPYRAGSYTSQHSYHSELSYQESFHSSINQGLNNGDLVLKDISRADDGLYQCT
VANNVGYSVCVVEVKVSDSRRIGVIIGIVLGSLLALGCLAVGIWGLVCCCCGGSGAGGAR
GAFGYGNGGGVGGGACGDLASEIREDAVAPGCKASGRGSRVTHLLGYPTQNVSRSLRRKY
APPPCGGPEDVALAPCTAAAACEAGPSPVYVKVKSAEPADCAEGPVQCKNGLLV
CADM Family
Human CADM1 with predicted signal sequence underlined
(SEQ ID NO: 14)
MASVVLPSGSQCAAAAAAAAPPGLRLRLLLLLFSAAALIPTGDGQNLFTKDVTVIEGEVA
TISCQVNKSDDSVIQLLNPNRQTIVEREFRPLKDSRFQLLNFSSSELKVSLTNVSISDEG
RYFCQLYTDPPQESYTTITVLVPPRNLMIDIQKDTAVEGEEIEVNCTAMASKPATTIRWF
KGNTELKGKSEVEEWSDMYTVTSQLMLKVHKEDDGVPVICQVEHPAVTGNLQTQRYLEVQ
YKPQVHIQMTYPLQGLTREGDALELTCEAIGKPQPVMVTWVRVDDEMPQHAVLSGPNLFI
NNLNKTDNGTYRCEASNIVGKAHSDYMLYVYDPPTTIPPPTTTTTTTTTTTTTILTIITD
SRAGEEGSIRAVDHAVIGGVVAVVVFAMLCLLIILGRYFARHKGTYFTHEAKGADDAADA
DTAIINAEGGQNNSEEKKEYFI
Human CADM2 with predicted signal sequence underlined
(SEQ ID NO: 15)
MIWKRSAVLRFYSVCGLLLQGSQGQFPLTQNVTVVEGGTAILTCRVDQNDNTSLQWSNPA
QQTLYFDDKKALRDNRIELVRASWHELSISVSDVSLSDEGQYTCSLFTMPVKTSKAYLTV
LGVPEKPQISGFSSPVMEGDLMQLTCKTSGSKPAADIRWFKNDKEIKDVKYLKEEDANRK
TFTVSSTLDFRVDRSDDGVAVICRVDHESLNATPQVAMQVLEIHYTPSVKIIPSTPFPQE
GQPLILTCESKGKPLPEPVLWTKDGGELPDPDRMVVSGRELNILFLNKTDNGTYRCEATN
TIGQSSAEYVLIVHDVPNTLLPTTIIPSLTTATVTTTVAITTSPTTSATTSSIRDPNALA
GQNGPDHALIGGIVAVVVFVTLCSIFLLGRYLARHKGTYLTNEAKGAEDAPDADTAIINA
EGSQVNAEEKKEYFI
Human CADM3 with predicted signal sequence underlined
(SEQ ID NO: 16)
MGAPAASLLLLLLLFACCWAPGGANLSQDGYWQEQDLELGTLAPLDEAISSTVWSSPDML
ASQDSQPWTSDETVVAGGTVVLKCQVKDHEDSSLQWSNPAQQTLYFGEKRALRDNRIQLV
TSTPHELSISISNVALADEGEYTCSIFTMPVRTAKSLVTVLGIPQKPIITGYKSSLREKD
TATLNCQSSGSKPAARLTWRKGDQELHGEPTRIQEDPNGKTFTVSSSVTFQVTREDDGAS
IVCSVNHESLKGADRSTSQRIEVLYTPTAMIRPDPPHPREGQKLLLHCEGRGNPVPQQYL
WEKEGSVPPLKMTQESALIFPFLNKSDSGTYGCTATSNMGSYKAYYTLNVNDPSPVPSSS
STYHAIIGGIVAFIVFLLLIMLIFLGHYLIRHKGTYLTHEAKGSDDAPDADTAIINAEGG
QSGGDDKKEYFI
Human CADM4 with predicted signal sequence underlined
(SEQ ID NO: 17)
MGRARRFQWPLLLLWAAAAGPGAGQEVQTENVTVAEGGVAEITCRLHQYDGSIVVIQNPA
RQTLFFNGTRALKDERFQLEEFSPRRVRIRLSDARLEDEGGYFCQLYTEDTHHQIATLTV
LVAPENPVVEVREQAVEGGEVELSCLVPRSRPAATLRWYRDRKELKGVSSSQENGKVWSV
ASTVRFRVDRKDDGGIIICEAQNQALPSGHSKQTQYVLDVQYSPTARIHASQAVVREGDT
LVLTCAVTGNPRPNQIRWNRGNESLPERAEAVGETLTLPGLVSADNGTYTCEASNKHGHA
RALYVLVVYDPGAVVEAQTSVPYAIVGGILALLVFLIICVLVGMVWCSVRQKGSYLTHEA
SGLDEQGEAREAFLNGSDGHKRKEEFFI
Human CRTAM with predicted signal sequence underlined
(SEQ ID NO: 18)
MWWRVLSLLAWFPLQEASLTNHTETITVEEGQTLTLKCVTSLRKNSSLQWLTPSGFTIFL
NEYPALKNSKYQLLHHSANQLSITVPNVTLQDEGVYKCLHYSDSVSTKEVKVIVLATPFK
PILEASVIRKQNGEEHVVLMCSTMRSKPPPQITWLLGNSMEVSGGTLHEFETDGKKCNTT
STLIIHTYGKNSTVDCIIRHRGLQGRKLVAPFRFEDLVTDEETASDALERNSLSSQDPQQ
PTSTVSVTEDSSTSEIDKEEKEQTTQDPDLTTEANPQYLGLARKKSGILLLTLVSFLIFI
LFIIVQLFIMKLRKAHVIWKKENEVSEHTLESYRSRSNNEETSSEEKNGQSSHPMRCMNY
ITKLYSEAKTKRKENVQHSKLEEKHIQVPESIV
Human TMIGD1 with predicted signal sequence underlined
(SEQ ID NO: 19)
MAWKSSVIMQMGRFLLLVILFLPREMTSSVLTVNGKTENYILDTTPGSQASLICAVQNHT
REEELLWYREEGRVDLKSGNKINSSSVCVSSISENDNGISFTCRLGRDQSVSVSVVLNVT
FPPLLSGNDFQTVEEGSNVKLVCNVKANPQAQMMWYKNSSLLDLEKSRHQIQQTSESFQL
SITKVEKPDNGTYSCIAKSSLKTESLDFHLIVKDKTVGVPIEPIIAACVVIFLTLCFGLI
ARRKKIMKLCMKDKDPHSETAL
PVRFamily
Human CD96 with predicted signal sequence underlined
(SEQ ID NO: 20)
MEKKWKYCAVYYIIQIHFVKGVWEKTVNTEENVYATLGSDVNLTCQTQTVGFFVQMQWSK
VTNKIDLIAVYHPQYGFYCAYGRPCESLVTFTETPENGSKWTLHLRNMSCSVSGRYECML
VLYPEGIQTKIYNLLIQTHVTADEWNSNHTIEIEINQTLEIPCFQNSSSKISSEFTYAWS
VENSSTDSWVLLSKGIKEDNGTQETLISQNHLISNSTLLKDRVKLGTDYRLHLSPVQIFD
DGRKFSCHIRVGPNKILRSSTTVKVFAKPEIPVIVENNSTDVLVERRFTCLLKNVFPKAN
ITWFIDGSFLHDEKEGIYITNEERKGKDGFLELKSVLTRVHSNKPAQSDNLTIWCMALSP
VPGNKVWNISSEKITFLLGSEISSTDPPLSVTESTLDTQPSPASSVSPARYPATSSVTLV
DVSALRPNTTPQPSNSSMTTRGFNYPWTSSGTDTKKSVSRIPSETYSSSPSGAGSTLHDN
VFTSTARAFSEVPTTANGSTKTNHVHITGIVVNKPKDGMSWPVIVAALLFCCMILFGLGV
RKWCQYQKEIMERPPPFKPPPPPIKYTCIQEPNESDLPYHEMETL
Human CD200 with predicted signal sequence underlined
(SEQ ID NO: 21)
MERLVIRMPFSHLSTYSLVWVMAAVVLCTAQVQVVTQDEREQLYTPASLKCSLQNAQEAL
IVTWQKKKAVSPENMVTFSENHGVVIQPAYKDKINITQLGLQNSTITFWNITLEDEGCYM
CLFNTPGFGKISGTACLTVYVQPIVSLHYKFSEDHLNITCSATARPAPMVFWKVPRSGIE
NSTVTLSHPNGTTSVTSILHIKDPKNQVGKEVICQVLHLGTVTDFKQTVNKGYWFSVPLL
LSIVSLVILLVLISILLYWKRHRNQDREP
Human CD200R1 with predicted signal sequence underlined
(SEQ ID NO: 22)
MLCPWRTANLGLLLILTIFLVAEAEGAAQPNNSLMLQTSKENHALASSSLCMDEKQITQN
YSKVLAEVNTSWPVKMATNAVLCCPPIALRNLIIITWEIILRGQPSCTKAYKKETNETKE
TNCTDERITWVSRPDQNSDLQIRTVAITHDGYYRCIMVTPDGNFHRGYHLQVLVTPEVTL
FQNRNRTAVCKAVAGKPAAHISWIPEGDCATKQEYWSNGTVTVKSTCHWEVHNVSTVTCH
VSHLTGNKSLYIELLPVPGAKKSAKLYIPYIILTIIILTIVGFIWLLKVNGCRKYKLNKT
ESTPVVEEDEMQPYASYTEKNNPLYDTTNKVKASEALQSEVDTDLHTL
Human CD200R1L with predicted signal sequence underlined
(SEQ ID NO: 23)
MSAPRLLISIIIMVSASSSSCMGGKQMTQNYSTIFAEGNISQPVLMDINAVLCCPPIALR
NLIIITWEIILRGQPSCTKAYKKETNETKETNCTVERITWVSRPDQNSDLQIRPVDTTHD
GYYRGIVVTPDGNFHRGYHLQVLVTPEVNLFQSRNITAVCKAVTGKPAAQISWIPEGSIL
ATKQEYWGNGTVTVKSTCPWEGHKSTVTCHVSHLTGNKSLSVKLNSGLRTSGSPALSLLI
ILYVKLSLFVVILVTTGFVFFQRINHVRKVL
Human CD226 with predicted signal sequence underlined
(SEQ ID NO: 24)
MDYPTLLLALLHVYRALCEEVLWHTSVPFAENMSLECVYPSMGILTQVEWFKIGTQQDSI
AIFSPTHGMVIRKPYAERVYFLNSTMASNNMTLFFRNASEDDVGYYSCSLYTYPQGTWQK
VIQVVQSDSFEAAVPSNSHIVSEPGKNVTLTCQPQMTWPVQAVRWEKIQPRQIDLLTYCN
LVHGRNFTSKFPRQIVSNCSHGRWSVIVIPDVTVSDSGLYRCYLQASAGENETFVMRLTV
AEGKTDNQYTLFVAGGTVLLLLFVISITTIIVIFLNRRRRRERRDLFTESWDTQKAPNNY
RSPISTSQPTNQSMDDTREDIYVNYPTFSRRPKTRV
Human PVRIG with predicted signal sequence underlined
(SEQ ID NO: 25)
MRTEAQVPALQPPEPGLEGAMGHRTLVLPWVLLTLCVTAGTPEVWVQVRMEATELSEFTI
RCGFLGSGSISLVTVSWGGPNGAGGTTLAVLHPERGIRQWAPARQARWETQSSISLILEG
SGASSPCANTTFCCKFASFPEGSWEACGSLPPSSDPGLSAPPTPAPILRADLAGILGVSG
VLLFGCVYLLHLLRRHKHRPAPRLQPSRTSPQAPRARAWAPSQASQAALHVPYATINTSC
RPATLDTAHPHGGPSWWASLPTHAAHRPQGPAAWASTPIPARGSFVSVENGLYAQAGERP
PHTGPGLTLFPDPRGPRAMEGPLGVR
Human PVR with predicted signal sequence underlined
(SEQ ID NO: 26)
MARAMAAAWPLLLVALLVLSWPPPGTGDVVVQAPTQVPGFLGDSVTLPCYLQVPNMEVTH
VSQLTWARHGESGSMAVFHQTQGPSYSESKRLEFVAARLGAELRNASLRMFGLRVEDEGN
YTCLFVTFPQGSRSVDIWLRVLAKPQNTAEVQKVQLTGEPVPMARCVSTGGRPPAQITWH
SDLGGMPNTSQVPGFLSGTVTVTSLWILVPSSQVDGKNVTCKVEHESFEKPQLLTVNLTV
YYPPEVSISGYDNNWYLGQNEATLTCDARSNPEPTGYNWSTTMGPLPPFAVAQGAQLLIR
PVDKPINTTLICNVTNALGARQAELTVQVKEGPPSEHSGMSRNAIIFLVLGILVFLILLG
IGIYFYWSKCSREVLWHCHLCPSSTEHASASANGHVSYSAVSRENSSSQDPQTEGTR
Human PVRL1 with predicted signal sequence underlined
(SEQ ID NO: 27)
MARMGLAGAAGRWWGLALGLTAFFLPGVHSQVVQVNDSMYGFIGTDVVLHCSFANPLPSV
KITQVTWQKSTNGSKQNVAIYNPSMGVSVLAPYRERVEFLRPSFTDGTIRLSRLELEDEG
VYICEFATFPTGNRESQLNLTVMAKPTNWIEGTQAVLRAKKGQDDKVLVATCTSANGKPP
SVVSWETRLKGEAEYQEIRNPNGTVTVISRYRLVPSREAHQQSLACIVNYHMDRFKESLT
LNVQYEPEVTIEGFDGNWYLQRMDVKLTCKADANPPATEYHWTTLNGSLPKGVEAQNRTL
FFKGPINYSLAGTYICEATNPIGTRSGQVEVNITEFPYTPSPPEHGRRAGPVPTAIIGGV
AGSILLVLIVVGGIVVALRRRRHTFKGDYSTKKHVYGNGYSKAGIPQHHPPMAQNLQYPD
DSDDEKKAGPLGGSSYEEEEEEEEGGGGGERKVGGPHPKYDEDAKRPYFTVDEAEARQDG
YGDRTLGYQYDPEQLDLAENMVSQNDGSFISKKEWYV
Human PVRL2 with predicted signal sequence underlined
(SEQ ID NO: 28)
MARAAALLPSRSPPTPLLWPLLLLLLLETGAQDVRVQVLPEVRGQLGGTVELPCHLLPPV
PGLYISLVTWQRPDAPANHQRVAAFHPKMGPSFPSPKPGSERLSFVSAKQSTGQDTEAEL
QDATLALHGLTVEDEGNYTCEFATFPKGSVRGMTWLRVIAKPKNQAEAQKVTFSQDPTTV
ALCISKEGRPPARISWLSSLDWEAKETQVSGTLAGTVTVTSRFTLVPSGRADGVTVTCKV
EHESFEEPALIPVTLSVRYPPEVSISGYDDNWYLGRTDATLSCDVRSNPEPTGYDWSTTS
GTFPTSAVAQGSQLVIHAVDSLFNTTFVCTVTNAVGMGRAEQVIFVRETPNTAGAGATGG
IIGGIIAAIIATAVAATGILICRQQRKEQTLQGAEEDEDLEGPPSYKPPTPKAKLEAQEM
PSQLFTLGASEHSPLKTPYFDAGASCTEQEMPRYHELPTLEERSGPLHPGATSLGSPIPV
PPGPPAVEDVSLDLEDEEGEEEEEYLDKINPIYDALSYSSPSDSYQGKGFVMSRAMYV
Human PVRL3 with predicted signal sequence underlined
(SEQ ID NO: 29)
MARTLRPSPLCPGGGKAQLSSASLLGAGLLLQPPTPPPLLLLLFPLLLFSRLCGALAGPI
IVEPHVTAVWGKNVSLKCLIEVNETITQISWEKIHGKSSQTVAVHHPQYGFSVQGEYQGR
VLFKNYSLNDATITLHNIGFSDSGKYICKAVTFPLGNAQSSTTVTVLVEPTVSLIKGPDS
LIDGGNETVAAICIAATGKPVAHIDWEGDLGEMESTTTSFPNETATIISQYKLEPTRFAR
GRRITCVVKHPALEKDIRYSFILDIQYAPEVSVTGYDGNWFVGRKGVNLKCNADANPPPF
KSVWSRLDGQWPDGLLASDNTLHFVHPLTFNYSGVYICKVTNSLGQRSDQKVIYISDPPT
TTTLQPTIQWHPSTADIEDLATEPKKLPFPLSTLATIKDDTIATIIASVVGGALFIVLVS
VLAGIFCYRRRRTFRGDYFAKNYIPPSDMQKESQIDVLQQDELDSYPDSVKKENKNPVNN
LIRKDYLEEPEKTQWNNVENLNRFERPMDYYEDLKMGMKFVSDEHYDENEDDLVSHVDGS
VISRREWYV
Human PVRL4 with predicted signal sequence underlined
(SEQ ID NO: 30)
MPLSLGAEMWGPEAWLLLLLLLASFTGRCPAGELETSDVVTVVLGQDAKLPCFYRGDSGE
QVGQVAWARVDAGEGAQELALLHSKYGLHVSPAYEGRVEQPPPPRNPLDGSVLLRNAVQA
DEGEYECRVSTFPAGSFQARLRLRVLVPPLPSLNPGPALEEGQGLTLAASCTAEGSPAPS
VTWDTEVKGTTSSRSFKHSRSAAVTSEFHLVPSRSMNGQPLTCVVSHPGLLQDQRITHIL
HVSFLAEASVRGLEDQNLWHIGREGAMLKCLSEGQPPPSYNWTRLDGPLPSGVRVDGDTL
GFPPLTTEHSGIYVCHVSNEFSSRDSQVTVDVLDPQEDSGKQVDLVSASVVVVGVIAALL
FCLLVVVVVLMSRYHRRKAQQMTQKYEEELTLTRENSIRRLHSHHTDPRSQPEESVGLRA
EGHPDSLKDNSSCSVMSEEPEGRSYSTLTTVREIETQTELLSPGSGRAEEEEDQDEGIKQ
AMNHFVQENGTLRAKPTGNGIYINGRGHLV
Human TIGIT with predicted signal sequence underlined
(SEQ ID NO: 31)
MRWCLLLIWAQGLRQAPLASGMMTGTIETTGNISAEKGGSIILQCHLSSTTAQVTQVNWE
QQDQLLAICNADLGWHISPSFKDRVAPGPGLGLTLQSLTVNDTGEYFCIYHTYPDGTYTG
RIFLEVLESSVAEHGARFQIPLLGAMAATLVVICTAVIVVVALTRKKKALRIHSVEGDLR
RKSAGQEEWSPSAPSPPGSCVQAEAAPAGLCGEQRGEDCAELHDYFNVLSYRSLGNCSFF
TETG
Human TMEM25 with predicted signal sequence underlined
(SEQ ID NO: 32)
MALPPGPAALRHTLLLLPALLSSGWGELEPQIDGQTWAERALRENERHAFTCRVAGGPGT
PRLAWYLDGQLQEASTSRLLSVGGEAFSGGTSTFTVTAHRAQHELNCSLQDPRSGRSANA
SVILNVQFKPEIAQVGAKYQEAQGPGLLVVLFALVRANPPANVTWIDQDGPVTVNTSDFL
VLDAQNYPWLTNHTVQLQLRSLAHNLSVVATNDVGVTSASLPAPGLLATRVEVPLLGIVV
AAGLALGTLVGFSTLVACLVCRKEKKTKGPSRHPSLISSDSNNLKLNNVRLPRENMSLPS
NLQLNDLTPDSRAVKPADRQMAQNNSRPELLDPEPGGLLTSQGFIRLPVLGYIYRVSSVS
SDEIWL
JAM Family
Human AMICA - ECD without predicted signal sequence
(SEQ ID NO: 33)
LNDLNVSPPELTVHVGDSALMGCVFQSTEDKCIFKIDWTLSPGFHAKDEYVLYYYSNLSV
PIGRFQNRVHLMGDILCNDGSLLLQDVQEADQGTYICEIRLKGESQVFKKAVVLHVLPEE
PKELMVHVGGLIQMGCVFQSTEVKHVTKVEWIFSGRRAKEEIVFRYYHKLRMSVEYSQSW
GHFQNRVNLVGDIFRNDGSIMLQGVRESDGGNYTCSIHLGNLVFKKTIVLHVSPEEPRTL
VTPAALRPLVLGGNQLV
Human CAR - ECD without predicted signal sequence
(SEQ ID NO: 34)
LSITTPEEMIEKAKGETAYLPCKFTLSPEDQGPLDIEWLISPADNQKVDQVIILYSGDKI
YDDYYPDLKGRVHFTSNDLKSGDASINVTNLQLSDIGTYQCKVKKAPGVANKKIHLVVLV
KPSGARCYVDGSEEIGSDFKIKCEPKEGSLPLQYEWQKLSDSQKMPTSWLAEMTSSVISV
KNASSEYSGTYSCTVRNRVGSDQCLLRLNVVPPSNKAG
Human CLMP - ECD without predicted signal sequence
(SEQ ID NO: 35)
THTEIKRVAEEKVTLPCHHQLGLPEKDTLDIEWLLTDNEGNQKVVITYSSRHVYNNLTEE
QKGRVAFASNFLAGDASLQIEPLKPSDEGRYTCKVKNSGRYVWSHVILKVLVRPSKPKCE
LEGELTEGSDLTLQCESSSGTEPIVYYWQRIREKEGEDERLPPKSRIDYNHPGRVLLQNL
TMSYSGLYQCTAGNEAGKESCVVRVTVQYVQSIGMVA
Human ESAM - ECD without predicted signal sequence
(SEQ ID NO: 36)
QLQLHLPANRLQAVEGGEVVLPAWYTLHGEVSSSQPWEVPFVMWFFKQKEKEDQVLSYIN
GVTTSKPGVSLVYSMPSRNLSLRLEGLQEKDSGPYSCSVNVQDKQGKSRGHSIKTLELNV
LVPPAPPSCRLQGVPHVGANVTLSCQSPRSKPAVQYQWDRQLPSFQTFFAPALDVIRGSL
SLTNLSSSMAGVYVCKAHNEVGTAQCNVTLEVSTGPGAA
Human GP A33 - ECD without predicted signal sequence
(SEQ ID NO: 37)
ISVSTPQDVLRASQGKSVTLPCTYHTSTSSREGLIQWDKLLLTHTERVVIWPFSNKNYIH
GELYKNRVSISNNAEQSDASITIDQLTMADNGTYECSVSLMSDLEGNTKSRVRLLVLVPP
SKPECGIEGETIIGNNIQLTCQSKEGSPTPQYSWKRYNILNQEQPLAQPASGQPVSLKNI
STDTSGYYICTSSNEEGTQFCNITVAVRSPSMNV
Human JAM1 - ECD without predicted signal sequence
(SEQ ID NO: 38)
SVTVHSSEPEVRIPENNPVKLSCAYSGFSSPRVEWKFDQGDTTRLVCYNNKITASYEDRV
TFLPTGITFKSVTREDTGTYTCMVSEKGGNSYGEVKVKLIVLVPPSKPTVNIPSSATIGN
RAVLTCSEQDGSPPSEYTWFKDGIVMPTNPKSTRAFSNSSYVLNPTTGELVFDPLSASDT
GEYSCEARNGYGTPMTSNAVRMEAVERNVGV
Human JAM2 - ECD without predicted signal sequence
(SEQ ID NO: 39)
FSAPKDQQVVTAVEYQEAILACKTPKKTVSSRLEWKKLGRSVSFVYYQQTLQGDFKNRAE
MIDFNTRIKNVTRSDAGKYRCEVSAPSEQGQNLEEDTVTLEVLVAPAVPSCEVPSSALSG
TVVELRCQDKEGNPAPEYTWFKDGIRLLENPRLGSQSTNSSYTMNTKTGTLQFNTVSKLD
TGEYSCEARNSVGYRRCPGKRMQVDDLNIS
Human JAMS - ECD without predicted signal sequence
(SEQ ID NO: 40)
VNLKSSNRTPVVQEFESVELSCIITDSQTSDPRIEWKKIQDEQTTYVFFDNKIQGDLAGR
AEILGKISLKIWNVTRRDSALYRCEVVARNDRKEIDEIVIELTVQVKPVTPVCRVPKAVP
VGKMATLHCQESEGHPRPHYSWYRNDVPLPTDSRANPRFRNSSFHLNSETGTLVFTAVHK
DDSGQYYCIASNDAGSARCEEQEMEVYDLN
Human VSIG1 - ECD without predicted signal sequence
(SEQ ID NO: 41)
QVSVVQVTIPDGFVNVTVGSNVTLICIYTTTVASREQLSIQWSFFHKKEMEPISHSSCLS
TEGMEEKAVSQCLKMTHARDARGRCSWTSEIYFSQGGQAVAIGQFKDRITGSNDPGNASI
TISHMQPADSGIYICDVNNPPDFLGQNQGILNVSVLVKPSKPLCSVQGRPETGHTISLSC
LSALGTPSPVYYWHKLEGRDIVPVKENFNPTTGIL
Human VSIG2 - ECD without predicted signal sequence
(SEQ ID NO: 42)
VEVKVPTEPLSTPLGKTAELTCTYSTSVGDSFALEWSFVQPGKPISESHPILYFTNGHLY
PTGSKSKRVSLLQNPPTVGVATLKLTDVHPSDTGTYLCQVNNPPDFYTNGLGLINLTVLV
PPSNPLCSQSGQTSVGGSTALRCSSSEGAPKPVYNWVRLGTFPTPSPGSMVQDEVSGQLI
LTNLSLTSSGTYRCVATNQMGSASCELTLSVTEPSQGRVA
Human VSIG3 - ECD without predicted signal sequence
(SEQ ID NO: 43)
ASLEVSESPGSIQVARGQPAVLPCTFTTSAALINLNVIWMVTPLSNANQPEQVILYQGGQ
MFDGAPRFHGRVGFTGTMPATNVSIFINNTQLSDTGTYQCLVNNLPDIGGRNIGVTGLTV
LVPPSAPHCQIQGSQDIGSDVILLCSSEEGIPRPTYLWEKLDNTLKLPPTATQDQVQGTV
TIRNISALSSGLYQCVASNAIGTSTCLLDLQVISPQPRNIGL
Human VSIG4 - ECD without predicted signal sequence
(SEQ ID NO: 44)
RPILEVPESVTGPWKGDVNLPCTYDPLQGYTQVLVKWLVQRGSDPVTIFLRDSSGDHIQQ
AKYQGRLHVSHKVPGDVSLQLSTLEMDDRSHYTCEVTWQTPDGNQVVRDKITELRVQKLS
VSKPTVTTGSGYGFTVPQGMRISLQCQARGSPPISYIWYKQQTNNQEPIKVATLSTLLFK
PAVIADSGSYFCTAKGQVGSEQHSDIVKFVVKDSSKLLKTKTEAPTTMTYPLKATSTVKQ
SWDWTTDMDGYLGETSAGPGKSLP
Human VSIG8 - ECD without predicted signal sequence
(SEQ ID NO: 45)
VRINGDGQEVLYLAEGDNVRLGCPYVLDPEDYGPNGLDIEWMQVNSDPAHHRENVFLSYQ
DKRINHGSLPHLQQRVRFAASDPSQYDASINLMNLQVSDTATYECRVKKTTMATRKVIVT
VQARPAVPMCWTEGHMTYGNDVVLKCYASGGSQPLSYKWAKISGHHYPYRAGSYTSQHSY
HSELSYQESFHSSINQGLNNGDLVLKDISRADDGLYQCTVANNVGYSVCVVEVKVSDSRR
IG
CADM Family
Human CADM1 - ECD without predicted signal sequence
(SEQ ID NO: 46)
QNLFTKDVTVIEGEVATISCQVNKSDDSVIQLLNPNRQTIYFRDFRPLKDSRFQLLNFSS
SELKVSLTNVSISDEGRYFCQLYTDPPQESYTTITVLVPPRNLMIDIQKDTAVEGEEIEV
NCTAMASKPATTIRWFKGNTELKGKSEVEEWSDMYTVTSQLMLKVHKEDDGVPVICQVEH
PAVTGNLQTQRYLEVQYKPQVHIQMTYPLQGLTREGDALELTCEAIGKPQPVMVTWVRVD
DEMPQHAVLSGPNLFINNLNKTDNGTYRCEASNIVGKAHSDYMLYVYDPPTTIPPPTTTT
TTTTTTTTTILTIITDSRAGEEGSIRAVDH
Human CADM2 - ECD without predicted signal sequence
(SEQ ID NO: 47)
QFPLTQNVTVVEGGTAILTCRVDQNDNTSLQWSNPAQQTLYFDDKKALRDNRIELVRASW
HELSISVSDVSLSDEGQYTCSLFTMPVKTSKAYLTVLGVPEKPQISGFSSPVMEGDLMQL
TCKTSGSKPAADIRWFKNDKEIKDVKYLKEEDANRKTFTVSSTLDFRVDRSDDGVAVICR
VDHESLNATPQVAMQVLEIHYTPSVKIIPSTPFPQEGQPLILTCESKGKPLPEPVLWTKD
GGELPDPDRMVVSGRELNILFLNKTDNGTYRCEATNTIGQSSAEYVLIVHDVPNTLLPTT
IIPSLTTATVTTTVAITTSPTTSATTSSIRDPNALAGQNGPDH
Human CADM3 - ECD without predicted signal sequence
(SEQ ID NO: 48)
NLSQDGYWQEQDLELGTLAPLDEALSSTVWSSPDMLASQDSQPWTSDETVVAGGTVVLKC
QVKDHEDSSLQWSNPAQQTLYFGEKRALRDNRIQLVTSTPHELSISISNVALADEGEYTC
SIFTMPVRTAKSLVTVLGIPQKPIITGYKSSLREKDTATLNCQSSGSKPAARLTWRKGDQ
ELHGEPCRIQEDPNGKTFTVSSSVTFQVTREDDGASIVCSVNHESLKGADRSTSQRIEVL
YTPTAMIRPDPPHPREGQKLLLHCEGRGNPVPQQYLWEKEGSVPPLKMTQESALIFPFLN
KSDSGT
Human CADM4 - ECD without predicted signal sequence
(SEQ ID NO: 49)
QEVQTENVTVAEGGVAEITCRLHQYDGSIVVIQNPARQTLFFNGTRALKDERFQLEEFSP
RRVRIRLSDARLEDEGGYFCQLYTEDTHHQIATLTVLVAPENPVVEVREQAVEGGEVELS
CLVPRSRPAATLRWYRDRKELKGVSSSQENGKVWSVASTVRFRVDRKDDGGIIICEAQNQ
ALPSGHSKQTQYVLDVQYSPTARIHASQAVVREGDTLVLTCAVTGNPRPNQIRWNRGNES
LPERAEAVGETLTLPGLVSADNGTYTCEASNKHGHARALYVLVVYDPGAVVEAQTSVPYA
Human CRTAM - ECD without predicted signal sequence
(SEQ ID NO: 50)
SLTNHTETITVEEGQTLTLKCVTSLRKNSSLQWLTPSGFTIFLNEYPALKNSKYQLLHHS
ANQLSITVPNVTLQDEGVYKCLHYSDSVSTKEVKVIVLATPFKPILEASVIRKQNGEEHV
VLMCSTMRSKPPPQITWLLGNSMEVSGGTLHEFETDGKKCNTTSTLIIHTYGKNSTVDCI
IRHRGLQGRKLVAPFRFEDLVTDEETASDALERNSLSSQDPQQPTSTVSVTEDSSTSEID
KEEKEQTTQDPDLTTEANPQYLGLARKKSG
Human TMIGD1 - ECD without predicted signal sequence
(SEQ ID NO: 51)
VLTVNGKTENYILDTTPGSQASLICAVQNHTREEELLWYREEGRVDLKSGNKINSSSVCV
SSISENDNGISFTCRLGRDQSVSVSVVLNVTFPPLLSGNDFQTVEEGSNVKLVCNVKANP
QAQMMWYKNSSLLDLEKSRHQIQQTSESFQLSITKVEKPDNGTYSCIAKSSLKTESLDFH
LIVKDKTVGVP
PVRFamily
Human CD96 - ECD without predicted signal sequence
(SEQ ID NO: 52)
KTVNTEENVYATLGSDVNLTCQTQTVGFFVQMQWSKVTNKIDLIAVYHPQYGFYCAYGRP
CESLVTFTETPENGSKWTLHLRNMSCSVSGRYECMLVLYPEGIQTKIYNLLIQTHVTADE
WNSNHTIEIEINQTLEIPCFQNSSSKISSEFTYAWSVENSSTDSWVLLSKGIKEDNGTQE
TLISQNHLISNSTLLKDRVKLGTDYRLHLSPVQIFDDGRKFSCHIRVGPNKILRSSTTVK
VFAKPEIPVIVENNSTDVLVERRFTCLLKNVFPKANITWFIDGSFLHDEKEGLYITNEER
KGKDGFLELKSVLTRVHSNKPAQSDNLTIWCMALSPVPGNKVWNISSEKITFLLGSEISS
TDPPLSVTESTLDTQPSPASSVSPARYPATSSVTLVDVSALRPNTTPQPSNSSMTTRGFN
YPWTSSGTDTKKSVSRIPSETYSSSPSGAGSTLHDNVFTSTARAFSEVPTTANGSTKTNH
VHITGIVVNKPKDGMS
Human CD200 - ECD without predicted signal sequence
(SEQ ID NO: 53)
QVQVVTQDEREQLYTPASLKCSLQNAQEALIVTWQKKKAVSPENMVTFSENHGVVIQPAY
KDKINITQLGLQNSTITFWNITLEDEGCYMCLFNTFGFGKISGTACLTVYVQPIVSLHYK
FSEDHLNITCSATARPAPMVFWKVPRSGIENSTVTLSHPNGTTSVTSILHIKDPKNQVGK
EVICQVLHLGTVTDFKQTVNKG
Human CD200R1 - ECD without predicted signal sequence
(SEQ ID NO: 54)
EGAAQPNNSLMLQTSKENHALASSSLCMDEKQITQNYSKVLAEVNTSWPVKMATNAVLCC
PPIALRNLIIITWEIILRGQPSCTKAYKKETNETKETNCTDERITWVSRPDQNSDLQIRT
VAITHDGYYRCIMVTPDGNFHRGYHLQVLVTPEVTLFQNRNRTAVCKAVAGKPAAHISWI
PEGDCATKQEYWSNGTVTVKSTCHWEVHNVSTVTCHVSH
Human CD200R1L- ECD without predicted signal sequence
(SEQ ID NO: 55)
SCMGGKQMTQNYSTIFAEGNISQPVLMDINAVLCCPPIALRNLIIITWEIILRGQPSCTK
AYKKETNETKETNCTVERITWVSRPDQNSDLQIRPVDTTHDGYYRGIVVTPDGNFHRGYH
LQVLVTPEVNLFQSRNITAVCKAVTGKPAAQISWLPEGSILATKQEYWGNGTVTVKSTCP
WEGHKSTVTCHVSHLTGNKSLSVKLNSGLRTSGSPALSLL
Human CD226 - ECD without predicted signal sequence
(SEQ ID NO: 56)
EEVLWHTSVPFAENMSLECVYPSMGILTQVEWFKIGTQQDSIAIFSPTHGMVIRKPYAER
VYFLNSTMASNNMTLFFRNASEDDVGYYSCSLYTYPQGTWQKVIQVVQSDSFEAAVPSNS
HIVSEPGKNVTLTCQPQMTWPVQAVRWEKIQPRQIDLLTYCNLVHGRNFTSKFPRQIVSN
CSHGRWSVIVIPDVTVSDSGLYRCYLQASAGENETFVMRLTVAEGKTDNQYTLFVA
Human PVRIG - ECD without predicted signal sequence
(SEQ ID NO: 57)
TPEVWVQVRMEATELSSFTIRCGFLGSGSISLVTVSWGGPNGAGGTTLAVLHPERGIRQW
APARQARWETQSSISLILEGSGASSPCANTTFCCKFASFPEGSWEACGSLPPSSDPGLSA
PPTPAPILRADL
Human PVR - ECD without predicted signal sequence
(SEQ ID NO: 58)
DVVVQAPTQVPGFLGDSVTLPCYLQVPNMEVTHVSQLTWARHGESGSMAVFHQTQGPSYS
ESKRLEFVAARLGAELRNASLRMFGLRVEDEGNYTCLFVTFPQGSRSVDIWLRVLAKPQN
TAEVQKVQLTGEPVPMARCVSTGGRPPAQITWHSDLGGMPNTSQVPGFLSGTVTVTSLWI
LVPSSQVDGKNVTCKVEHESFEKPQLLTVNLTVYYPPEVSISGYDNNWYLGQNEATLTCD
ARSNPEPTGYNWSTTMGPLPPFAVAQGAQLLIRPVDKPINTTLICNVTNALGARQAELTV
QVKEGPPSEHSGMSRN
Human PVRL1 - ECD without predicted signal sequence
(SEQ ID NO: 59)
QVVQVNDSMYGFIGTDVVLHCSFANPLPSVKITQVTWQKSTNGSKQNVAIYNPSMGVSVL
APYRERVEFLRPSFTDGTIRLSRLELEDEGVYICEFATFPTGNRESQLNLTVMAKPTNWI
EGTQAVLRAKKGQDDKVLVATCTSANGKPPSVVSWETRLKGEAEYQEIRNPNGTVTVISR
YRLVPSREAHQQSLACIVNYHMDRFKESLTLNVQYEPEVTIEGFDGNWYLQRMDVKLTCK
ADANPPATEYHWTTLNGSLPKGVEAQNRTLFFKGPINYSLAGTYTCEATNPIGTRSGQVE
VNITEFPYTPSPPEHGRRAGPVPTA
Human PVRL2 - ECD without predicted signal sequence
(SEQ ID NO: 60)
QDVRVQVLPEVRGQLGGTVELPCHLLPPVPGLYISLVTWQRPDAPANHQNVAAFHPKMGP
SFPSPKPGSERLSFVSAKQSTGQDTEAELQDATLALHGLTVEDEGNYTCEFATFPKGSVR
GMTWLRVIAKPKNQAEAQKVTFSQDPTTVALCISKEGRPPARISWLSSLDWEAKETQVSG
TLAGTVTVTSRFTLVPSGRADGVTVTCKVEHESFEEPALIPVTLSVRYPPEVSISGYDDN
WYLGRTDATLSCDVRSNPEPTGYDWSTTSGTFPTSAVAQGSQLVIHAVDSLFNTTFVCTV
TNAVGMGRAEQVIFVRETPNTAGAGATGG
Human PVRL3 - ECD without predicted signal sequence
(SEQ ID NO: 61)
GPIIVEPHVTAVWGKNVSLKCLIEVNETITQISWEKIHGKSSQTVAVHHPQYGFSVQGEY
QGRVLFKNYSLNDATITLHNIGFSDSGKYICKAVTFPLGNAQSSTTVTVLVEPTVSLIKG
PDSLIDGGNETVAAICIAATGKPVAHIDWEGDLGEMESTTTSFPNETATIISQYKLFPTR
FARGRRITCVVKHPALEKDIRYSFILDIQYAPEVSVTGYDGNWFVGRKGVNLKCNADANP
PPFKSVWSRLDGQWPDGLLASDNTLHFVHPLTFNYSGVYICKVTNSLGQRSDQKVIYISD
PPTTTTLQPTIQWHPSTADIEDLATEPKKLPFPLSTLATIKDDTIAT
Human PVRL4 - ECD without predicted signal sequence
(SEQ ID NO: 62)
GELETSDVVTVVLGQDAKLPCFYRGDSGEQVGQVAWARVDAGEGAQELALLHSKYGLHVS
PAYEGRVEQPPPPRNPLDGSVLLRNAVQADEGEYECRVSTFPAGSFQARLRLRVLVPPLP
SLNPGPALEEGQGLTLAASCTAEGSPAPSVTWDTEVKGTTSSRSFKHSRSAAVTSEFHLV
PSRSMNGQPLTCVVSHPGLLQDQRITHILHVSFLAEASVRGLEDQNLWHIGREGAMLKCL
SEGQPPPSYNWTRLDGPLPSGVRVDGDTEGFPPLTTEHSGIYVCHVSNEFSSRDSQVTVD
VLDPQEDSGKQVDLVSAS
Human TIGIT - ECD without predicted signal sequence
(SEQ ID NO: 63)
MMTGTIETTGNISAEKGGSIILQCHLSSTTAQVTQVNWEQQDQLLAICNADLGWHISPSF
KDRVAPGPGLGLTLQSLTVNDTGEYFCIYHTYPDGTYTGRIFLEVLESSVAEHGARFQIP
LLGAMAATLVVICTAVIVVVA
TMEM25 - ECD without predicted signal sequence
(SEQ ID NO: 64)
ELEPQIDGQTWAERALRENERHAFTCRVAGGPGTPRLAWYLDGQLQEASTSRLLSVGGEA
FSGGTSTFTVTAHRAQHELNCSLQDPRSGRSANASVILNVQFKPEIAQVGAKYQEAQGPG
LLVVLFALVRANPPANVTWIDQDGPVTVNTSDFLVLDAQNYPWLTNHTVQLQLRSLAHNL
SVVATNDVGVTSASLPAPGLLATRVE
Human IgG₁ Fc region
(SEQ ID NO: 65)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region
(SEQ ID NO: 66)
KSSDKTHTCPPCPAPELLGGPSVFLEPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human IgG₁ Fc region
(SEQ ID NO: 67)
EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWENGKEYKCKVSNKALPAPIEKT
ISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
dnYAP
(SEQ ID NO: 68)
MDPGQQPPPQPAPQGQGQPPSQPPQGQGPPSGPGQPAPAATQAAPQAPPAGHQIVHVRGD
SETDLEALFNAVMNPKTANVPQTVPMRLRKLPDSFFKPPEPKSHSRQASTDAGTAGALTP
QHVRAHASPASLQLGAVSPGTLTPTGVVSGPAATPTAQHLRQSSSEIPDDVPLPAGWEMA
KTSSGQRYFLNHIDQTTTWQDPRKAMLSQMNVTAPTSPPVQQNMMNSASGPLPDGWEQAM
TQDGEIYYINHKNKITSWLDPRLDPRFAMNQRISQSAPVKQPPPLAPQSPQGGVMGGSNL
NDMESVLAATKLDKESFLTWL
YFP
(SEQ ID NO: 69)
MVSKGEELFTGVVPILVELDGDVNGHKFSVSGEGEGDATYGKLTLKFICTTGKLPVPWPT
LVTTFGYGLQCFARYPDHMKQHDFFKSAMPEGYVQERTIFFKDDGNYKTRAEVKFEGDTL
VNRIELKGIDFKEDGNILGHKLEYNYNSHNVYIMADKQKNGIKVNFKIRHNIEDGSVQLA
DHYQQNTPIGDGPVLLPDNHYLSYQSALSKDPNEKRDHMVLLEFVTAAGITLGMDELYK
Human TEAD2
(SEQ ID NO: 70)
MGEPRAGAALDDGSGWTGSEEGSEEGTGGSEGAGGDGGPDAEGVWSPDIEQSFQEALAIY
PPCGRRKIILSDEGKMYGRNELIARYIKLRTGKTRTRKQVSSHIQVLARRKSREIQSKLK
DQVSKDKAFQTMATMSSAQLISAPSLQAKLGPTGPQASELFQFWSGGSGPPWNVPDVKPF
SQTPFTLSLTPPSTDLPGYEPPQALSPLPPPTPSPPAWQARGLGTARLQLVEFSAFVEPP
DAVDSYQRHLFVHISQHCPSPGAPPLESVDVRQIYDKFPEKKGGLRELYDRGPPHAFFLV
KFWADLNWGPSGEEAGAGGSISSGGFYGVSSQYESLEHMTLTCSSKVCSFGKQVVEKVET
ERAQLEDGRFVYRLLRSPMCEYLVNFLHKLRQLPERYMMNSVLENFTILQVVTNRDTQEL
LLCTAYVFEVSTSERGAQHHIYRLVRD
Human TEAD3
(SEQ ID NO: 71)
MASNSWNASSSPGEAREDGPEGLDKGLDNDAEGVWSPDIEQSFQEALAIYPPCGRRKIIL
SDEGKMYGRNELIARYIKLRTGKTRTRKQVSSHIQVLARKKVREYQVGIKAMNLDQVSKD
KALQSMASMSSAQIVSASVLQNKFSPPSPLPQAVFSTSSRFWSSPPLLGQQPGPSQDIKP
FAQPAYPIQPPLPPTLSSYEPLAPLPSAAASVPVWQDRTIASSRLRLLEYSAFMEVQRDP
DTYSKHLFVHIGQTNPAFSDPPLEAVDVRQIYDKEPEKKGGLKELYEKGPPNAFFLVKFW
ADLNSTIQEGPGAFYGVSSQYSSADSMTISVSTKVCSFGKQVVEKVETEYARLENGRFVY
RIHRSPMCEYMINFIHKLKHLPEKYMMNSVLENFTILQVVTSRDSQETLLVLAFVFEVST
SEHGAQHHVYKLVKD
Linker
(SEQ ID NO: 72)
RPACKIPNDLKQKVMNH
FLAG Tag
(SEQ ID NO: 73)
DYKDDDDK
Linker
(SEQ ID NO: 74)
ESGGGGVT
Linker
(SEQ ID NO: 75)
LESGGGGVT
Linker
(SEQ ID NO: 76)
GRAQVT
Linker
(SEQ ID NO: 77)
WRAQVT
Linker
(SEQ ID NO: 78)
ARGRAQVT
Human IgG2 Heavy chain constant region
(SEQ ID NO: 79)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGN
VFSCSVMHEALHNHYTQKSLSLSPGK

1-80. (canceled)

81. An agent that specifically binds the extracellular domain of one or more cell adhesion molecules of the immunoglobulin superfamily (IgCAM), wherein the IgCAM is selected from the group consisting of: AMICA, CAR, CLMP, ESAM, GPA33, VSIG1, VSIG2, VSIG3, VSIG4, VSIG8, JAM1, JAM2, JAM3, CADM1, CADM2, CADM3, CADM4, CRTAM, TMIGD1, PVR, PVRL1, PVRL2, PVRL3, PVRL4, PVRIG, CD200, CD200R1, CD200R1L, CD226, CD96, TIGIT, and TMEM25.

82. The agent of claim 81, which is a soluble receptor.

83. The agent of claim 82, wherein the soluble receptor comprises:
(a) the extracellular domain of an IgCAM or a portion thereof;
(b) a sequence selected from the group consisting of: SEQ ID NOs:33-64; or
(c) a portion of a sequence selected from the group consisting of: SEQ ID NOs:33-64.

84. The agent of claim 82, wherein the soluble receptor comprises the extracellular domain of CADM1, CADM3, PVRL1, PVRL2, or PVRL3, or a portion thereof.

85. The agent of claim 82, which further comprises a Fc region.

86. The agent of claim 81, which is an antibody.

87. The agent of claim 86, wherein the antibody is a monoclonal antibody, a recombinant antibody, a chimeric antibody, a humanized antibody, a human antibody, an antibody fragment comprising an antigen-binding site, or a bispecific antibody.

88. The agent of claim 81, which:
(a) modulates the Hippo pathway;
(b) activates or increases Hippo pathway signaling;
(c) reduces YAP activity;
(d) increases phosphorylated YAP;
(e) decreases the expression of YAP-dependent genes; and/or
(f) decreases the expression of at least one of the genes selected from the group consisting of: CD44, CD47, CD133, TDGF1, EPHB2, CA12, LRP4, GPC4, CLDN2, CTGF, PAG1, SEMA4D, RHEB, MAGI1, ITPR3, CD168, NRP2, Gli2, Birc2, Birc5, FGF1, IL33, GRB2, IGFBP3, and AREG.

89. The agent of claim 81, which:
(a) modulates apoptosis;
(b) modulates cellular proliferation; and/or
(c) modulates epithelial mesenchymal transition (EMT).

90. The agent of claim 81, which inhibits the growth of a tumor or tumor cells.

91. The agent of claim 90, wherein the tumor is a tumor selected from the group consisting of colorectal tumor, colon tumor, pancreatic tumor, lung tumor, ovarian tumor, liver tumor, breast tumor, kidney tumor, prostate tumor, melanoma, cervical tumor, bladder tumor, brain tumor, gastrointestinal tumor, and head and neck tumor.

92. A pharmaceutical composition comprising the agent of claim 81 and a pharmaceutically acceptable carrier.

93. A cell producing the agent of claim 81.

94. A polynucleotide comprising a polynucleotide that encodes the agent of claim 81.

95. A method of inhibiting tumor growth comprising contacting a tumor or tumor cell with the agent of claim 81.

96. The method of claim 95, wherein the tumor is a tumor selected from the group consisting of colorectal tumor, colon tumor, pancreatic tumor, lung tumor, ovarian tumor, liver tumor, breast tumor, kidney tumor, prostate tumor, melanoma, cervical tumor, bladder tumor, brain tumor, gastrointestinal tumor, and head and neck tumor.

97. A method of treating cancer in a subject, comprising administering a therapeutically effective amount of the agent of claim 81 to the subject.

98. The method of claim 97, wherein the cancer is selected from the group consisting of: colorectal cancer, colon cancer, pancreatic cancer, lung cancer, ovarian cancer, liver cancer, breast cancer, kidney cancer, prostate cancer, melanoma, cervical cancer, bladder cancer, brain cancer, gastrointestinal cancer, and head and neck cancer.

99. The method of claim 97, which further comprises administering a therapeutically effective amount of a second therapeutic agent to the subject.

100. The method of claim 99, wherein the second therapeutic agent is a chemotherapeutic agent or an antibody.

101. A method of modulating the Hippo pathway in a cell, comprising contacting the cell with the agent of claim 81.
